SEQUENCE LISTING (200 sequences)

SEQ ID NO: 1 - RNA, 23 nt, Homo sapiens
  source 1..23: mol_type = genomic RNA; note = position 378-400 target sequence
tctaaagattcctgctttacaga

SEQ ID NO: 2 - RNA, 21 nt, synthetic construct
  source 1..21: mol_type = other RNA; note = position 378-400 RNA guide
tgtaaagcaggaatctttaga

SEQ ID NO: 3 - RNA, 23 nt, Homo sapiens
  source 1..23: mol_type = genomic RNA; note = position 387-409 target sequence
tcctgctttacagaaaacactcc

SEQ ID NO: 4 - RNA, 21 nt, Homo sapiens
  source 1..21: mol_type = other RNA; note = position 387-409 RNA guide
agtgttttctgtaaagcagga

SEQ ID NO: 5 - RNA, 23 nt, Homo sapiens
  source 1..23: mol_type = genomic RNA; note = position 1296-1318 target sequence
atgatcttagtagccatattttc

SEQ ID NO: 6 - RNA, 21 nt, synthetic construct
  source 1..21: mol_type = other RNA; note = position 1296-1318 RNA guide
aaatatggctactaagatcat

SEQ ID NO: 7 - RNA, 23 nt, Homo sapiens
  source 1..23: mol_type = genomic RNA; note = position 1299-1321 target sequence
atcttagtagccatattttcaga

SEQ ID NO: 8 - RNA, 21 nt, synthetic construct
  source 1..21: mol_type = other RNA; note = position 1299-1321 RNA guide
tgaaaatatggctactaagat

SEQ ID NO: 9 - RNA, 23 nt, Homo sapiens
  source 1..23: mol_type = genomic RNA; note = position 1332-1354 target sequence
ttcgattattgtgctgtaaaggc

SEQ ID NO: 10 - RNA, 21 nt, synthetic construct
  source 1..21: mol_type = other RNA; note = position 1332-1354 RNA guide
ctttacagcacaataatcgaa

SEQ ID NO: 11 - RNA, 23 nt, Homo sapiens
  source 1..23: mol_type = genomic RNA; note = position 1522-1544 target sequence
cagaagtgatcttcacaaacatt

SEQ ID NO: 12 - RNA, 21 nt, synthetic construct
  source 1..21: mol_type = other RNA; note = position 1522-1544 RNA guide
tgtttgtgaagatcacttctg

SEQ ID NO: 13 - RNA, 23 nt, Homo sapiens
  source 1..23: mol_type = genomic RNA; note = position 1571-1593 target sequence
ccctccaaatgtcattattgttt

SEQ ID NO: 14 - RNA, 21 nt, synthetic construct
  source 1..21: mol_type = other RNA; note = position 1571-1593 RNA guide
acaataatgacatttggaggg

SEQ ID NO: 15 - RNA, 23 nt, Homo sapiens
  source 1..23: mol_type = genomic RNA; note = position 1697-1719 target sequence
actcctttactggaacagaaagc

SEQ ID NO: 16 - RNA, 21 nt, synthetic construct
  source 1..21: mol_type = other RNA; note = position 1697-1719 RNA guide
tttctgttccagtaaaggagt

SEQ ID NO: 17 - RNA, 23 nt, Homo sapiens
  source 1..23: mol_type = genomic RNA; note = position 2002-2024 target sequence
tgggatttgttatcttcatgttt

SEQ ID NO: 18 - RNA, 21 nt, synthetic construct
  source 1..21: mol_type = other RNA; note = position 2002-2024 RNA guide
acatgaagataacaaatccca

SEQ ID NO: 19 - RNA, 23 nt, Homo sapiens
  source 1..23: mol_type = genomic RNA; note = position 2215-2237 target sequence
tagaatatgttggagaacaaact

SEQ ID NO: 20 - RNA, 21 nt, synthetic construct
  source 1..21: mol_type = other RNA; note = position 2215-2237 RNA guide
tttgttctccaacatattcta

SEQ ID NO: 21 - RNA, 23 nt, Homo sapiens
  source 1..23: mol_type = genomic RNA; note = position 2227-2249 target sequence
gagaacaaactgctttgctaata

SEQ ID NO: 22 - RNA, 21 nt, synthetic construct
  source 1..21: mol_type = other RNA; note = position 2227-2249 RNA guide
ttagcaaagcagtttgttctc

SEQ ID NO: 23 - RNA, 23 nt, Homo sapiens
  source 1..23: mol_type = genomic RNA; note = position 2228-2250 target sequence
agaacaaactgctttgctaataa

SEQ ID NO: 24 - RNA, 21 nt, synthetic construct
  source 1..21: mol_type = other RNA; note = position 2228-2250 RNA guide
attagctttgcagtttgttct

SEQ ID NO: 25 - RNA, 23 nt, Homo sapiens
  source 1..23: mol_type = genomic RNA; note = position 2229-2251 target sequence
gaacaaactgctttgctaataaa

SEQ ID NO: 26 - RNA, 21 nt, synthetic construct
  source 1..21: mol_type = other RNA; note = position 2229-2251 RNA guide
tattagcaaagcagtttgttc

SEQ ID NO: 27 - RNA, 23 nt, Homo sapiens
  source 1..23: mol_type = genomic RNA; note = DNA position 2639-2661 target sequence
catctattgctcgaagactaaag

SEQ ID NO: 28 - RNA, 21 nt, synthetic construct
  source 1..21: mol_type = other RNA; note = position 2639-2661 RNA guide
ttagtcttcgagcaatagatg

SEQ ID NO: 29 - RNA, 23 nt, Homo sapiens
  source 1..23: mol_type = genomic RNA; note = position 2640-2662 target sequence
atctattgctcgaagactaaagg

SEQ ID NO: 30 - RNA, 21 nt, synthetic construct
  source 1..21: mol_type = other RNA; note = position 2640-2662 RNA guide
tttagtcttcgagcaatagat

SEQ ID NO: 31 - RNA, 23 nt, Homo sapiens
  source 1..23: mol_type = genomic RNA; note = position 2652-2674 target sequence
aagactaaaggaaacttcatcca

SEQ ID NO: 32 - RNA, 21 nt, synthetic construct
  source 1..21: mol_type = other RNA; note = position 2652-2674 RNA guide
gatgaagtttcctttagtctt

SEQ ID NO: 33 - RNA, 23 nt, Homo sapiens
  source 1..23: mol_type = genomic RNA; note = position 2671-2693 target sequence
tccatcacaacccattagtcata

SEQ ID NO: 34 - RNA, 21 nt, synthetic construct
  source 1..21: mol_type = other RNA; note = position 2671-2693 RNA guide
tgactaatgggttgtgatgga

SEQ ID NO: 35 - RNA, 23 nt, Homo sapiens
  source 1..23: mol_type = genomic RNA; note = position 2679-2701 target sequence
aacccattagtcataaaactacc

SEQ ID NO: 36 - RNA, 21 nt, synthetic construct
  source 1..21: mol_type = other RNA; note = position 2679-2701 RNA guide
tagttttatgactaatgggtt

SEQ ID NO: 37 - RNA, 23 nt, Homo sapiens
  source 1..23: mol_type = genomic RNA; note = position 2680-2702 target sequence
acccattagtcataaaactaccc

SEQ ID NO: 38 - RNA, 21 nt, synthetic construct
  source 1..21: mol_type = other RNA; note = position 2680-2702 RNA guide
gtagttttatgactaatgggt

SEQ ID NO: 39 - RNA, 23 nt, Homo sapiens
  source 1..23: mol_type = genomic RNA; note = position 2707-2729 target sequence
cccactgtttgaagaagaaaagg

SEQ ID NO: 40 - RNA, 21 nt, synthetic construct
  source 1..21: mol_type = other RNA; note = position 2707-2729 RNA guide
ttttcttcttcaaacagtggg

SEQ ID NO: 41 - RNA, 23 nt, Homo sapiens
  source 1..23: mol_type = genomic RNA; note = position 2773-2795 target sequence
tgacactaacactctaattcaag

SEQ ID NO: 42 - RNA, 21 nt, synthetic construct
  source 1..21: mol_type = other RNA; note = position 2773-2795 RNA guide
tgaattagagtgttagtgtca

SEQ ID NO: 43 - RNA, 23 nt, Homo sapiens
  source 1..23: mol_type = genomic RNA; note = position 2774-2796 target sequence
gacactaacactctaattcaagc

SEQ ID NO: 44 - RNA, 21 nt, synthetic construct
  source 1..21: mol_type = other RNA; note = position 2774-2796 RNA guide
ttgaattagagtgttagtgtc

SEQ ID NO: 45 - RNA, 23 nt, Homo sapiens
  source 1..23: mol_type = genomic RNA; note = position 2841-2863 target sequence
aaggacaaaatgtagaaagatgt

SEQ ID NO: 46 - RNA, 21 nt, synthetic construct
  source 1..21: mol_type = other RNA; note = position 2841-2863 RNA guide
atctttctacattttgtcctt

SEQ ID NO: 47 - RNA, 23 nt, Homo sapiens
  source 1..23: mol_type = genomic RNA; note = position 2850-2872 target sequence
atgtagaaagatgtgagataact

SEQ ID NO: 48 - RNA, 21 nt, synthetic construct
  source 1..21: mol_type = other RNA; note = position 2850-2872 RNA guide
ttatctcacatctttctacat

SEQ ID NO: 49 - RNA, 23 nt, Homo sapiens
  source 1..23: mol_type = genomic RNA; note = position 2853-2875 target sequence
tagaaagatgtgagataacttac

SEQ ID NO: 50 - RNA, 21 nt, synthetic construct
  source 1..21: mol_type = other RNA; note = position 2853-2875 RNA guide
aagttatctcacatctttcta

SEQ ID NO: 51 - RNA, 23 nt, Homo sapiens
  source 1..23: mol_type = genomic RNA; note = position 2921-2943 target sequence
tgctatacataggaaaagacaca

SEQ ID NO: 52 - RNA, 21 nt, synthetic construct
  source 1..21: mol_type = other RNA; note = position 2921-2943 RNA guide
tgtcttttcctatgtatagca

SEQ ID NO: 53 - RNA, 23 nt, Homo sapiens
  source 1..23: mol_type = genomic RNA; note = position 2964-2986 target sequence
tactgtttaactgtcaattctcc

SEQ ID NO: 54 - RNA, 21 nt, synthetic construct
  source 1..21: mol_type = other RNA; note = position 2964-2986 RNA guide
agaattgacagttaaacagta

SEQ ID NO: 55 - RNA, 23 nt, Homo sapiens
  source 1..23: mol_type = genomic RNA; note = position 3045-3067 target sequence
ttcaaattgtatgctttctgaat

SEQ ID NO: 56 - RNA, 21 nt, synthetic construct
  source 1..21: mol_type = other RNA; note = position 3045-3067 RNA guide
tcagaaagcatacaatttgaa

SEQ ID NO: 57 - RNA, 23 nt, Homo sapiens
  source 1..23: mol_type = genomic RNA; note = position 3055-3077 target sequence
atgctttctgaatatagactttc

SEQ ID NO: 58 - RNA, 21 nt, synthetic construct
  source 1..21: mol_type = other RNA; note = position 3055-3077 RNA guide
aagtctatattcagaaagcat

SEQ ID NO: 59 - RNA, 23 nt, Homo sapiens
  source 1..23: mol_type = genomic RNA; note = position 3069-3091 target sequence
tagactttctgggaaaaggttta

SEQ ID NO: 60 - RNA, 21 nt, synthetic construct
  source 1..21: mol_type = other RNA; note = position 3069-3091 RNA guide
aaccttttcccagaaagtcta

SEQ ID NO: 61 - RNA, 23 nt, Homo sapiens
  source 1..23: mol_type = genomic RNA; note = position 3092-3114 target sequence
ctgctcgtaaggaaacattttat

SEQ ID NO: 62 - RNA, 21 nt, synthetic construct
  source 1..21: mol_type = other RNA; note = position 378 RNA guide
aaaatgtttccttacgagcag

SEQ ID NO: 63 - RNA, 23 nt, Homo sapiens
  source 1..23: mol_type = genomic RNA; note = position 3093-3115 target sequence
tgctcgtaaggaaacattttatg

SEQ ID NO: 64 - RNA, 21 nt, synthetic construct
  source 1..21: mol_type = other RNA; note = position 3093-3115 RNA guide
taaaatgtttccttacgagca

SEQ ID NO: 65 - RNA, 3143 nt, Homo sapiens
  source 1..3143: mol_type = genomic RNA; note = OCA2 transcript variant 1
gttcttacttcgaaggctgtgctccgctcaccatccagagcggaggtgcggaccttaaactcactcctggagaaagatctgcaagtgcgcagagagaagactggcagtggagcatgcatctggagggcagagacggcaggcggtaccccggcgcgccggcggtggagctcctgcagacgtccgtgcccagcggactcgctgaacttgtggccggcaagcgcaggcttcctcggggagccggtggagctgacccctcgcactcctgccccaggggggctgccgggcagagctcttgggctcctgcaggccaggagtttgcttcattcctcacaaaagggaggtctcactcttctttgccccagatgtccagctccaggtctaaagattcctgctttacagaaaacactcctttgctgaggaattccttacaggagaaagggtcacggtgcatacctgtttaccatccagagttcatcactgctgaagagtcttgggaagacagctctgctgactgggagcgaagatacctgctaagcagggaggtgtctggtctgtctgcatctgcctcctccgagaagggagaccttctggacagcccgcacatccgactccgtctttccaagctgaggcgctgtgtgcagtggctgaaagtcatgggcctgtttgcctttgtggtgctgtgttctattttgttcagcctatatccggatcaaggaaagctctggcagctgttggccttatcaccgctggagaactactccgtgaaccttagcagccacgtggactccacgctgctgcaggtggacctggcaggggccctagtggccagtgggccgagtcgtcctgggagggaagagcacatcgtggtggagctgacccaggctgacgctttgggctccaggtggcggcggccacagcaggtcactcacaactggacggtgtatttaaatccgaggagaagcgagcactcagtgatgagcaggacctttgaggtactgaccagagagacggtgtccatcagcatccgggcctccctgcagcagacccaggctgtccctcttttgatggctcatcagtacctccgcggaagtgtagaaacccaggtgaccatcgcgacggccatcctcgcgggcgtctacgcgctgatcatatttgagatcgtgcacagaactctggcggccatgctgggttcccttgcagcactggcagcactggctgtgattggcgatagacccagcctgacccatgtggtggagtggattgattttgagacgctggccctgctgtttggcatgatgatcttagtagccatattttcagaaacgggatttttcgattattgtgctgtaaaggcataccggctctcccggggacgggtgtgggccatgatcatcatgctctgtctcatcgcggccgtcctctctgccttcttggacaacgtcaccaccatgctcctcttcacgcctgtgaccataaggttgtgtgaggtgctcaaccttgatccaagacaagtcctgattgcagaagtgatcttcacaaacattggaggagctgccactgccatcggggaccctccaaatgtcattattgtttccaaccaagagctgaggaagatgggcctggactttgccggattcactgcacacatgttcattgggatttgccttgttctcctggtctgctttccgctcctcagactcctttactggaacagaaagctttataacaaggaacccagtgagattgttgaactgaagcacgagattcacgtctggcgcctgactgctcagcgcatcagcccggccagccgcgaggagacagctgtgcgccgcctgctgctggggaaggtgctggcactggagcacctgctcgcccggaggctgcacaccttccacagacagatctcacaggaggacaaaaattgggagaccaatatccaagaactccaaaaaaagcataggatatctgacgggattctgctcgccaaatgcctgacagtgttgggatttgttatcttcatgtttttcctcaattcgtttgtccctggcattcatcttgatcttggatggattgctattctgggtgccatctggttgctaattttagctgatattcatgattttgagataattctacacagagtggaatgggcaacccttctgttttttgcagcgctctttgttctgatggaggcattggcacatctccacttaatagaatatgttggagaacaaactgctttgctaataaagatggtcccagaggagcagcgcctcatagccgccattgtcctggtggtgtgggtctcagccctggcgtcgtccctgattgacaacatcccgttcactgctaccatgattcccgtgctcctgaacctgagccacgaccctgaggttggcctgcccgcaccgccgctcatgtatgccctggccttcggtgcttgcctgggaggtaacgggacactgattggcgcgtcggcaaacgtcgtgtgtgcagggattgcagaacagcatggatatgggttctccttcatggaatttttcaggctgggcttcccaatgatggttgtgtcctgcactgttgggatgtgttatctccttgtggctcatgtggtggtgggatggaattaatagacatccatctattgctcgaagactaaaggaaacttcatccatcacaacccattagtcataaaactaccctgaccccactgtttgaagaagaaaaggtgcttaccctggagatgctacagagacacagtggaatagaccttgacactaacactctaattcaagcgaatgttggaacaccatgacctcctctgtgtgtcctttctccccaaggacaaaatgtagaaagatgtgagataacttactcaagattcccctccagaaaaatacgtatgtttaaaaacccttcctgctatacataggaaaagacacacatccacctaaaattgactgtactgtttaactgtcaattctcctgaggctaaacacagtttgtttttcttgtaatcacttttcatgttaaaataatcagcattcaaattgtatgctttctgaatatagactttctgggaaaaggtttactgctcgtaaggaaacattttatgtattaaaataaactgttccttgataaaa

SEQ ID NO: 66 - protein, 838 aa, Homo sapiens
  source 1..838: note = OCA2 protein
MHLEGRDGRRYPGAPAVELLQTSVPSGLAELVAGKRRLPRGAGGADPSHSCPRGAAGQSSWAPAGQEFASFLTKGRSHSSLPQMSSSRSKDSCFTENTPLLRNSLQEKGSRCIPVYHPEFITAEESWEDSSADWERRYLLSREVSGLSASASSEKGDLLDSPHIRLRLSKLRRCVQWLKVMGLFAFVVLCSILFSLYPDQGKLWQLLALSPLENYSVNLSSHVDSTLLQVDLAGALVASGPSRPGREEHIVVELTQADALGSRWRRPQQVTHNWTVYLNPRRSEHSVMSRTFEVLTRETVSISIRASLQQTQAVPLLMAHQYLRGSVETQVTIATAILAGVYALIIFEIVHRTLAAMLGSLAALAALAVIGDRPSLTHVVEWIDFETLALLFGMMILVAIFSETGFFDYCAVKAYRLSRGRVWAMIIMLCLIAAVLSAFLDNVTTMLLFTPVTIRLCEVLNLDPRQVLIAEVIFTNIGGAATAIGDPPNVIIVSNQELRKMGLDFAGFTAHMFIGICLVLLVCFPLLRLLYWNRKLYNKEPSEIVELKHEIHVWRLTAQRISPASREETAVRRLLLGKVLALEHLLARRLHTFHRQISQEDKNWETNIQELQKKHRISDGILLAKCLTVLGFVIFMFFLNSFVPGIHLDLGWIAILGAIWLLILADIHDFEIILHRVEWATLLFFAALFVLMEALAHLHLIEYVGEQTALLIKMVPEEQRLIAAIVLVVWVSALASSLIDNIPFTATMIPVLLNLSHDPEVGLPAPPLMYALAFGACLGGNGTLIGASANVVCAGIAEQHGYGFSFMEFFRLGFPMMVVSCTVGMCYLLVAHVVVGWN

SEQ ID NO: 67 - RNA, 21 nt, synthetic construct
  source 1..21: mol_type = other RNA; note = siRNA sense strand for targeting OCA2 in human
taaagattcctgctttacaga

SEQ ID NO: 68 - RNA, 21 nt, synthetic construct
  source 1..21: mol_type = other RNA; note = siRNA sense strand for targeting OCA2 in Oryctolagus cuniculus
gcatctagagaacaaagatgg

SEQ ID NO: 69 - RNA, 21 nt, synthetic construct
  source 1..21: mol_type = other RNA; note = siRNA antisense strand for targeting OCA2 in Oryctolagus cuniculus
atctttgttctctagatgcat

SEQ ID NO: 70 - RNA, 21 nt, synthetic construct
  source 1..21: mol_type = other RNA; note = OCA2 siRNA #1 sense for Mus musculus
gatcatatttgagattgtttt

SEQ ID NO: 71 - RNA, 21 nt, synthetic construct
  source 1..21: mol_type = other RNA; note = OCA2 siRNA #1 antisense for Mus musculus
aacaatctcaaatatgatcag

SEQ ID NO: 72 - RNA, 21 nt, synthetic construct
  source 1..21: mol_type = other RNA; note = OCA2 siRNA #2 sense for Mus musculus
cgatgattcctgtactccttt

SEQ ID NO: 73 - RNA, 21 nt, synthetic construct
  source 1..21: mol_type = other RNA; note = OCA2 siRNA #2 antisense for Mus musculus
aggagtacaggaatcatcgta

SEQ ID NO: 74 - RNA, 21 nt, synthetic construct
  source 1..21: mol_type = other RNA; note = OCA2 siRNA #3 sense for Mus musculus
ggttgctaattttagctgatt

SEQ ID NO: 75 - RNA, 21 nt, synthetic construct
  source 1..21: mol_type = other RNA; note = OCA2 siRNA #3 antisense for Mus musculus
tcagctaaaattagcaaccag

SEQ ID NO: 76 - RNA, 25 nt, synthetic construct
  source 1..25: mol_type = other RNA; note = siRNA sense targeting SOX10 for Mus musculus
agccagtatatacgactctatcccg

SEQ ID NO: 77 - RNA, 27 nt, synthetic construct
  source 1..27: mol_type = other RNA; note = siRNA antisense targeting SOX10 for Mus musculus
cgtcggtcatatatgctgagatagggc

SEQ ID NO: 78 - RNA, 23 nt, Homo sapiens
  source 1..23: mol_type = genomic RNA; note = target sequence position 1297-1319
tgatcttagtagccatattttca

SEQ ID NO: 79 - RNA, 23 nt, Homo sapiens
  source 1..23: mol_type = genomic RNA; note = target sequence position 1323-1345
acgggatttttcgattattgtgc

SEQ ID NO: 80 - RNA, 23 nt, Homo sapiens
  source 1..23: mol_type = genomic RNA; note = target sequence position 1916-1938
caaaaattgggagaccaatatcc

SEQ ID NO: 81 - RNA, 23 nt, Homo sapiens
  source 1..23: mol_type = genomic RNA; note = target sequence position 1944-1966
ctccaaaaaaagcataggatatc

SEQ ID NO: 82 - RNA, 23 nt, Homo sapiens
  source 1..23: mol_type = genomic RNA; note = target sequence position 1945-1967
tccaaaaaaagcataggatatct

SEQ ID NO: 83 - RNA, 23 nt, Homo sapiens
  source 1..23: mol_type = genomic RNA; note = target sequence position 2003-2025
gggatttgttatcttcatgtttt

SEQ ID NO: 84 - RNA, 23 nt, Homo sapiens
  source 1..23: mol_type = genomic RNA; note = target sequence position 2006-2028
atttgttatcttcatgtttttcc

SEQ ID NO: 85 - RNA, 23 nt, Homo sapiens
  source 1..23: mol_type = genomic RNA; note = target sequence position 2019-2041
atgtttttcctcaattcgtttgt

SEQ ID NO: 86 - RNA, 23 nt, Homo sapiens
  source 1..23: mol_type = genomic RNA; note = target sequence position 2094-2116
ttgctaattttagctgatattca

SEQ ID NO: 87 - RNA, 23 nt, Homo sapiens
  source 1..23: mol_type = genomic RNA; note = target sequence position 2096-2118
gctaattttagctgatattcatg

SEQ ID NO: 88 - RNA, 23 nt, Homo sapiens
  source 1..23: mol_type = genomic RNA; note = target sequence position 2105-2127
agctgatattcatgattttgaga

SEQ ID NO: 89 - RNA, 23 nt, Homo sapiens
  source 1..23: mol_type = genomic RNA; note = target sequence position 2111-2133
tattcatgattttgagataattc

SEQ ID NO: 90 - RNA, 23 nt, Homo sapiens
  source 1..23: mol_type = genomic RNA; note = homo sapiens target sequence position 2112-2134
attcatgattttgagataattct

SEQ ID NO: 91 - RNA, 23 nt, Homo sapiens
  source 1..23: mol_type = genomic RNA; note = target sequence position 2115-2137
catgattttgagataattctaca

SEQ ID NO: 92 - RNA, 23 nt, Homo sapiens
  source 1..23: mol_type = genomic RNA; note = target sequence position 2205-2227
ctccacttaatagaatatgttgg

SEQ ID NO: 93 - RNA, 23 nt, Homo sapiens
  source 1..23: mol_type = genomic RNA; note = target sequence position 2626-2648
attaatagacatccatctattgc

SEQ ID NO: 94 - RNA, 23 nt, Homo sapiens
  source 1..23: mol_type = genomic RNA; note = target sequence position 2854-2876
agaaagatgtgagataacttact

SEQ ID NO: 95 - RNA, 23 nt, Homo sapiens
  source 1..23: mol_type = genomic RNA; note = target sequence position 2887-2909
ctccagaaaaatacgtatgttta

SEQ ID NO: 96 - RNA, 23 nt, Homo sapiens
  source 1..23: mol_type = genomic RNA; note = homo sapiens target sequence position 2888-2910
tccagaaaaatacgtatgtttaa

SEQ ID NO: 97 - RNA, 23 nt, Homo sapiens
  source 1..23: mol_type = genomic RNA; note = target sequence position 2889-2911
ccagaaaaatacgtatgtttaaa

SEQ ID NO: 98 - RNA, 23 nt, Homo sapiens
  source 1..23: mol_type = genomic RNA; note = target sequence position 2890-2912
cagaaaaatacgtatgtttaaaa

SEQ ID NO: 99 - RNA, 23 nt, Homo sapiens
  source 1..23: mol_type = genomic RNA; note = target sequence position 2951-2973
ctaaaattgactgtactgtttaa

SEQ ID NO: 100 - RNA, 23 nt, Homo sapiens
  source 1..23: mol_type = genomic RNA; note = target sequence position 2952-2974
taaaattgactgtactgtttaac

SEQ ID NO: 101 - RNA, 23 nt, Homo sapiens
  source 1..23: mol_type = genomic RNA; note = target sequence position 2953-2975
aaaattgactgtactgtttaact

SEQ ID NO: 102 - RNA, 23 nt, Homo sapiens
  source 1..23: mol_type = genomic RNA; note = target sequence position 2988-3010
gaggctaaacacagtttgttttt

SEQ ID NO: 103 - RNA, 23 nt, Homo sapiens
  source 1..23: mol_type = genomic RNA; note = homo sapiens target sequence position 2989-3011
aggctaaacacagtttgtttttc

SEQ ID NO: 104 - RNA, 23 nt, Homo sapiens
  source 1..23: mating_type = target sequence position 2990-3012; mol_type = genomic RNA
ggctaaacacagtttgtttttct

SEQ ID NO: 105 - RNA, 23 nt, Homo sapiens
  source 1..23: mol_type = genomic RNA; note = target sequence position 2995-3017
aacacagtttgtttttcttgtaa

SEQ ID NO: 106 - RNA, 23 nt, Homo sapiens
  source 1..23: mol_type = genomic RNA; note = target sequence position 2996-3018
acacagtttgtttttcttgtaat

SEQ ID NO: 107 - RNA, 23 nt, Homo sapiens
  source 1..23: mol_type = genomic RNA; note = target sequence position 2997-3019
cacagtttgtttttcttgtaatc

SEQ ID NO: 108 - RNA, 23 nt, Homo sapiens
  source 1..23: mol_type = genomic RNA; note = target sequence position 3012-3034
ttgtaatcacttttcatgttaaa

SEQ ID NO: 109 - RNA, 23 nt, Homo sapiens
  source 1..23: mol_type = genomic RNA; note = target sequence position 3013-3035
tgtaatcacttttcatgttaaaa

SEQ ID NO: 110 - RNA, 23 nt, Homo sapiens
  source 1..23: mol_type = genomic RNA; note = homo sapiens target sequence position 3014-3036
gtaatcacttttcatgttaaaat

SEQ ID NO: 111 - RNA, 23 nt, Homo sapiens
  source 1..23: mol_type = genomic RNA; note = target sequence position 3016-3038
aatcacttttcatgttaaaataa

SEQ ID NO: 112 - RNA, 23 nt, Homo sapiens
  source 1..23: mol_type = genomic RNA; note = target sequence position 3018-3040
tcacttttcatgttaaaataatc

SEQ ID NO: 113 - RNA, 23 nt, Homo sapiens
  source 1..23: mol_type = genomic RNA; note = target sequence position 3041-3063
agcattcaaattgtatgctttct

SEQ ID NO: 114 - RNA, 23 nt, Homo sapiens
  source 1..23: mol_type = genomic RNA; note = target sequence position 3093-3115
tgctcgtaaggaaacattttatg

SEQ ID NO: 115 - RNA, 23 nt, Homo sapiens
  source 1..23: mol_type = genomic RNA; note = target sequence position 3097-3119
cgtaaggaaacattttatgtatt

SEQ ID NO: 116 - RNA, 23 nt, Homo sapiens
  source 1..23: mol_type = genomic RNA; note = target sequence position 3100-3122
aaggaaacattttatgtattaaa

SEQ ID NO: 117 - RNA, 23 nt, Homo sapiens
  source 1..23: mol_type = genomic RNA; note = target sequence position 3104-3126
aaacattttatgtattaaaataa

SEQ ID NO: 118 - RNA, 23 nt, Homo sapiens
  source 1..23: mol_type = genomic RNA; note = target sequence position 3121-3143
aaataaactgttccttgataaaa

SEQ ID NO: 119 - RNA, 21 nt, synthetic construct
  source 1..21: mol_type = other RNA; note = RNA oligo guide, target sequence position 1297-1319
aaaatatggctactaagatca

SEQ ID NO: 120 - RNA, 21 nt, synthetic construct
  source 1..21: mol_type = other RNA; note = RNA oligo guide, target sequence position 1323-1345
acaataatcgaaaaatcccgt

SEQ ID NO: 121 - RNA, 21 nt, synthetic construct
  source 1..21: mol_type = other RNA; note = RNA oligo guide, target sequence position 1916-1938
atattggtctcccaatttttg

SEQ ID NO: 122 - RNA, 21 nt, synthetic construct
  source 1..21: mol_type = other RNA; note = RNA oligo guide, target sequence position 1944-1966
tatcctatgctttttttggag

SEQ ID NO: 123 - RNA, 21 nt, synthetic construct
  source 1..21: mol_type = other RNA; note = RNA oligo guide, target sequence position 1945-1967
atatcctatgctttttttgga

SEQ ID NO: 124 - RNA, 21 nt, synthetic construct
  source 1..21: mol_type = other RNA; note = RNA oligo guide, target sequence position 2003-2025
aacatgaagataacaaatccc

SEQ ID NO: 125 - RNA, 21 nt, synthetic construct
  source 1..21: mol_type = other RNA; note = RNA oligo guide, target sequence position 2006-2028
aaaaacatgaagataacaaat

SEQ ID NO: 126 - RNA, 21 nt, synthetic construct
  source 1..21: mol_type = other RNA; note = synthetic RNA oligo guide, target sequence position 2019-2041
aaacgaattgaggaaaaacat

SEQ ID NO: 127 - RNA, 21 nt, synthetic construct
  source 1..21: mol_type = other RNA; note = RNA oligo guide, target sequence position 2094-2116
aatatcagctaaaattagcaa

SEQ ID NO: 128 - RNA, 21 nt, synthetic construct
  source 1..21: mol_type = other RNA; note = RNA oligo guide, target sequence position 2096-2118
tgaatatcagctaaaattagc

SEQ ID NO: 129 - RNA, 21 nt, synthetic construct
  source 1..21: mol_type = other RNA; note = RNA oligo guide, target sequence position 2105-2127
tcaaaatcatgaatatcagct

SEQ ID NO: 130 - RNA, 21 nt, synthetic construct
  source 1..21: mol_type = other RNA; note = RNA oligo guide, target sequence position 2111-2133
attatctcaaaatcatgaata

SEQ ID NO: 131 - RNA, 21 nt, synthetic construct
  source 1..21: mol_type = other RNA; note = RNA oligo guide, target sequence position 2112-2134
aattatctcaaaatcatgaat

SEQ ID NO: 132 - RNA, 21 nt, synthetic construct
  source 1..21: mol_type = other RNA; note = RNA oligo guide, target sequence position 2115-2137
tagaattatctcaaaatcatg

SEQ ID NO: 133 - RNA, 21 nt, synthetic construct
  source 1..21: mol_type = other RNA; note = RNA oligo guide, target sequence position 2205-2227
aatagatggatgtctattaat

SEQ ID NO: 134 - RNA, 21 nt, synthetic construct
  source 1..21: mol_type = other RNA; note = RNA oligo guide, target sequence position 2626-2648
aatagatggatgtctattaat

SEQ ID NO: 135 - RNA, 21 nt, synthetic construct
  source 1..21: mol_type = other RNA; note = RNA oligo guide, target sequence position 2854-2876
taagttatctcacatctttct

SEQ ID NO: 136 - RNA, 21 nt, synthetic construct
  source 1..21: mol_type = other RNA; note = RNA oligo guide, target sequence position 2887-2909
aacatacgtatttttctggag

SEQ ID NO: 137 - RNA, 21 nt, synthetic construct
  source 1..21: mol_type = other RNA; note = RNA oligo guide, target sequence position 2888-2910
aaacatacgtatttttctgga

SEQ ID NO: 138 - RNA, 21 nt, synthetic construct
  source 1..21: mol_type = other RNA; note = RNA oligo guide, target sequence position 2889-2911
taaacatacgtatttttctgg

SEQ ID NO: 139 - RNA, 21 nt, synthetic construct
  source 1..21: mol_type = other RNA; note = RNA oligo guide, target sequence position 2890-2912
ttaaacatacgtatttttctg

SEQ ID NO: 140 - RNA, 21 nt, synthetic construct
  source 1..21: mol_type = other RNA; note = RNA oligo guide, target sequence position 2951-2973
aaacagtacagtcaattttag

SEQ ID NO: 141 - RNA, 21 nt, synthetic construct
  source 1..21: mol_type = other RNA; note = RNA oligo guide, target sequence position 2952-2974
taaacagtacagtcaatttta

SEQ ID NO: 142 - RNA, 21 nt, synthetic construct
  source 1..21: mol_type = other RNA; note = RNA oligo guide, target sequence position 2953-2975
ttaaacagtacagtcaatttt

SEQ ID NO: 143 - RNA, 21 nt, synthetic construct
  source 1..21: mol_type = other RNA; note = RNA oligo guide, target sequence position 2988-3010
aaacaaactgtgtttagcctc

SEQ ID NO: 144 - RNA, 21 nt, synthetic construct
  source 1..21: mol_type = other RNA; note = RNA oligo guide, target sequence position 2989-3011
aaaacaaactgtgtttagcct

SEQ ID NO: 145 - RNA, 21 nt, synthetic construct
  source 1..21: mol_type = other RNA; note = RNA oligo guide, target sequence position 2990-3012
aaaaacaaactgtgtttagcc

SEQ ID NO: 146 - RNA, 21 nt, synthetic construct
  source 1..21: mol_type = other RNA; note = RNA oligo guide, target sequence position 2995-3017
acaagaaaaacaaactgtgtt

SEQ ID NO: 147 - RNA, 21 nt, synthetic construct
  source 1..21: mol_type = other RNA; note = RNA oligo guide, target sequence position 2996-3018
tacaagaaaaacaaactgtgt

SEQ ID NO: 148 - RNA, 21 nt, synthetic construct
  source 1..21: mol_type = other RNA; note = RNA oligo guide, target sequence position 2997-3019
ttacaagaaaaacaaactgtg

SEQ ID NO: 149 - RNA, 21 nt, synthetic construct
  source 1..21: mol_type = other RNA; note = RNA oligo guide, target sequence position 3012-3034
taacatgaaaagtgattacaa

SEQ ID NO: 150 - RNA, 21 nt, synthetic construct
  source 1..21: mol_type = other RNA; note = RNA oligo guide, target sequence position 3013-3035
ttaacatgaaaagtgattaca

SEQ ID NO: 151 - RNA, 21 nt, synthetic construct
  source 1..21: mol_type = other RNA; note = RNA oligo guide, target sequence position 3014-3036
tttaacatgaaaagtgattac

SEQ ID NO: 152 - RNA, 21 nt, synthetic construct
  source 1..21: mol_type = other RNA; note = RNA oligo guide, target sequence position 3016-3038
attttaacatgaaaagtgatt

SEQ ID NO: 153 - RNA, 21 nt, synthetic construct
  source 1..21: mol_type = other RNA; note = RNA oligo guide, target sequence position 3018-3040
ttattttaacatgaaaagtga

SEQ ID NO: 154 - RNA, 21 nt, synthetic construct
  source 1..21: mol_type = other RNA; note = RNA oligo guide, target sequence position 3041-3063
aaagcatacaatttgaatgct

SEQ ID NO: 155 - RNA, 21 nt, synthetic construct
  source 1..21: mol_type = other RNA; note = RNA oligo guide, target sequence position 3093-3115
taaaatgtttccttacgagca

SEQ ID NO: 156 - RNA, 21 nt, synthetic construct
  source 1..21: mol_type = other RNA; note = RNA oligo guide, target sequence position 3097-3119
tacataaaatgtttccttacg

SEQ ID NO: 157 - RNA, 21 nt, synthetic construct
  source 1..21: mol_type = other RNA; note = RNA oligo guide, target sequence position 3100-3122
taatacataaaatgtttcctt

SEQ ID NO: 158 - RNA, 21 nt, synthetic construct
  source 1..21: mol_type = other RNA; note = RNA oligo guide, target sequence position 3104-3126
attttaatacataaaatgttt

SEQ ID NO: 159 - RNA, 21 nt, synthetic construct
  source 1..21: mol_type = other RNA; note = RNA oligo guide, target sequence position 3121-3143
ttatcaaggaacagtttattt

SEQ ID NO: 160 - RNA, 21 nt, synthetic construct
  source 1..21: mol_type = other RNA; note = passenger, target sequence position 1297-1319
atcttagtagccatattttca

SEQ ID NO: 161 - RNA, 21 nt, synthetic construct
  source 1..21: mol_type = other RNA; note = passenger, target sequence position 1323-1345
gggatttttcgattattgtgc

SEQ ID NO: 162 - RNA, 21 nt, synthetic construct
  source 1..21: mol_type = other RNA; note = passenger, target sequence position 1916-1938
aaaattgggagaccaatatcc

SEQ ID NO: 163 - RNA, 21 nt, synthetic construct
  source 1..21: mol_type = other RNA; note = passenger, target sequence position 1944-1966
ccaaaaaaagcataggatatc

SEQ ID NO: 164 - RNA, 21 nt, synthetic construct
  source 1..21: mol_type = other RNA; note = passenger, target sequence position 1945-1967
caaaaaaagcataggatatct

SEQ ID NO: 165 - RNA, 21 nt, synthetic construct
  source 1..21: mol_type = other RNA; note = passenger, target sequence position 2003-2025
gatttgttatcttcatgtttt

SEQ ID NO: 166 - RNA, 21 nt, synthetic construct
  source 1..21: mol_type = other RNA; note = passenger, target sequence position 2006-2028
ttgttatcttcatgtttttcc

SEQ ID NO: 167 - RNA, 21 nt, synthetic construct
  source 1..21: mol_type = other RNA; note = passenger, target sequence position 2019-2041
gtttttcctcaattcgtttgt

SEQ ID NO: 168 - RNA, 21 nt, synthetic construct
  source 1..21: mol_type = other RNA; note = passenger, target sequence position 2094-2116
gctaattttagctgatattca

SEQ ID NO: 169 - RNA, 21 nt, synthetic construct
  source 1..21: mol_type = other RNA; note = passenger, target sequence position 2096-2118
taattttagctgatattcatg

SEQ ID NO: 170 - RNA, 21 nt, synthetic construct
  source 1..21: mol_type = other RNA; note = passenger, target sequence position 2105-2127
ctgatattcatgattttgaga

SEQ ID NO: 171 - RNA, 21 nt, synthetic construct
  source 1..21: mol_type = other RNA; note = passenger, target sequence position 2111-2133
ttcatgattttgagataattc

SEQ ID NO: 172 - RNA, 21 nt, synthetic construct
  source 1..21: mol_type = other RNA; note = passenger, target sequence position 2112-2134
tcatgattttgagataattct

SEQ ID NO: 173 - RNA, 21 nt, synthetic construct
  source 1..21: mol_type = other RNA; note = passenger, target sequence position 2115-2137
tgattttgagataattctaca

SEQ ID NO: 174 - RNA, 21 nt, synthetic construct
  source 1..21: mol_type = other RNA; note = passenger, target sequence position 2205-2227
ccacttaatagaatatgttgg

SEQ ID NO: 175 - RNA, 21 nt, synthetic construct
  source 1..21: mol_type = other RNA; note = passenger, target sequence position 2626-2648
taatagacatccatctattgc

SEQ ID NO: 176 - RNA, 21 nt, synthetic construct
  source 1..21: mol_type = other RNA; note = passenger, target sequence position 2854-2876
aaagatgtgagataacttact

SEQ ID NO: 177 - RNA, 21 nt, synthetic construct
  source 1..21: mol_type = other RNA; note = passenger, target sequence position 2887-2909
ccagaaaaatacgtatgttta

SEQ ID NO: 178 - RNA, 21 nt, synthetic construct
  source 1..21: mol_type = other RNA; note = passenger, target sequence position 2888-2910
cagaaaaatacgtatgtttaa

SEQ ID NO: 179 - RNA, 21 nt, synthetic construct
  source 1..21: mol_type = other RNA; note = passenger, target sequence position 2889-2911
agaaaaatacgtatgtttaaa

SEQ ID NO: 180 - RNA, 21 nt, synthetic construct
  source 1..21: mol_type = other RNA; note = passenger, target sequence position 2890-2912
gaaaaatacgtatgtttaaaa

SEQ ID NO: 181 - RNA, 21 nt, synthetic construct
  source 1..21: mol_type = other RNA; note = passenger, target sequence position 2951-2973
aaaattgactgtactgtttaa

SEQ ID NO: 182 - RNA, 21 nt, synthetic construct
  source 1..21: mating_type = passenger, target sequence position 2952-2974; mol_type = other RNA
aaattgactgtactgtttaac

SEQ ID NO: 183 - RNA, 21 nt, synthetic construct
  source 1..21: mol_type = other RNA; note = passenger, target sequence position 2953-2975
aattgactgtactgtttaact

SEQ ID NO: 184 - RNA, 21 nt, synthetic construct
  source 1..21: mol_type = other RNA; note = passenger, target sequence position 2988-3010
ggctaaacacagtttgttttt

SEQ ID NO: 185 - RNA, 21 nt, synthetic construct
  source 1..21: mol_type = other RNA; note = passenger, target sequence position 2989-3011
gctaaacacagtttgtttttc

SEQ ID NO: 186 - RNA, 21 nt, synthetic construct
  source 1..21: mol_type = other RNA; note = passenger, target sequence position 2990-3012
ctaaacacagtttgtttttct

SEQ ID NO: 187 - RNA, 21 nt, synthetic construct
  source 1..21: mol_type = other RNA; note = passenger, target sequence position 2995-3017
cacagtttgtttttcttgtaa

SEQ ID NO: 188 - RNA, 21 nt, synthetic construct
  source 1..21: mol_type = other RNA; note = passenger, target sequence position 2996-3018
acagtttgtttttcttgtaat

SEQ ID NO: 189 - RNA, 21 nt, synthetic construct
  source 1..21: mol_type = other RNA; note = passenger, target sequence position 2997-3019
cagtttgtttttcttgtaatc

SEQ ID NO: 190 - RNA, 21 nt, synthetic construct
  source 1..21: mol_type = other RNA; note = passenger, target sequence position 3012-3034
gtaatcacttttcatgttaaa

SEQ ID NO: 191 - RNA, 21 nt, synthetic construct
  source 1..21: mol_type = other RNA; note = passenger, target sequence position 3013-3035
taatctcttttcatgttaaaa

SEQ ID NO: 192 - RNA, 21 nt, synthetic construct
  source 1..21: mol_type = other RNA; note = passenger, target sequence position 3014-3036
aatcacttttcatgttaaaat

SEQ ID NO: 193 - RNA, 21 nt, synthetic construct
  source 1..21: mol_type = other RNA; note = passenger, target sequence position 3016-3038
tcacttttcatgttaaaataa

SEQ ID NO: 194 - RNA, 21 nt, synthetic construct
  source 1..21: mol_type = other RNA; note = passenger, target sequence position 3018-3040
acttttcatgttaaaataatc

SEQ ID NO: 195 - RNA, 21 nt, synthetic construct
  source 1..21: mol_type = other RNA; note = passenger, target sequence position 3041-3063
cattcaaattgtatgctttct

SEQ ID NO: 196 - RNA, 21 nt, synthetic construct
  source 1..21: mol_type = other RNA; note = passenger, target sequence position 3093-3115
ctcgtaaggaaacattttatg

SEQ ID NO: 197 - RNA, 21 nt, synthetic construct
  source 1..21: mol_type = other RNA; note = passenger, target sequence position 3097-3119
taaggaaacattttatgtatt

SEQ ID NO: 198 - RNA, 21 nt, synthetic construct
  source 1..21: mol_type = other RNA; note = passenger, target sequence position 3100-3122
ggaaacattttatgtattaaa

SEQ ID NO: 199 - RNA, 21 nt, synthetic construct
  source 1..21: mol_type = other RNA; note = passenger, target sequence position 3104-3126
acattttatgtattaaaataa

SEQ ID NO: 200 - RNA, 21 nt, synthetic construct
  source 1..21: mol_type = other RNA; note = passenger, target sequence position 3121-3143
ataaactgttccttgataaaa

(19) United States
(12) Patent Application Publication
(10) Pub. No.: US 2026/0286365 A1
(43) Pub. Date: Sep. 24, 2026

(54) OCA2 MODULATORS, COMPOSITIONS AND USES THEREOF

(71) Applicant: AURORA PHARMACEUTICALS LTD, Petah Tikva (IL)

(72) Inventor: Ran GREENBERG, Petah Tikva (IL)

(21) Appl. No.: 19/469,629

(22) PCT Filed: Mar. 28, 2024
(86) PCT No.: PCT/IL2024/050326

§ 371 Date: Sep. 26, 2025

Related U.S. Application Data
(60) Provisional application No. 63/492,640, filed on Mar. 28, 2023.

(51) Int. Cl. C12N 15/113; A61K 8/60; A61K 31/713; A61P 27/02; A61Q 90/00

(52) U.S. Cl. CPC C12N 15/1138; A61K 8/606; A61K 31/713; A61P 27/02; A61Q 90/00; C12N 2310/11; C12N 2310/14

(57) ABSTRACT

The present disclosure relates to methods for modifying the eye color of a subject via modulation of the levels and/or activity and/or stability of the OCA2 protein. The present disclosure further relates to at least one OCA2 protein modulating agent or any compositions comprising the same and uses thereof.

### FIELD OF THE INVENTION

The present disclosure relates to cosmetic applications. More specifically, the present disclosure relates to modulation of the Oculocutaneous Albinism 2 (OCA2) protein in a subject, for example for modifying the eye color of said subject.

### BACKGROUND ART

References considered to be relevant as background to the presently disclosed subject matter are listed below:
[1] Eiberg H, et al. (2008) Hum Genet. 2008 March; 123 (2): 177-87.
[2] Suarez P, et al (2021). Sci Rep. 11 (1): 22530.
[3] Park, S., Morya, V. K., Nguyen, D. H. et al. (2015) Mol Cell Biochem 403, 61-71.
[4] WO2021146668
[5] U.S. Pat. No. 9,744,237

Acknowledgement of the above references herein is not to be inferred as meaning that these are in any way relevant to the patentability of the presently disclosed subject matter.

### BACKGROUND OF THE INVENTION

Many people desire to change or enhance the color of their eyes, skin or hair for cosmetic reasons. For example, there is a large market for products that change the color of the eyes. However, traditional methods such as contact lenses, implants and laser surgery have not been effective. Additionally, traditional methods for skin and hair color treatment have been problematic, requiring multiple applications of potentially toxic products.

There is also a growing problem among those taking glaucoma medication, which can cause the eyes to darken, resulting in an unsightly appearance. As the population ages and the prevalence of glaucoma increases, more people are experiencing this issue. Additionally, asymmetry caused by congenital or acquired heterochromia (for example due to Horner's or Fuch's iridocyclitis) could be corrected by treating the contralateral, or the darker eye.

Eye color is determined by a number of intrinsic factors, including the amount of melanin in the iris, the type of melanin (eumelanin/pheomelanin) and ratio, the number of melanosomes and their maturity level, the distance between melanosomes and the nucleus, and the number of fibroblasts and collagen in the iris. Color is also given by the cornea, as its removal reveals a difference in color vividness.

Melanocytes are cells that are found in various tissues in the body, their main role is to regulate the amount of pigmentation in the tissue through synthesis of melanin. They are mainly found in the eyes (e.g. iris), hair, and skin.

Melanin is a brown pigment, its abundance is the iris gives brown eyes their color. The blue color of blue eyes, however, is not due to a pigment, but rather to an optical phenomenon similar to that which produces blue skies. Light in the wavelengths of the entire spectrum of colors enters the eye. The long waves (yellow and red) continue relatively unhindered until they are absorbed into the back of the iris, and the short waves—in the absence of melanin, the blues tend to scatter more frequently (Rayleigh scattering), a random phenomenon that occurs more frequently with shorter waves.

Melanin is produced in specialized organelles called melanosomes. It is essential for melanosomes to maintain a proper inner pH in order to mature and become heavily pigmented. As a result of the absence of the protein OCA2, melanosomes are not functional, are incapable of maturing, and pigment production in the tissues is significantly reduced. The significant majority of blue-eyed individuals have a reduced amount of OCA2 protein, resulting in immature melanosomes in their iris stroma, which results in reduced pigmentation.

Melanosome maturation occurs in the cytoplasm and is generally divided into four distinct stages, with stage 4 considered mature. Several proteins are required for melanosome maturation. In brown eyes, melanosome organelles are present mainly at stage 4 of maturation, while in blue eyes, most of the melanosomes are at stages I-II and a few are at stage III, with virtually zero melanosomes at stage IV.

The eye color of most people with bright eyes is due to an ancestral mutation that occurred in the HERC2 gene on chromosome 15 [1]. Although many mutations and genetic changes contribute to the wide range of colors in human skin, hair, and eyes, the decrease in expression of the OCA2 gene is responsible for the majority of light eyes. Mutations in the two genes, OCA2 itself and HERC2 which acts also as an OCA2 transcription promoter, are responsible for approximately 90% of an individual's eye color phenotype (bright/brown) [2]. It was previously shown that the inhibition of pink-eyed dilution protein (P-protein, produced by the OCA2 gene) can change the melanosomal morphology, types and their respective numbers and may play a role in the control of melanin synthesis in murine skin cancer cells [3].

WO2021146668 [4] discloses a method for changing a color of a subject's iris, through genetic manipulation inducing death of melanocytes in a stroma of the iris.

U.S. Pat. No. 9,744,237 [5] discloses methods and systems for lightening the color of the iris through administration of a tyrosinase inhibitor.

There is a need for a safe and effective way to change the color of the eyes, as well as a solution for reversing eye discoloration caused by glaucoma medication. Additionally, a quickly penetrating topical eye medication is needed that can overcome the barriers that currently inhibit the penetration of active ingredients in the eye, such as precorneal and corneal factors, or alternatively, through the scleral-conjunctival pathways or other pathways found between in any form of administration (subconjunctival, intravitreal, intracameral, subretinal, systemic, or others) to the target tissue, the iris. There is also a need for a delivery system for cosmetic or therapeutic agents that can target pigmented tissues of the iris and effectively change the color or deliver treatment without adversely affecting the patient.

### SUMMARY OF THE INVENTION

In a first aspect, the present disclosure provides a method of modifying the eye color of a subject comprising administering to said subject an effective amount of at least one Oculocutaneous Albinism 2 (OCA2) protein modulating agent, or any vehicle, matrix, nano- or micro-particle thereof or any composition comprising the same.

In another aspect, the present disclosure provides at least one Oculocutaneous Albinism 2 (OCA2) protein modulating agent or any vehicle, matrix, nano- or micro-particle thereof or any composition comprising the same for use in a method of modifying the eye color of a subject comprising administering to said subject an effective amount of said at least one OCA2 protein modulating agent.

In a further aspect, the present disclosure provides a composition for modifying the eye color of a subject comprising at least one Oculocutaneous Albinism 2 (OCA2) protein modulating agent.

In a further aspect, the present disclosure provides a pharmaceutical composition comprising least one Oculocutaneous Albinism 2 (OCA2) protein modulating agent and at least one of pharmaceutically acceptable carrier/s, diluent/s and/or excipient/s.

In yet another aspect, the present disclosure provides a nucleic acid molecule for modifying the eye color of a subject comprising at least one Oculocutaneous Albinism 2 (OCA2) protein modulating agent.

In a further aspect, the present disclosure provides a method of treating a disease/disorder in a subject in need thereof comprising administering to said subject an effective amount of at least one Oculocutaneous Albinism 2 (OCA2) protein modulating agent or any vehicle, matrix, nano- or micro-particle thereof or any composition comprising the same.

In a yet additional aspect, the present disclosure provides at least one Oculocutaneous Albinism 2 (OCA2) protein modulating agent or any vehicle, matrix, nano- or micro-particle thereof or any composition comprising the same for use in a method of treating a disease/disorder in a subject in need thereof comprising administering to said subject an effective amount of said at least one OCA2 protein modulating agent.

### BRIEF DESCRIPTION OF THE DRAWINGS

The patent or application file contains at least one drawing executed in color. Copies of this patent or patent application publication with color drawings will be provided by the Office upon request and payment of the necessary fee.

In order to better understand the subject matter that is disclosed herein and to exemplify how it may be carried out in practice, embodiments will now be described, by way of non-limiting example only, with reference to the accompanying drawings, in which:

FIG. 1: Melanin Assessment.

Rational Calibration for Melanin Assessment (OD at 360 nm) measured directly on B16-F10 cells at different concentrations 5 days after seeding.

FIG. 2: OCA2 expression.

OCA2 expression of B16-F10 cells following transfection with OCA2 siRNA #1, #2 or #3 (as denoted by SEQ ID Nos: 70 to 75), at 20 nM or 50 nM. Abbreviations: “neg” is negative control, “lipo only” (lipofectamine only), and “UN” (untransfected).

FIG. 3: Melanin absorbance.

Melanin absorbance at OD 360 nm following transfection with OCA2 siRNA #1, #2 or #3 (as denoted by SEQ ID Nos: 70 to 75), of B16-F10 cells at different concentrations.

FIG. 4: Iris Viability assay.

Viability Assay of Murine Iris Explants Cultured in Three Types of Media: A, B, and C. Iris-4 and Iris-12 were non-viable from the first day and remained so throughout the study. The remaining samples retained their viability with only minor changes observed.

FIG. 5A-5B: Ex-Vivo Iris Transfection Visualization.

FIG. 5A: Picture of an iris sample transfected with fluorophore-conjugated control siRNA (green) and counterstained with Hoechst (blue), demonstrating the co-localization of the siRNA within cellular structures. The red square indicating the specific region situated around the 6 o'clock position of the iris.

FIG. 5B: Zoom in of the red square represented in FIG. 5A.

The complete morphology of the iris is depicted in FIG. 5A.

FIG. 6: SOX10 expression of iris samples following SOX10 siRNA or scrambled siRNA transfection. Each group contains a pool of 2 iris samples.

FIG. 7A-7B: Effect of exposure to a light source.

Comparative Analysis of Hair Samples Post Three-Week Interval.

FIG. 7A: Control Group Without Light Exposure.

FIG. 7B: Experimental Group Subjected to Light Exposure.

This figure showcases the differentiation in pigmentation between female donor's light brown hair and male donor's darker hair, following a three-week period under distinct conditions.

FIG. 8A-8D: Depigmentation in ex vivo vital iris samples-macroscopic photographs before and after OCA2-siRNA treatment.

FIG. 8A: Pre-treatment image of an iris sample.

FIG. 8B: Post-treatment image of an iris sample from the OCA2-siRNA group.

FIG. 8C: Pre-treatment image of an iris sample from the control group, baseline.

FIG. 8D: Post-treatment image of an iris sample from the control group.

FIG. 9: Iris color saturation.

Graph showing average iris color-saturation value in the treated group versus the control group. The difference in background was corrected for all images, by subtracting the color-saturation value of the background from the color-saturation value of the iris.

FIG. 10A-10C: Comparative Microscopic Analysis of Iris Tissues Post-Treatment.

FIG. 10A: Picture of an of a microscopic analysis of an iris tissue following treatment with OCA2-siRNA and photobleaching.

FIG. 10B: Picture of an of a microscopic analysis of an iris tissue following treatment with scrambled siRNA and photobleaching.

FIG. 10C: Picture of an of a microscopic analysis of an iris tissue without any treatment or photobleaching.

### DETAILED DESCRIPTION OF THE INVENTION

The currently proposed solutions for modifying the eye color are not satisfying and include severe side effects, non-aesthetic results that are far from natural, and inconvenient uses.

The present disclosure aims to address the unmet need for changing eye color in a safe and effective manner and relates to modulation of the level of OCA2 protein in an eye's subject. A natural aesthetic look and perceptible change in the eye color is achieved by the proposed compositions and methods disclosed herein.

In a first aspect, the present disclosure provides a method of modifying the eye color of a subject. The method comprising administering to said subject an effective amount of at least one Oculocutaneous Albinism 2 (OCA2) protein modulating agent, or any vehicle, matrix, nano- or micro-particle thereof or any composition comprising the same.

As used herein, the Oculocutaneous Albinism 2 (OCA2) gene relates to a gene that encodes the OCA2 protein also named P-protein. In some embodiments, the OCA2 is the human OCA2. In yet some further embodiments, the human OCA2 gene is located on the Chromosome 15 (Location 15q12-q13.1). In some embodiments, the human OCA2 mRNA comprises the nucleic acid sequence as denoted by the accession number NM_000275.3. In some embodiments, the human OCA2 mRNA comprises the nucleic acid sequence as denoted by SEQ ID NO: 65. Still further, in some embodiments, and the human OCA2 mRNA encodes the OCA2 protein having the accession number NP 000266.2. In some embodiments, the human OCA2 protein may comprise the amino acid sequence as denoted by SEQ ID NO: 66.

As used herein, a modulating agent of OCA2 protein relates to any agent that may modify (either increasing or decreasing) the level of active OCA2 protein either directly i.e. by acting on the OCA2 protein itself or by acting on the OCA2 gene and its transcription. In yet some further embodiments, the modulating agent may act indirectly at any target molecule or any sequence that controls and/or affects the expression and/or activity and/or stability and/or tissue distribution and/or cellular localization of OCA2. In some embodiments, the modulating agent may be modified to increase its efficiency and reduce its potential adverse effects. In some embodiments, the modulating agent may include auxiliary materials such as nanoparticle envelopes with defined roles to help deliver the drug through anatomical any physiological barriers, improve its stability within the tissue, introduce the drug into the cells and increase the rate of endosomal escape events. The material may include, but is not limited to, lipids, polymers, etc. In some embodiments the modulating agent may be conjugated with a ligand or molecule capable of anchoring the envelope to the target cell. In some embodiments, the modulating agent does not activate the immune system, e.g., it does not increase cytokine levels, such as TNF-alpha or IFN-alpha levels, for example, when measured by an assay, such as an in vitro.

In some embodiments, the OCA2 protein modulating agent may be at least one of a nucleic acid molecule, an aptamer, a peptide, a peptidomimetic, an immunological agent, a small molecule, a proteolysis targeting chimera (PROTAC), glycans and any combinations thereof. In some embodiments, the eye color is modified to become blue, green, gray, azure, yellow, brown, amber, hazel, black, violet, red, or any color of the visible spectrum.

In some embodiments, the OCA2 protein modulating agent may be a nucleic acid molecule. As used herein, the term “nucleic acid”, “nucleic acid sequence”, “oligonucleotide” or “polynucleotide” and “nucleic acid molecule” refers to polymers of nucleotides, and includes but is not limited to deoxyribonucleic acid (DNA), ribonucleic acid (RNA), DNA/RNA hybrids including polynucleotide chains of regularly and/or irregularly alternating deoxyribosyl moieties and ribosyl moieties (i.e., wherein alternate nucleotide units have an —OH, then and —H, then an —OH, then an —H, and so on at the 2′ position of a sugar moiety), xeno nucleic acid (XNA) (e.g., PNA etc.) and modifications of these kinds of polynucleotides, wherein the attachment of various entities or moieties to the nucleotide units at any position are included. The terms should also be understood to include, as equivalents, analogs of either RNA or DNA made from nucleotide analogs, and, as applicable to the embodiment being described, single-stranded (such as sense or antisense) and double-stranded polynucleotides. Preparation of nucleic acids is well known in the art.

In some embodiments, the OCA2 protein modulating agent may be a PROTAC. PROTACs as used herein, are typically designed with three parts: (1) a ligand/molecule that binds to and/or modulates ubiquitin ligases; (2) a binding moiety that targets and recruits the protein of interest for proteolysis, e.g. OCA2; and (3) a linker that links the two molecules together. PROTACs thus function by allowing the ligand/molecule to bind to the ubiquitin ligases, thereby recruiting the target of protein of interest to the ligase for ubiquitination and ultimately proteolysis and degradation.

Other names can however be found in the literature: e.g., specific and non-genetic IAP-dependent protein erasers (SNIPER); degrader; degronimids; PROteolysis TArgeting Peptide (PROTAP); Protein Degradation Probe (PDP). PROTACs hijack the catalytic activity of ubiquitin E3 ligases to mediate proteasome dependent degradation of selected protein of interest (POI), by bringing the ligase and POI into close spatial proximity and initiating the poly-ubiquitination process.

In some embodiments, the OCA2 protein modulating agent may be an aptamer. As used herein, an aptamer is a short single-stranded DNA or RNA molecule, typically ranging from about 20 to 100 nucleotides in length, that can bind specifically to a target molecule e.g. the OCA2 protein with high affinity and specificity. Aptamers are often referred to as “chemical antibodies” because they can recognize and bind to specific targets, such as proteins, small molecules, or even whole cells, with a binding affinity comparable to or sometimes even exceeding that of antibodies. Aptamers with high affinity and specificity for the target protein, e.g. OCA2, are selected using SELEX (Systematic Evolution of Ligands by Exponential Enrichment) or a similar process. During SELEX, a large pool of random sequence nucleic acids (DNA or RNA) is exposed to the target protein, and sequences that bind tightly to the protein are retained while non-binding sequences are washed away.

In some embodiments, the OCA2 protein modulating agent may be a peptide. In some further embodiments, the OCA2 protein modulating agent may be a peptidomimetic. As used herein, a peptidomimetic is a synthetic compound that mimics the structure and/or function of a peptide (a short chain of amino acids), designed to interact with biological molecules such as proteins, e.g. the OCA2 protein.

In some embodiments, the OCA2 protein modulating agent may be an immunological agent. As used herein, an immunological agent typically refers to a molecule designed to interact with a protein of interest i.e. the OCA2 protein derived from the immune system, either directly or indirectly. These agents may include but are not limited to antibodies, antibody derivatives, nanobodies or the like.

In some embodiments, the OCA2 protein modulating agent may be a small molecule. As used herein, a small molecule refers to a chemical compound with a relatively low molecular weight that is designed to interact specifically with a particular protein target, i.e. the OCA2 protein. These small molecules are often referred to as “ligands” when they bind to a protein. In some embodiments, said at least one OCA2 modulating agent is at least one gene editing agent adapted for modulating the expression and/or activity and/or stability of OCA2.

As used herein, a gene editing agent is able to modulate the expression and/or activity of OCA2 by various mechanism. For example, the gene-editing agent may modulate OCA2 expression and/or activity by genetic modification of the chromosomal DNA that may result in knockdown cell, organism and the like. In addition, the gene editing agent may modulate OCA2 expression and/or activity by a transient/temporary modification (transient knockdown).

The gene editing agent in accordance with the compositions and methods of the invention may be in some embodiments a natural occurring compound (such as an enzyme, short DNA or RNA oligonucleotide), a synthetic compound or an artificial compound. In some embodiments, the at least one gene editing agent may be an oligonucleotide. Such oligonucleotide can bind to an active gene or any transcripts thereof and cause decreased expression by, for example blocking of transcription for example in the case of gene-binding by Anti-Sense Oligonucleotides (ASO), the degradation of the mRNA transcript e.g. by small interfering RNA (siRNA) or RNase-H dependent antisense, or through the blocking of either mRNA translation, pre-mRNA splicing sites, or nuclease cleavage sites used for maturation of other functional RNAs, including miRNA (e.g. by morpholino oligos or other RNase-H independent antisense oligonucleotide).

In some embodiments, said OCA2 modulating agent may be a gene editing such as any one of a Clustered Regularly Interspersed Short Palindromic Repeats (CRISPR)/Cas system agent, a Transcription Activator-Like Effector Nuclease (TALEN) agent or a Zinc-Finger Nucleases (ZFN) agent.

In some embodiments, the OCA2 modulating agent may be a Clustered Regularly Interspersed Short Palindromic Repeats (CRISPR)/Cas system agent. As used herein, the Clustered Regularly Interspaced Short Palindromic Repeats (CRISPR) system is a bacterial immune system that has been modified for genome engineering.

CRISPR-Cas systems fall into two classes. Class 1 systems use a complex of multiple Cas proteins to degrade foreign nucleic acids. Class 2 systems use a single large Cas protein for the same purpose. More specifically, Class 1 may be divided into types I, III, and IV and class 2 may be divided into types II, V, and VI.

The CRISPR-Cas system has evolved in prokaryotes to protect against phage attack and undesired plasmid replication by targeting foreign DNA or RNA. The CRISPR-Cas system, targets DNA molecules based on short homologous DNA sequences, called spacers that exist between repeats. These spacers guide CRISPR-associated (Cas) proteins to matching (and/or complementary) sequences within the target DNA (e.g., foreign DNA), called proto-spacers, which are subsequently cleaved. The spacers can be rationally designed to target any target DNA sequence, for example, within the OCA2 gene sequence. Thus, in yet some specific embodiments, the at least one cas gene used in the methods and compositions of the invention may be at least one cas gene of type II CRISPR system. CRISPR type II system as used herein requires the inclusion of two essential components: a “guide” RNA (gRNA) and a non-specific CRISPR-associated endonuclease (Cas9). The gRNA is a short synthetic RNA composed of a “scaffold” sequence necessary for Cas9-binding (also named tracrRNA) and about 20 nucleotide long “spacer” or “targeting” sequence, which defines the genomic target to be modified. Guide RNA (gRNA), as used herein refers to a synthetic fusion of the endogenous tracrRNA with a targeting sequence (also named crRNA), providing both scaffolding/binding ability for Cas9 nuclease and targeting specificity.

In some embodiments, the OCA2 modulating agent may be a TALEN agent. As used herein, TALEN are restriction enzymes that can be engineered to cut specific sequences of DNA. They are made by fusing a TAL effector DNA-binding domain to a DNA cleavage domain of a nuclease. More specifically, TALENs are artificial endonucleases designed by fusing the DNA-binding domain (multiples of nearly identical repeats each comprised of about 34 amino acids) obtained from TAL (transcription activator-like) effector (TALE) protein to the cleavage domain of the FokI endonuclease. Each TALE repeat independently recognizes its corresponding nucleotide (nt) base with two variable residues [termed the repeat variable di-residues (RVDs)] such that the repeats linearly represent the nucleotide sequence of the binding site.

In some embodiments, the OCA2 modulating agent may be a ZFN agent. ZFNs are artificial restriction enzymes generated by fusing a zinc finger DNA-binding domain to a DNA-cleavage domain. Zinc finger domains can be engineered to target specific desired DNA sequences and this enables zinc-finger nucleases to target unique sequences within complex genomes. More specifically, the ZFNs are artificial endonucleases that have been generated by combining a small zinc finger (ZF; about 30 amino acids) DNA-binding/recognition domain (Cys₂His₂) to a type IIS nonspecific DNA-cleavage domain from the FokI restriction enzyme. However, the cleavage activity of the FokI endonuclease demands dimerization. As a ZF module recognizes a 3 bp sequence, there is a requirement for multiple fingers in each ZFN monomer for recognizing and binding to longer DNA target sequences.

In some embodiments, said at least one OCA2 modulating agent comprises at least one nucleic acid molecule.

In some embodiments, said nucleic acid molecule comprise at least one nucleic acid sequence as denoted by SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199 or 200.

In some specific embodiments, said nucleic acid molecule comprises at least one of a single stranded RNA (ssRNA), a single stranded DNA (ssDNA), a double stranded DNA (dsDNA), a double stranded RNA (dsRNA), nucleic acid molecule having at least one modified nucleotide/s and any combinations thereof.

In some more specific embodiments, said nucleic acid molecule comprises at least one of a small interfering RNA (siRNA), an antisense oligonucleotide (ASO), a guide RNA, a short hairpin RNA (shRNA), microRNA (miRNA), Peptide-Nucleic Acid (PNA) and locked nucleic acid (LNA).

In some embodiments, the OCA2 modulating agent of the invention acts via RNA silencing. As used herein, the phrase “RNA silencing” refers to a group of regulatory mechanisms e.g. RNA interference (RNAi), transcriptional gene silencing (TGS), post-transcriptional gene silencing (PTGS), quelling, co-suppression, and translational repression mediated by RNA molecules which result in the inhibition or “silencing” of the expression of a corresponding protein-coding gene or RNA sequence. In certain embodiments, the RNA silencing agent is capable of preventing complete processing (e.g., the full translation and/or expression) of an mRNA molecule through a post-transcriptional silencing mechanism. RNA silencing agents include noncoding RNA molecules, for example RNA duplexes comprising paired strands, as well as precursor RNAs from which such small non-coding RNAs can be generated. Exemplary RNA silencing agents include but are not limited to dsRNAs such as siRNAs, miRNAs, shRNAs, piwi-interacting RNAs (piRNAs), long non-coding RNAs (lncRNAs), antisense oligonucleotides (ASOs), ribozymes, DNA-directed RNAs (ddRNAs), small activating RNAs (saRNAs), CRISPR RNAs (crRNAs), and Dicer-substrate siRNAs (dsiRNAs). In some embodiments, the RNA silencing agent is capable of inducing RNA interference. In other embodiments, the RNA silencing agent is capable of mediating translational repression. More specifically, the nucleic acid agent according to the invention may encode an “antisense RNA”, which is a single strand RNA (ssRNA) molecule that is complementary to an mRNA strand of a specific target gene product. Antisense RNA may inhibit the translation of a complementary mRNA by base-pairing to it and physically obstructing the translation machinery. By “complementary” it is meant the ability of polynucleotides to form base pairs with one another. Base pairs are typically formed by hydrogen bonds between nucleotide units in antiparallel polynucleotide strands. Complementary polynucleotide strands can base pair in the Watson-Crick manner (e.g., A to T, A to U, C to G), or in any other manner that allows for the formation of duplexes.

In yet some specific embodiments, the nucleic acid modulating agents of the present disclosure may encode an RNA, specifically, dsRNA molecule that participates in RNA interference. RNA interference (RNAi), as indicated above, is a general conserved eukaryotic pathway which down regulates gene expression in a sequence specific manner. It is the process of sequence-specific, post-transcriptional gene silencing in animals and plants, initiated by siRNA that is homologous in its duplex region to the sequence of the silenced gene. Gene silencing is induced and maintained by the presence of partly or perfectly double-stranded RNA (dsRNA). The silenced genes may be endogenous or exogenous to the organism, integrated into a chromosome or present in a transfection vector that is not integrated into the genome. The expression of the target OCA2 gene is either completely or partially inhibited.

More specifically, the dsRNA modulating agents encompassed by the present disclosure may be selected from the group consisting of small interfering RNA (siRNA), MicroRNA (miRNA), short hairpin RNA (shRNA), PIWI interacting RNAs (piRNAs).

In some further specific embodiments, OCA2 modulating agent may be a nucleic acid molecule comprising at least one siRNA molecule. In some embodiments, the siRNA molecule targets at least one sequence coding or controlling directly or indirectly the levels and/or activity and/or stability of OCA2.

Specifically as used herein, a siRNA molecule relates to a molecule comprising two strands: an antisense strand being the “guide” for the mRNA and a sense strand not actively involved in gene silencing. The duration of time that siRNA molecules remain in tissue is determined by their degradation by nuclease enzymes. In some embodiments, siRNAs may be synthesized with a modified sugar phosphate backbone, which inhibits the activity of these enzymes thereby enabling the siRNA molecules to remain in tissues for a longer period of time. In some specific embodiments, the siRNA comprises blunt ends.

In some particular embodiments, the siRNA molecule may target at least one region in the OCA2 transcript or mRNA. In some specific embodiments, the siRNA molecule may target at least one region in the human OCA2 transcript or mRNA. In some embodiments, said human OCA2 transcript or mRNA may comprise the nucleic acid molecule as denoted by 65. In some embodiments, the siRNA molecule may target the nucleotide region positioned at 375-420 or 1270-1370 or 1500-1600 or 1900-2400 or 2610-3143 in the OCA2 mRNA transcript. Other suitable regions that may be targeted by the siRNA of the present disclosure are described in Example 5 below.

In some embodiments, the siRNA molecule may comprise at least one nucleic acid sequence as denoted by SEQ ID NO: 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199 or 200.

In some alternative embodiments, said nucleic acid molecule comprises at least one ASO. In some further embodiments, as used herein, Antisense Oligonucleotide (ASOs) are single-stranded, synthetic RNA (or DNA) sequences, which may be highly-modified, and are designed to selectively bind via complementary base-pairing to RNA which encodes the gene of interest. The binding of the ASO to the target can trigger a range of outcomes when bound to its complementary target. These outcomes range from altering mRNA processing to degrading the target transcript. Synthesized ASOs can: bind to complementary sequences in pre-mRNA, altering the recruitment of splicing factors to the molecule, regulating splicing events; bind to mature mRNA and prevent its attachment to the ribosome, blocking protein translation or can recruit RNase H to the target transcript, which will then be degraded.

In some particular embodiments, the nucleic acid agent modulating agent (e.g. ASO or siRNA) can be synthesized by standard methods known in the art as further discussed below, e.g., by use of an automated DNA/RNA synthesizer, such as are commercially available from, for example, Biosearch, Applied Biosystems, Inc. The dsRNA compounds of the invention may be prepared using a two-step procedure. First, the individual strands of the dsRNA compounds are prepared separately. Then, the component strands are annealed. The individual strands of the dsRNA compounds can be prepared using solution-phase or solid-phase organic synthesis or both. Organic synthesis offers the advantage that the oligonucleotide strands comprising unnatural or modified nucleotides can be easily prepared.

In some further embodiments, the single-stranded oligonucleotides of the invention can be prepared using solution-phase or solid-phase organic synthesis or both.

The embodiments described above can include nucleic acid molecule agents such as siRNA, ASO or other types of RNA interference that have at least one strand with at least 17 nucleotides in length due to the nature of the oligonucleotide sequences provided herein. The shorter duplexes described above, minus only a few nucleotides on one or both ends, can be expected to be equally effective as compared to the nucleic acid molecule agent. In other words, nucleic acid molecule agents that contain a sequence of at least 15, 16, 17, 18, 19, 20, or more contiguous nucleotides that are derived from the sequences described herein but differ in their ability to inhibit the expression of the OCA2 gene by not more than 30% inhibition from a nucleic acid molecule agent that contains the full sequence, are contemplated to be within the scope of the present invention. In some specific embodiments, the nucleic acid molecule agents may comprise 19 nucleotides.

In the present invention, nucleic acid molecule agents may contain one or more mismatches to the target sequence. One embodiment describes a nucleic acid molecule agent that contains no more than three mismatches. If the antisense strand of a RNA therapeutic agent includes mismatches with a target sequence, it is preferable that the mismatch not be located at the center of the complementarity region. Instead, the mismatch should be located within the last five nucleotides of either the 5′ end or the 3′ end of the complementarity region. It is possible to determine whether a RNA therapeutic agent containing a mismatch to a target sequence is effective in inhibiting the expression of an OCA2 gene using the methods described herein or methods known in the art. If the particular complementarity region in an OCA2 gene is known to be subject to polymorphic sequence variation within the population, then it is pertinent to consider the efficacy of a RNA therapeutic agent with mismatches in inhibiting the expression of OCA2.

Despite some of the sequences in Table 2 being described are not modified and/or conjugated sequences, the RNA of the RNA therapeutic agent of the invention, e.g., dsRNA may include any one of these sequences described in Table 2, which have not been modified, or not conjugated, or have been modified and/or conjugated differently than described therein.

In one embodiment, the invention uses a nucleic acid molecule agent that is unmodified and does not include chemical modifications or conjugations known to those skilled in the art or described herein. Another embodiment of the invention involves chemical modification of the nucleic acid molecule agent to improve stability or other beneficial properties (resistance to nuclease, increased uptake by endocytosis, increased tissue retention, increased escape rates of endosomes, increased reservoir of nucleic acid molecules near melanin, increased silencing efficiency, more suitable pharmacokinetics profile, better distribution to the anterior chamber and/or the iris, RISC uptake of the guide strand, among others). In certain embodiments of the invention, substantially all of the nucleotides of the nucleic acid molecule agent are modified. As an example, nearly all of the nucleotides of the sense strand are modified nucleotides, and/or virtually all of the nucleotides of the antisense strand are modified nucleotides, and/or substantially all of the nucleotides of both the sense and antisense strands are modified nucleotides.

In another embodiment of the invention, all of the nucleotides of the nucleic acid molecule agent are modified. In this case, all of the nucleotides of the sense strand are modified nucleotides, and/or all of the nucleotides of the antisense strand are modified nucleotides, and/or all of the nucleotides of both the sense strand and antisense strand are modified nucleotides.

The nucleic acid molecule agents of the invention are capable of being synthesized and/or modified according to known methods. It includes, for example, end modifications, such as 5′-end modifications (phosphorylation, conjugation, inverted linkages) or 3′-end modifications (conjugation, DNA nucleotides, inverted linkages, etc.). Base modifications, for example, substitution with stabilizing bases, destabilizing bases, bases that base pair with an expanded repertoire of partners, or removal of bases (abasic nucleotides). In addition, sugar modifications such as modification at the 2′ or 4′ positions (e.g., 2′-O-methyl(2′-OMe), 2′-Fluoro (2′-F), Locked nucleic acid (LNA), 5-Methylcytidine (5mC), Unlocked nucleic acid (UNA), 2′-O-(2-methoxyethyl) (2′-MOE)) or sugar replacement are available, as well as modifications to the backbone, such as modification or replacement of the phosphodiester links.

Examples of nucleic acid molecule compounds useful for the embodiments described herein include, but are not limited to, nucleic acid molecules containing modified backbones or nucleic acid molecules lacking natural internucleoside linkages. Among the nucleic acid molecules with modified backbones are those that lack a phosphorus atom. As mentioned in the art, modified nucleic acid molecules without a phosphorus atom in their internucleoside backbone can also be considered to be oligonucleosides for the purposes of this specification. A modified nucleic acid molecules may contain a phosphorus atom in its internucleoside backbone in some embodiments. Modified nucleic acid molecule backbones include, for example, phosphorothioates, chiral phosphorothioates, phosphorodithioates, phosphotriesters, aminoalkylphosphotriesters, methyl and other alkyl phosphonates, such as 3′-alkylene phosphonates and chiral phosphonates, phosphinates, phosphoramidates including 3′-amino phosphoramidates. As well as aminoalkylphosphoramidates, thionophosphoramidates, thionoalkylphosphonates, thionoalkylphosphotriesters, and boranophosphates having normal 3′-5′ linkages, 2′-5′-linked analogs of these, and those having inverted polarity wherein the adjacent pairs of nucleoside units are linked 3′-5′ to 5′-3′ or 2′-5′ to 5′-2′. Various salts, mixed salts and free acid forms are also covered.

In other embodiments of the invention, suitable mimetics of RNA can be used for creating RNA interference. In these embodiments, both sugar and internucleoside linkages, i.e., the backbone, of nucleotide units are replaced with novel groups. The base units are maintained for hybridization with an appropriate nucleic acid target compound. A peptide nucleic acid (PNA) that may be used as the OCA2 modulating agent in accordance with some embodiments of the present disclosure, is an RNA-mimetic compound with excellent hybridization properties that has an oligomeric structure. Instead of phosphodiester linkages, PNAs are made up of peptide bonds, and their nucleobases are attached directly to an N-methyl glycine backbone. Its properties make the PNA more stable and less susceptible to degradation, making it an effective method for RNA interference. The nucleobases are retained and bonded directly or indirectly to the aza nitrogen atoms in the amide portion of the backbone. Preparation of PNA compounds is known in the art. There are additional PNA compounds suitable for use in the iRNAs of the invention, for example, Nielsen et al., Science, 1991, 254, 1497-1500.

In another embodiment, the at least one modulating agent targets at least one region in the OCA2 transcript.

In yet another embodiment, the at least one modulating agent targets at least one region in the OCA2 gene.

In some additional embodiments, the at least one modulating agent targets several regions e.g. between 2 to 10, e.g. between 2 to 9, e.g. between 2 to 8, e.g. between 2 to 7, e.g. between 2 to 6, e.g. between 2 to 5 or e.g. between 2 to 4 regions of the OCA2 gene or OCA2 transcript.

In certain embodiment, the at least one modulating agent targets at least one region in the OCA2 transcript that is susceptible to RNA-Induced Silencing Complex (RISC)-mediated cleavage.

In yet another embodiment, the at least one modulating agent targets at least one region in the OCA2 transcript via inhibition of translation by blocking ribosome access to the transcript (mRNA).

In some other embodiments, the at least one modulating agent targets at least one region in the OCA2 transcript that is susceptible to RNA-Induced Silencing Complex (RISC)-mediated cleavage, which may include degradation of the transcript (mRNA) by the argonaute proteins of the RISC and by the exoribonuclease Xrn1.

In some further embodiments, the at least one modulating agent targets at least one region in the OCA2 transcript via RNase H-mediated cleavage. In some specific embodiment in which the at least one modulating agent is an ASO, it can form a duplex with the mRNA target, which recruits the RNase H enzyme to cleave the mRNA.

In some further embodiments, the at least one modulating agent targets at least one region in the OCA2 transcript via Steric hindrance. In some specific embodiment in which the at least one modulating agent is an ASO, it can bind to the mRNA and block access to ribosomes, preventing translation.

In some further embodiments, the at least one modulating agent targets at least one region in the OCA2 transcript via Splicing modulation. In some specific embodiment in which the at least one modulating agent is an ASO, it can alter the splicing of pre-mRNA, leading to the exclusion or inclusion of specific exons in the final mRNA.

In some further embodiments, the at least one modulating agent targets at least one region in the OCA2 transcript via Activation of nonsense-mediated decay. In some specific embodiment in which the at least one modulating agent is an ASO, it can target mRNA with premature termination codons (PTCs), triggering nonsense-mediated decay and leading to degradation of the mRNA.

In some embodiments, said modulating agent may increase the expression and/or activity and/or stability of OCA2. In accordance with some embodiments of the present disclosure, the OCA2 modulating agent may increase the expression and/or activity of OCA2 e.g., in a cell, tissue, anterior chamber of the eye or any other compartment of the eye subject by at least about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least about 99% or more.

In certain embodiments, said OCA2 modulating agent affects the levels of melanin in the iris tissue of said subject. In some embodiments, said OCA2 modulating agent affects the levels of eumelanin. In some other embodiments, said OCA2 modulating agent affects the levels of pheomelanin. In some other embodiments, said OCA2 modulating agent affects the average maturation of melanosomes.

In some embodiments, said modulating agent inhibits and/or reduces the expression and/or activity and/or stability of OCA2. Thus, in accordance with some embodiments of the present disclosure, the OCA2 modulating agent may reduce/inhibit the expression and/or activity of OCA2 e.g., in a cell (e.g. iris stromal cells), tissue, anterior chamber of the eye or any other compartment of the eye subject by at least about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least about 99% or more.

It is to be understood that, where provided, percentage values such as, for example, 10%, 50%, 120%, 500%, etc., are interchangeable with “fold change” values, i.e., 0.1, 0.5, 1.2, 5, etc., respectively. Reduction/Inhibition or increasing as mentioned above may be assessed by a decrease or increase in an absolute or relative level of one or more variables that are associated with expression and/or activity, and/or stability of OCA2 compared with a control level. The control level may be any type of control that is utilized in the art, e.g., a pre-dose baseline level, or a level determined from a similar subject, cell, group of cells or sample that is untreated or treated with a control (such as, e.g., buffer only control or inactive agent control), specifically, any in cell (e.g. iris stromal cells), that was not exposed to or contacted with the OCA2 modulating agent of the present disclosure. The reduction in the expression can be assessed using any method currently available in the field. In order to determine the reduction of OCA2 expression, one can, for example, quantify the level of mRNA expression of the protein using methods common to one with ordinary skill in the art, such as Northern blotting and qRT-PCR. Additionally, one can measure the level of protein in OCA2 (also called P-protein) by employing methods routine to one with ordinary skill in the art which include, but are not limited to, Western blotting, immunological techniques. Alternatively, one can assess whether successful inhibition has occurred by analyzing the inhibition downstream phenotypical effects e.g., melanosome maturity, pigmentation density and pH within the melanosomes.

In certain embodiments, said inhibition reduces the levels of melanin in the iris tissue of said subject. As used herein, “Melanin” is a natural pigment produced by specialized cells known as melanocytes. It serves various functions throughout the body, including protection against UV radiation and determining the coloration of tissues like skin, hair, and eyes. In the eyes, melanin plays a crucial role in the iris, the colored part of the eye. The amount and distribution of melanin within the iris contribute to the spectrum of eye colors observed in individuals. Specifically, there are two primary types of melanin involved in eye coloration eumelanin and pheomelanin. Eumelanin is responsible for darker pigmentation and contributes to brown and black hues in the eyes. Pheomelanin is associated with lighter pigmentation and contributes to red and yellow tones, often seen in lighter-colored eyes such as blue or green.

In some embodiments, said inhibition reduces the levels of eumelanin. In some other embodiments, said inhibition reduces the levels of pheomelanin.

In accordance with some embodiments of the present disclosure, the OCA2 modulating agent may reduce/inhibit the levels of melanin, specifically eumelanin and/or pheomelanin in a cell (e.g. iris stromal cells), tissue, anterior chamber of the eye or any other compartment of the eye subject by at least about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least about 99% or more.

In some further embodiments, said inhibition reduces the average maturation of melanosomes. As used herein, “melanosomes” are specialized cellular organelles found within melanocytes, the cells responsible for producing melanin. These organelles are crucial for the synthesis, storage, and transportation of melanin within the cell and its eventual transfer to surrounding tissues. In the eye, melanin production occurs within melanosomes located in the melanocytes of the iris. These melanosomes produce either eumelanin or pheomelanin, depending on genetic and environmental factors. The type and quantity of melanin synthesized within these melanosomes determine the coloration of the iris. Melanosomes in the iris can vary in size, shape, and distribution among individuals, influencing the intensity and shade of eye color. For instance, individuals with darker eye colors typically have more densely packed and larger melanosomes containing higher levels of eumelanin, whereas those with lighter eye colors have fewer and smaller melanosomes, often containing more pheomelanin.

In some embodiments, said inhibition reduces the average maturation of melanosomes at least about 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least about 99% or more.

In some specific embodiments, said inhibition occurs in the iris stroma.

In some further specific embodiments, said OCA2 modulating agent reduces the expression and/or activity and/or stability of OCA2, said reduction results in lightening the eye color of said subject.

In some more specific embodiments, said at least one OCA2 modulating agent targets the iris stroma of said subject.

As used herein, the iris stroma is the connective tissue layer of the iris, located between the anterior and posterior layers of the iris. It consists of fibroblasts, collagen fibers, blood vessels, and pigmented cells called stromal melanocytes. The iris stroma gives the iris its structural integrity and contributes to its color, primarily through the distribution of pigmented cells. In some embodiments, said OCA2 modulating agent targets the iris stromal melanocytes of said subject.

In some embodiments, said at least one OCA2 modulating agent targets the cells of at least one of the pigmented epithelium, the uvea, the choroid, the ciliary body, the Retinal Pigment Epithelium (RPE) and/or the Iris Pigment Epithelium (IPE).

In some embodiments, said at least one OCA2 modulating agent targets the cells of the Pigmented Epithelium. As used herein, the cells of the Pigmented Epithelium form the pigmented layer of the retina, which is located between the neural retina and the choroid. The pigmented epithelium helps in the absorption of excess light, thereby preventing reflection and scattering of light within the eye. These cells also play a role in the regeneration of visual pigments in the photoreceptor cells.

In some embodiments, said at least one OCA2 modulating agent targets the cells of the Uvea. The uvea is the middle layer of the eye, consisting of the iris, ciliary body, and choroid. Cells of the uvea include melanocytes, fibroblasts, and blood vessel-related cells. Melanocytes provide pigmentation to the iris and choroid, contributing to eye color and light absorption. Fibroblasts contribute to the structural integrity of the uveal tract. Blood vessel-related cells include endothelial cells and pericytes, which are involved in maintaining vascular function.

In some embodiments, said at least one OCA2 modulating agent targets the cells of the Choroid. The choroid is a vascular layer located between the retina and the sclera (the white outer layer of the eye). Choroidal cells include melanocytes, fibroblasts, endothelial cells, and pericytes. Melanocytes provide pigmentation to the choroid, aiding in light absorption and reducing intraocular scatter. Endothelial cells and pericytes are involved in regulating blood flow and nutrient exchange within the choroidal vasculature.

In some embodiments, said at least one OCA2 modulating agent targets the cells of the Ciliary Body (CB). The ciliary body is a muscular structure located behind the iris. Cells of the ciliary body include epithelial cells, muscle cells, and secretory cells. Epithelial cells are involved in the production of aqueous humor, a fluid that nourishes the cornea and lens. Muscle cells control the shape of the lens for accommodation (focusing at different distances). Secretory cells produce components of the aqueous humor and regulate its secretion and outflow.

In some embodiments, said at least one OCA2 modulating agent targets the cells of the Retinal Pigment Epithelium (RPE). The retinal pigment epithelium is a single layer of cells located between the neural retina and the choroid. RPE cells provide metabolic support to the photoreceptor cells (rods and cones) of the retina. They phagocytose shed photoreceptor outer segments and recycle visual pigments. RPE cells also contribute to the blood-retinal barrier and regulate the transport of nutrients and waste products between the retina and the choroid.

In some embodiments, said at least one OCA2 modulating agent targets the cells of the Iris Pigment Epithelium (IPE). The IPE is a layer of pigmented cells located on the posterior surface of the iris, facing the anterior chamber of the eye. These cells contribute to the color of the iris and help in controlling the amount of light entering the eye by regulating the size of the pupil. IPE cells also play a role in the production and circulation of aqueous humor, the fluid that fills the anterior chamber of the eye.

In some embodiments, said at least one modulating agent penetrates through the cornea and/or enter the anterior chamber of the eye.

In some embodiments, said at least one modulating agent escapes destruction by the endo-lysosomal system.

In some embodiments, said at least one modulating agent does not activate the immune system.

In some embodiments, said at least one modulating agent is encapsulated within or coated with at least one nano-carrier.

In some embodiments, said at least one OCA2 protein modulating agent comprises at least one nucleic acid molecule, or any vector or host cell comprising the same.

In some further embodiments, the methods of the present disclosure may further comprise the step of exposing said subject to at least one light source. In some embodiments, said step may accelerate lightening of the eye color.

In some embodiments, any form of light exposure may be employed such as from the sun, ambient sources, or therapeutic lights for any suitable time interval, at any suitable intensity and/or at any suitable waive length, such that the melanin levels are reduced in the iris tissue of the subject. Additional steps may be included that may accelerate melanin breakdown and/or may be coupled with the inhibition of new melanin production.

In some further embodiments, the method of the present disclosure may further comprise the step of administering additional pharmacological agent for inhibiting melanin production. In some further embodiments, the method of the present disclosure may further comprise the step of requiring from said subject having prolonged water fasting.

Autophagy can facilitate the breakdown of proteins, including those in melanosomes, leading to a decrease in melanosome and melanin levels in cells. Thus, in some embodiments, autophagy may be accelerated or enhanced. Notably, behaviors such as extended water fasting can promote autophagy, and genetic predispositions may also play a role. It may be possible to genetically modify subjects to exhibit increased autophagy, employing these modified animals as models. In some embodiments, these models are utilized, or subjects are required to undergo prolonged periods of water fasting, to achieve the described effects.

In some embodiments, the administration of an effective amount of the at least one Oculocutaneous Albinism 2 (OCA2) protein modulating agent may be repeated several times, for example from 2 to 100 times and/or at different intervals, for example from 1 day to about 1 week.

In some embodiments, the administration of an effective amount of the at least one Oculocutaneous Albinism 2 (OCA2) protein modulating agent may be repeated about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 times or about 60, 70, 80, 90, 100, 110, 120, 130, 140, 150, 160, 170, 180, 190 or 200 times.

In still some further embodiments, the Oculocutaneous Albinism 2 (OCA2) protein modulating agent or any composition/s and any components thereof may be applied as a single daily dose or multiple daily doses, preferably, every 1 to 60, or 1 to 365 days. It is specifically contemplated that such application may be carried out once, twice, thrice, four times, five times or six times daily, or may be performed once daily, once every 2 days, once every 3 days, once every 4 days, once every 5 days, once every 6 days, once every week, two weeks, three weeks, four weeks, a month, two months or even several months to a year. The application of the composition/of the invention or of any component thereof may last up to a day, two days, three days, four days, five days, six days, a week, two weeks, three weeks, four weeks, a month, two months three months or even more e.g. for the entire lifespan of a subject.

In some embodiments, said repetition may be required due to repigmentation of the eye tissue of said subject.

In some embodiments, said at least one OCA2 modulating agent is administered via ocular administration.

In certain embodiments, said at least one OCA2 modulating agent is administered via topical administration. In some embodiments, said topical administration is into the eye of said subject.

In some embodiments, said at least one OCA2 modulating agent is comprised in an eye drop formulation.

In some alternative embodiments, said at least one OCA2 modulating agent is administered into the anterior chamber of the eye.

In some embodiments, the method of the present disclosure is for cosmetic purposes.

In some embodiments, the subject is a healthy subject.

The term “healthy” refers to a state of being free/not suffering from illness, injury, or disease/disorder. In some specific embodiment, a healthy subject refers to a subject that does not suffer from an eye disorder. In some specific embodiment, a healthy subject refers to a subject that does not suffer from cancer. In some more specific embodiment, a healthy subject refers to a subject that does not suffer from melanocytic cancer.

In some embodiments the present disclosure provides non-therapeutic methods. In yet some further embodiments, the present disclosure provides cosmetic methods.

In some alternative embodiments, the subject suffers from a disease.

In some specific embodiments, said disease may be any one of hyperpigmentation ocular disorders; hyperpigmentation; side effects due to glaucoma medications; acquired heterochromia (e.g., due to Horner syndrome or Fuchs Heterochromic Iridocyclitis) and congenital heterochromia (e.g., due to genetic syndromes such as Waardenburg Syndrome); Bilateral Diffuse Uveal Melanocytic Proliferation (BDUMP); ocular nevi (e.g., choroidal nevus); Pigment Dispersion Syndrome; ocular melanoma; complexion-associated melanosis; primary acquired melanosis; Freckle; Lisch nodule; melanocytoma; ocular/oculodermal melanocytosis (e.g., Nevus of Ota); and benign ocular growths or any pigmentary ocular disorder, as well as pigmentary disorders presenting in the eye but are due to systemic conditions such as Addison's disease.

In a further aspect, the present disclosure provides at least one Oculocutaneous Albinism 2 (OCA2) protein modulating agent or any vehicle, matrix, nano- or micro-particle thereof or any composition comprising the same for use in a method of modifying the eye color of a subject comprising administering to said subject an effective amount of said at least one OCA2 protein modulating agent.

It should be noted that all the previously described embodiments with respect to the method of modifying the eye color aspect defined above are also applicable to the second aspect of the present disclosure relating to the OCA2 modulating agent for use in accordance with the present disclosure, as well as for any aspect of the present disclosure.

In a third aspect, the present disclosure provides a composition for modifying the eye color of a subject comprising at least one Oculocutaneous Albinism 2 (OCA2) protein modulating agent. Optionally, the composition of the present disclosure may further comprise any carrier diluent and any suitable excipient.

In some embodiments, said modulating agent is at least one gene editing agent adapted for modulating the expression and/or activity and/or stability of OCA2.

In some embodiments, said at least one modulating agent comprises at least one nucleic acid molecule.

In some embodiments, said nucleic acid molecule comprises at least one of a single stranded DNA (ssDNA), a single stranded RNA (ssRNA), a double stranded DNA (dsDNA), a double stranded RNA (dsRNA), nucleic acid molecule having at least one modified nucleotide/s and any combinations thereof.

In some specific embodiments, said nucleic acid molecule comprises at least one of a small interfering RNA (siRNA), an antisense oligonucleotide (ASO), a guide RNA, a short hairpin RNA (shRNA), microRNA (miRNA), Peptide-Nucleic Acid (PNA) and locked nucleic acid (LNA). In yet some more specific embodiments, said nucleic acid molecule comprises at least one siRNA molecule, wherein said siRNA molecule targets at least on sequence coding or controlling directly or indirectly the levels and/or activity and/or stability of OCA2. In some alternative embodiments, said nucleic acid molecule comprises at least one ASO. In certain embodiments, the at least one OCA2 modulating agent targets at least one region in the OCA2 transcript.

In other embodiments, the at least one modulating agent targets at least one region in the OCA2 gene.

In certain specific embodiments, the at least one modulating agent targets at least one region in the OCA2 transcript that is susceptible to RNA-Induced Silencing Complex (RISC)-mediated cleavage.

In certain embodiments, said OCA2 modulating agent inhibits and/or reduces the expression and/or activity and/or stability of OCA2.

In some embodiments, said inhibition reduces the levels of melanin in the iris tissue of said subject.

In some specific embodiments, said inhibition reduces the average maturation of melanosomes. In some further specific embodiments, said inhibition reduces the average maturation of melanosomes in the iris stroma.

In some embodiments, said OCA2 modulating agent reduces the expression and/or activity and/or stability of OCA2, said reduction results in lightening the eye color of said subject.

In some specific embodiments, said at least one OCA2 modulating agent targets the iris stroma of said subject.

In some further embodiments, said at least one OCA2 modulating agent targets the stromal melanocytes of said subject.

In some other embodiments, said at least one modulating agent is able to target the cells of at least one of the pigmented epithelium, the uvea, the choroid, the ciliary body, the Retinal Pigment Epithelium (RPE) and/or the Iris Pigment Epithelium (IPE).

In some embodiments, said at least one modulating agent penetrates through the cornea and enter the anterior chamber of the eye.

In some embodiments, said at least one modulating agent escapes destruction by the endo-lysosomal system.

In some embodiments, said at least one modulating agent does not activate the immune system.

One embodiment of the invention involves administration of an nucleic acid molecule agent e.g. RNA based in its “naked” form or as a “free RNA” e.g. “free siRNA”. A naked nucleic acid molecule agent does not include any pharmaceutical components. An appropriate buffer solution may be used to contain naked nucleic acid molecule agent. e.g. RNA based agent. A buffer solution may be composed of acetate, citrate, prolamine, carbonate, or phosphate, or a combination of these substances (or any other pharmaceutically accepted carrier). The buffer solution in one embodiment is phosphate buffered saline (PBS). It is possible to adjust the pH and osmolarity of the buffer solution that contains the nucleic acid molecule agent. e.g. RNA based agent so that it is suitable for administration to a subject. Benefit of buffer is its ability to maintain a relatively constant pH in solution. Even when large amounts of acid or base are added, the buffer remains effective at stabilizing the nucleic acid molecule agent. e.g. RNA based agent and maintaining its activity. Moreover, the buffer is not toxic and does not adversely affect biological systems. Therefore, it is generally considered to be biocompatible. The buffer is also suitable for production on a large scale.

Alternatively, other embodiments involve delivering the molecule via a drug delivery system. The delivery system may be able to facilitate the passage of the drug through physiological and anatomical barriers like the tear film and the corneal layers or pass through the scleral-conjunctival route, improve its stability within the tissues (reducing the likelihood of nucleic acid molecule agent e.g. RNA based agent degradation by nuclease enzymes), improve the agent uptake by cells (primarily iris stromal melanocytes), and increase the rate of endosomal escape events.

By modifying the nucleic acid molecule agent e.g. RNA based agent or delivering it using a drug delivery system, the nucleic acid molecule agent is prevented from being rapidly degraded in vivo by endo- and exo-nucleases. Modification of the nucleic acid molecule or pharmaceutical carrier can also facilitate targeting of the RNA therapeutic agent composition to the target tissue, increase the likelihood of loading the guide strand into the RISC, improve biodistribution to the target tissue, increase stability to RNase and prevent unwanted off-target effects. In an alternative embodiment, the OCA2 modulating agent e.g. nucleic acid molecule agent can be delivered via carriers e.g. nanoparticles e.g., dendrimers, polymers, liposomes and LNP (e.g. cationic nanoparticles or ionizable lipid nanoparticles).

For example, in the case of RNA based agent, the RNA molecule (negatively charged) can be attached to positively charged cationic delivery systems, thereby facilitating binding and enhancing interactions at the negatively charged cell membrane, resulting in efficient uptake by the cells. An RNA molecule can be bound to cationic lipids, dendrimers, or polymers, or encased within a vesicle or micelle (see e.g., Kim S H., et al. (2008) Journal of Controlled Release 129 (2): 107-116). Furthermore, the formation of vesicles or micelles prevents degradation of the RNA molecule. It is well within the capabilities of one skilled in the art to make and administer cationic-iRNA complexes (see, for instance, Sorensen, D R., et al. (2003) J. Mol. Biol 327:761-766; Verma, U N., et al (2003) Clin. Cancer Res. 9:1291-1300; Arnold, A S et al (2007) J. Hypertens. 25:197-205, hereby incorporated by reference in their entirety).

Ionizable cationic lipids with pKa values below 7 have been developed due to the rapid clearance of charged LNPs from circulation following intravenous injection (see, for example, Rosin et al., Molecular Therapy, vol. 19, no. 12, pages 1286-2200, December 2011). It is possible to load negatively charged molecules such as siRNA and oligonucleotides into LNPs at low pH values (for example, pH 4) when the ionizable lipids display a positive charge. At physiological pH values, LNPs have a low surface charge, allowing them to circulate for a longer period of time unimpeded by immune cells.

In certain embodiments, said at least one OCA2 modulating agent is encapsulated within or coated with at least one nano-carrier. In some embodiment, said nanocarrier is at least one Lipid Nanoparticle (LNP). As used herein, Lipid nanoparticles (LNPs) are colloidal systems composed of lipids and other amphiphilic molecules that self-assemble into nanoscale structures. These nanoparticles are commonly used as delivery systems for various bioactive molecules, such as drugs, genetic materials (like mRNA or DNA), and imaging agents.

In some further specific embodiments, said at least one LNP comprises ionizable lipid nanoparticles.

Among the substances that can be contained within the lipid nanocarrier are ionizable lipids, structural lipids, stabilizing lipids, structured lipids, cationic lipids, and lipids that can reduce immunogenicity. All of these components contribute to the overall functionality of the lipid nanocarrier and can be optimized to enhance its performance and effectiveness as a drug delivery system.

Besides controlling the charge and stability of lipid nanocarriers, the ionizable lipid component may facilitate nanocarrier uptake by target cells and assist in escaping the endosomal-lysosome system by altering the endosomal membrane bilayer configuration to the hexagonal phase. Therefore, the active ingredient, e.g. the RNA based molecule or RNAi (or alternatively DNA based molecule) is able to escape destruction and reach the cytosol, where it is able to exert its effect on its target mRNA (see, for example, Schlich et al., Bioengineering & Translational Medicine 2021 Mar. 20; 6 (2): e10213).

Ionizable lipids in a physiological environment (pH 7.4) are neutral, whereas in an acidic environment, such as the gradually acidifying endosome environment, they are charged. As neutral molecules in the physiologic environment, such as the blood, they are less likely to be quarantined by the immune system due to their neutral molecular structure. Additionally, as endosomal maturation begins and the environment becomes more acidic, the heads of the ionizable lipids turn positive and begin binding with the negative lipids exposed at the endosomal membrane. This binding perturbs the original organization of the endosomal membrane, resulting in the formation of nonbilayer, hexagonal (HII) structures, causing membrane fusion and endosomal disruption, allowing the entrapped nucleic acid to escape.

A number of ionizable cationic lipids have been investigated, including 1,2-dilineoyl-3-dimethylammonium-propane (DLinDAP), 1,2-dilinoleyloxy-3-N,N-dimethylaminopropane (DLinDMA), 1,2-dilinoleyloxy-keto-N,N-dimethyl-3-aminopropane (DLinKDMA), 1,2-dilinoleyl-4-(2-dimethylaminoethyl)-[1,3]-dioxolane (DLinKC2-DMA), and 4-(dimethylamino)-butanoic acid, (10Z,13Z)-1-(9Z,12Z)-9,12-octadecadien-1-yl-10,13-nonadecadien-1-yl ester (DLin-MC3-DMA). Studies have demonstrated that LNP siRNA systems containing these lipids exhibit remarkably better gene silencing properties.

It is structural lipids that provide the basic structure and shape of the lipid nanocarriers. Generally, they consist of a mixture of lipids, such as cholesterol, sterols, and steroids. These components together form a well-defined and stable structure.

Lipids that stabilize lipid nanocarriers prevent aggregation and destabilization during storage or circulation. There are several types of stabilizing lipids, including zwitterionic lipids and phospholipids (e.g., phosphatidyl choline, phosphatidyl ethanolamine, etc.). The examples given here are not intended to be exhaustive.

Nanoparticles for reducing immunogenicity are designed to prevent the immune system from responding to lipid nanocarriers, improving the safety and effectiveness of drug delivery systems. Among the examples are lipids containing immune-suppressive moieties, polymeric compounds and polymer-lipid complexes, as well as pegylated lipids with a polyethylene glycol (PEG) region. It is possible to use any molecular weight for the PEG region. Depending on the embodiment, the PEG region can have a molecular weight of 200, 300, 350, 400, 500, 550, 750, 1000, 1500, 2000, 3000, 3500, 4000 or 5000 Da.

In some cases, additional lipid components may be added in order to enhance the delivery of the OCA2 modulating agent of the invention. A component such as this may be able to reduce the side effects of the drug or increase its effectiveness. Among these are targeting moieties, cationic lipids, and others.

These components may augment the tissue-specificity and enhance drug accumulation at the designated site of action, lipid nanocarriers can be intricately modified with specific targeting moieties. Analogous to the utilization of GalNAc for targeted hepatic delivery, other conjugates that exhibit high affinity for markers uniquely expressed by stromal melanocytes within the ocular milieu may be harnessed to achieve precise ocular targeting. This strategic modification facilitates the directed delivery of therapeutic agents to the iris, thereby potentially amplifying the therapeutic efficacy while concomitantly reducing systemic distribution and associated adverse effects. Through iterative experimentation and optimization processes, both in vitro and in silico, the selection and proportioning of targeting moieties and lipid constituents may be meticulously tailored to meet the exigencies of ocular delivery, ensuring enhanced specificity and efficacy in targeting the stromal melanocytes of the iris. Through trial and error in wet labs and in-silico experiments, the choice of lipids and ratio of lipids may be customized and optimized.

An embodiment of the invention involves the use of OCA2 modulating nucleic acid agent encapsulated in a lipid formulation to form nucleic acid-lipid particles. Particles of the present invention are substantially nontoxic. Nucleic acid-lipid particles and their preparation methods are well described in the art.

In one embodiment, the lipid to drug ratio (mass/mass ratio) (e.g., lipid to dsRNA ratio) is approximately 1:1 to about 50:1.

In some embodiments, another modification of the OCA2 modulating agent of the compositions and methods of the invention involves chemically linking the agent or the delivery system (i.e. the nanoparticle envelope) to one or more ligands, moieties or conjugates that enhance the activity, cellular distribution or cellular uptake of the agent as well as anchoring it to the target cell.

Depending on the embodiment, ligands may alter the distribution, targeting, or lifetime of agents that they are conjugated to. As an example of an embodiment, a ligand provides enhanced affinity for a specific target, e.g., a molecule, cell or cell type, compartment, tissue, organ or region of the body, as opposed to a compound lacking such a ligand. In some embodiments, the ligand will not be involved in duplex pairing in the case of a duplexed nucleic acid.

An example of a ligand could be a cell or tissue targeting agent, such as an antibody, lectin, glycoprotein, lipid, or a lipid-containing molecule. These targeting groups bind to a specific type of cell. Additionally, the ligand can be a substance, such as a drug, that enhances the uptake of the agent into target cells.

In preferred embodiments, the targeting ligand can bind to receptors found on the surface of iridial melanocytes, such as SCF receptors, WNT receptors, EGFR receptors, catecholamine receptors, histamine receptors, prostaglandin receptors, or any other ligand that directs the agent to the iridial melanocytes of a subject.

The ligands may be proteins, e.g., glycoproteins, or peptides, e.g., molecules bound to the co-ligand, or antibodies, e.g., antibodies that bind to a specific cell type, such as melanocytes. Hormones and hormone receptors can also serve as ligands. Additionally, they may also include non-peptidic species, including lipids (such as cholesterol), lectins, carbohydrates, vitamins (such as vitamin E), cofactors, multivalent lactose, amino acids (such as Tyrosine) and others.

Depending on the embodiment, a ligand attached to an agent may function as a pharmacokinetic modulator (PK modulator). There are many types of PK modulators, including lipophiles, bile acids, steroids, phospholipid analogues, peptides, protein binding agents, PEG, vitamins, etc. Examples of PK modulators include cholesterol, fatty acids, cholic acid, lithocholic acid, dialkylglycerides, diacylglycerides, phospholipids, sphingolipids, naproxen, ibuprofen, vitamin E, biotin, etc.

It has been shown that oligonucleotides containing a number of phosphorothioate linkages bind serum protein, thus shorter oligonucleotides (e.g., oligonucleotides of about five, ten, fifteen or twenty bases) that contain multiple phosphorothioate linkages in their backbones can also be employed as ligands (e.g., as PK modulating ligands). Moreover, aptamers that bind serum components (e.g. serum proteins) may also be used as PK modulating ligands in the embodiments described here.

Ligand-conjugated oligonucleotides of the invention can be synthesized using an oligonucleotide with a pendant reactive functionality, for instance, from attaching a linking molecule. These reactive oligonucleotides might directly interact with commercially available ligands, ligands synthesized with protective groups, or ligands bearing a linking moiety. Additionally, Click Chemistry and Site-Specific Conjugation Techniques offer alternative strategies the attachment of ligands. In some embodiments, said at least one OCA2 modulating agent is administered via ocular administration.

In some specific embodiments, said at least one OCA2 modulating agent is administered via topical administration. In some more specific embodiments, said topical administration is into the eye of said subject.

In certain embodiments, said at least one OCA2 modulating agent is comprised in an eye drop formulation.

In some embodiments, the composition according to the present disclosure is for use as a cosmetic composition.

In a further aspect, the present disclosure provides a pharmaceutical composition comprising least one Oculocutaneous Albinism 2 (OCA2) protein modulating agent and at least one of pharmaceutically acceptable carrier/s, diluent/s and/or excipient/s.

In some embodiments, said pharmaceutical composition is as defined the previous aspect of the present disclosure relating to a composition.

Pharmaceutical composition of the present disclosure may include, but is not limited to, solutions, emulsions, and formulations containing carriers (e.g. nano-carriers). Various components can be used to create these compositions, including but not limited to preformed liquids, self-emulsifying solids, and self-emulsifying semisolids. Formulations targeting iridial melanocytes are particularly preferred.

It is possible to prepare the pharmaceutical formulations of the present invention in unit dosage form according to conventional methods well known in the pharmaceutical industry. In such techniques, active ingredients are associated with pharmaceutical carriers or excipients. Formulations are typically prepared by combining the active ingredients uniformly and intimately with liquid carriers or finely divided solid carriers or both, and then shaping them if necessary.

The compositions of the present invention are capable of being formulated into any of a number of dosage forms, such as suspensions in aqueous, non-aqueous, or mixed media. The viscosity of aqueous suspensions can also be increased by substances such as sodium carboxymethylcellulose, sorbitol, and/or dextran. Stabilizers may also be included in the suspension.

In some embodiments, the OCA2 modulating agent or any composition or specific pharmaceutical composition comprising the same in accordance with the present disclosure can be administered and dosed by the methods of the invention, in accordance with good medical practice, systemically, for example intravenously. It should be noted however that the present disclosure may further encompass additional administration modes. In other examples, the agent or any composition or pharmaceutical composition thereof can be introduced to a site by any suitable route including, but not limited to intraocular injection, intracameral, intranasal, topical, oral, subcutaneous, intradermal, intravenous, intramuscular administration and any combinations thereof. In some particular embodiments, the agent may be administered topically via eye drops or eye drop formulation instillation.

As used herein, eye drops, also known as ophthalmic drops or eye medications, are sterile solutions or suspensions administered directly into the eye for cosmetic, therapeutic, diagnostic, or prophylactic purposes. Eye drop formulations can vary widely depending on the specific outcome desired or condition being treated. Common types of eye drop formulations include sterile solutions, suspensions of solid particles dispersed in a liquid vehicle, emulsions i.e. biphasic formulations containing both water and oil phases stabilized with emulsifiers, gels having a semisolid consistency to provide prolonged contact time with the ocular surface and ophthalmic ointments which are semisolid preparations containing one or more active pharmaceutical ingredients (APIs) dispersed in a petrolatum base.

In some embodiments, the OCA2 modulating agent or any composition comprising the same may be administered via the scleral-conjunctival pathways or other pathways found between in any form of administration such as subconjunctival, intravitreal, intracameral, subretinal, systemic, etc.

In some other embodiments, the administration of the OCA2 modulating agent or any composition and pharmaceutical composition thereof may be via a depot injection. A depot injection may release the agent in a consistent way over a prolonged time period. Thus, a depot injection may reduce the frequency of dosing needed to obtain a desired effect, e.g., inhibition of OCA2, or brightening of the tissue. Depot injections may be intraocular injections, subcutaneous injections or intramuscular injections. In particular embodiments, the depot injection is an intraocular injection.

In some embodiments, the OCA2 modulating agent or any composition or specific pharmaceutical composition comprising the same in accordance with the present disclosure can be administered via topical administration. For topical administration, pharmaceutical compositions and formulations can include transdermal patches, ointments, lotions, creams, gels, drops, suppositories, sprays, liquids, and powders. It may be necessary or desirable to use conventional pharmaceutical carriers, e.g. aqueous, powdered, or oily bases, thickeners, and the like. Suitable topical formulations include those containing the OCA2 modulating agent featured in the invention combined with lipids, liposomes, fatty acids, esters of fatty acids, steroids, chelating agents, or surfactants. Suitable lipids and liposomes include neutral (e.g., dioleoylphosphatidyl DOPE ethanolamine, dimyristoylphosphatidyl choline DMPC, distearolyphosphatidyl choline) negative (e.g., dimyristoylphosphatidyl glycerol DMPG) and cationic (e.g., dioleoyltetramethylaminopropyl DOTAP and dioleoylphosphatidyl ethanolamine DOTMA) or ionizable.

In some embodiments, the OCA2 modulating agent or any composition or specific pharmaceutical composition comprising the same in accordance with the present disclosure may be formulated in a solution, e.g. a buffered saline solution such as PBS, or a gel for topical administration to the eye, such as, for example, in the form of eyedrops. In such embodiments, the formulations may include viscosity, tonicity and wetting agents, as well as preservatives, buffers and lubricants. These include but not limited to cationic emulsions and/or contain biopolymers such as, poly(lactide-co-glycolide), carbopol, hialuronic acid and polyacrylic acid.

In some further embodiments, the OCA2 modulating agent or any composition or specific pharmaceutical composition comprising the same in accordance with the present disclosure may be administered via a controlled drug delivery device such as a contact lens. In some embodiments, it does not significantly impair or interfere with the patient's vision. The contact lens consists of an optical pathway through which the wearer's line of sight passes. Further, the contact lens contains a substantially continuous drug-carrying zone, which contains at least one drug that will be released by the contact lens close to the eye.

In certain embodiments, the compositions or pharmaceutical compositions of the present disclosure may comprise an effective amount of the modulating agent of the invention. Still in some further embodiments, the method of the invention may refer to administration of an effective amount of the OCA2 modulating agent or any composition or specific pharmaceutical composition comprising the same.

The term “effective amount” relates to the amount of an active agent present in a composition/pharmaceutical composition, specifically the OCA2 modulating agent of the invention as described herein that is needed to provide a desired level of active agent in the bloodstream or at the site of action in a subject (e.g., the eye) to provide the desired effect i.e. physiological response when such composition is administered. The precise amount will depend upon numerous factors, e.g., the active agent, the activity of the composition, optimized stability in storage, safety profile, regulatory agency familiarity, the delivery device employed, the physical characteristics of the composition, intended patient use (i.e., the number of doses administered per day), patient considerations, and the like, and can readily be determined by one skilled in the art, based upon the information provided herein. An “effective amount” of the OCA2 modulating agent of the invention can be administered in one administration, or through multiple administrations of an amount that total an effective amount. It can be determined using standard clinical procedures for determining appropriate amounts and timing of administration. It is understood that the “effective amount” can be the result of empirical and/or individualized (case-by-case) determination on the part of the treating health care professional and/or individual. The active agent may be formulated for immediate activity or it may be formulated for sustained release.

In certain embodiments, the OCA2 modulating agent or any composition and pharmaceutical composition thereof is administered to a subject as a fixed dose. A “fixed dose” (e.g., a dose in mg) means that one dose of the agent is used for all subjects regardless of any specific subject-related factors, such as age/weight. In other embodiments, the agent of the invention is administered to a subject as a weight-based dose. A “weight-based dose” (e.g., a dose in mg/kg) is a dose of the RNA therapeutic agent that will change depending on the subject's weight.

In some embodiments, said pharmaceutical composition is for use in a method of modifying the eye color of a subject comprising administering to said subject an effective amount of at least one Oculocutaneous Albinism 2 (OCA2) protein modulating agent, or any vehicle, matrix, nano- or micro-particle thereof or any composition comprising the same.

In some further embodiments, said pharmaceutical composition is for use in a method of treating a disease/disorder in a subject in need thereof comprising administering to said subject an effective amount of at least one Oculocutaneous Albinism 2 (OCA2) protein modulating agent or any vehicle, matrix, nano- or micro-particle thereof or any composition comprising the same.

In yet another aspect, the present disclosure provides a nucleic acid molecule for modifying the eye color of a subject comprising at least one Oculocutaneous Albinism 2 (OCA2) protein modulating agent.

In some embodiments, said modulating agent is at least one gene editing agent adapted for modulating the expression and/or activity and/or stability of OCA2.

In some embodiments, said nucleic acid molecule comprises at least one of a single stranded DNA (ssDNA), a single stranded RNA (ssRNA), a double stranded DNA (dsDNA), a double stranded RNA (dsRNA), nucleic acid molecule having at least one modified nucleotide/s and any combinations thereof.

In some embodiments, said nucleic acid molecule comprises at least one of a small interfering RNA (siRNA), an antisense oligonucleotide (ASO), a guide RNA, a short hairpin RNA (shRNA), microRNA (miRNA), Peptide-Nucleic Acid (PNA) and locked nucleic acid (LNA). In some specific embodiments, said nucleic acid molecule comprises at least one siRNA molecule, wherein said siRNA molecule targets at least on sequence coding or controlling directly or indirectly the levels and/or activity and/or stability of OCA2.

In yet some alternative embodiments, said nucleic acid molecule comprises at least one ASO.

In some embodiments, said nucleic acid molecule targets at least one region in the OCA2 transcript.

In another embodiments, the nucleic acid molecule targets at least one region in the OCA2 gene.

In certain embodiments, the nucleic acid molecule targets at least one region in the OCA2 transcript that is susceptible to RNA-Induced Silencing Complex (RISC)-mediated cleavage.

In some embodiments, the nucleic acid molecule inhibits and/or reduces the expression and/or activity and/or stability of OCA2.

In some embodiments, said inhibition reduces the levels of melanin in the iris tissue of said subject.

In some specific embodiments, said inhibition reduces the average maturation of melanosomes. In some more specific embodiments, said inhibition reduces the average maturation of melanosomes in the iris stroma.

In some embodiments, the nucleic acid molecule reduces the expression and/or activity and/or stability of OCA2, said reduction results in lightening the eye color of said subject.

In some embodiments, the nucleic acid molecule targets the iris stroma and/or in specific embodiments, it targets the iris stromal melanocytes.

In some embodiments, the nucleic acid molecule targets the cells of at least one of the pigmented epithelium, the uvea, the choroid, the ciliary body, the Retinal Pigment Epithelium (RPE) and/or the Iris Pigment Epithelium (IPE).

In some embodiments, the nucleic acid molecule penetrates through the cornea and enter the anterior chamber of the eye.

In some embodiments, the nucleic acid molecule escapes destruction by the endo-lysosomal system.

In some embodiments, the nucleic acid molecule does not activate the immune system.

In some embodiments, said nucleic acid molecule is encapsulated within or coated with at least one nano-carrier.

In certain embodiments, the nucleic acid molecule is for use as a cosmetic composition.

In a further aspect, the present disclosure provides a method of treating a disease/disorder in a subject in need thereof comprising administering to said subject an effective amount of at least one Oculocutaneous Albinism 2 (OCA2) protein modulating agent or any vehicle, matrix, nano- or micro-particle thereof or any composition comprising the same.

In some embodiments, said disease or disorder is an OCA2 associated disorder. As used herein, an “OCA2 associated disorder” relates to any disorder/disease that is affected by the expression and/or activity of OCA2 in a subject. Thus, a OCA2 associated disorder may be any disorder which treatment/prophylaxis/care may benefit from modulation of OCA2, i.e. increasing or decreasing of the expression and/or activity of OCA2. An “OCA2 associated disorder” may also refers to a disorder which treatment/prophylaxis/care or associated side effects may benefit from modulation of OCA2. An example of a disorder which treatment/prophylaxis/care or associated side effects may benefit from modulation of OCA2 is glaucoma, specifically eye discoloration caused by glaucoma medication. Other examples of disorders which treatment/prophylaxis/care or associated side effects may benefit from modulation of OCA2 are pigmentary ocular disorder and Fuchs Heterochromic Iridocyclitis.

In some specific embodiments, said disease may be any one of hyperpigmentation ocular disorders; hyperpigmentation; side effects due to glaucoma medications; acquired heterochromia (e.g., due to Horner syndrome or Fuchs Heterochromic Iridocyclitis) and congenital heterochromia (e.g., due to genetic syndromes such as Waardenburg Syndrome); Bilateral Diffuse Uveal Melanocytic Proliferation (BDUMP); ocular nevi (e.g., choroidal nevus); Pigment Dispersion Syndrome; ocular melanoma; complexion-associated melanosis; primary acquired melanosis; Freckle; Lisch nodule; melanocytoma; ocular/oculodermal melanocytosis (e.g., Nevus of Ota); and benign ocular growths or any pigmentary ocular disorder, as well as pigmentary disorders presenting in the eye but are due to systemic conditions such as Addison's disease.

In some embodiments, said disease/disorder is an eye disorder. In some embodiments, said eye disorder is any one of heterochromia, Horner syndrome, hyperpigmentation or ocular glaucoma.

As used herein, the term “Pigmentary ocular disorder” relates to a group of eye conditions characterized by abnormal pigmentation within or around the eye. These disorders involve the deposition, migration, or distribution of pigmented substances in various ocular tissues, which can affect vision and eye health. Pigmentary ocular disorders can involve both the anterior segment (front portion) and posterior segment (back portion) of the eye. Examples of common pigmentary ocular disorders include but are not limited to Pigment Dispersion Syndrome (PDS), Pigmentary Glaucoma, Ocular Melanosis, Choroidal Nevus and Choroidal Melanoma.

Hyperpigmentation in ocular disorders refers to the darkening of tissues within or around the eye due to increased melanin production or accumulation. This condition can affect various parts of the eye, including the eyelids, conjunctiva, iris, and retina. Hyperpigmentation can be caused by a variety of factors, including inflammation, trauma, genetic predisposition, certain medications, and underlying systemic diseases. in ocular disorders, hyperpigmentation can manifest in different ways such as eyelid hyperpigmentation, conjunctival hyperpigmentation, iris hyperpigmentation and retinal hyperpigmentation.

Ocular glaucoma is a group of eye diseases characterized by damage to the optic nerve, often associated with elevated intraocular pressure (IOP). The optic nerve is responsible for transmitting visual information from the eye to the brain. When damage occurs to this nerve, it can result in vision loss and, if left untreated, eventual blindness. There are two main categories of glaucoma, open-angle glaucoma and angle-closure glaucoma.

Open-angle glaucoma is the most common form of glaucoma. In open-angle glaucoma, the drainage angle of the eye remains open, but the trabecular meshwork, which is responsible for draining the aqueous humor (fluid) from the eye, becomes less efficient over time. This leads to a gradual increase in intraocular pressure, which can damage the optic nerve.

In angle-closure glaucoma, the drainage angle of the eye becomes blocked or narrowed, preventing the aqueous humor from draining properly. This can lead to a sudden increase in intraocular pressure, which is known as an acute angle-closure attack. This type of glaucoma requires immediate medical attention as it can cause rapid vision loss if left untreated.

As mentioned further in the present disclosure, the OCA2 modulating agent of the invention may alleviate side effects (darkening of the eyes)—caused by medications used to treat glaucoma.

Horner syndrome, also known as Horner's syndrome or oculosympathetic palsy, is a rare condition characterized by a specific combination of symptoms resulting from damage to the sympathetic nervous system, typically affecting one side of the face and eye. The syndrome is named after Swiss ophthalmologist Johann Friedrich Horner, who first described it in 1869. The classic triad of symptoms associated with Horner syndrome includes ptosis, miosis and anhidrosis.

Heterochromia, as used herein, is a condition characterized by a difference in coloration of the iris, the colored part of the eye surrounding the pupil. It can manifest as one iris being a different color from the other (complete heterochromia) or as variations in color within a single iris (sectoral or partial heterochromia).

Fuchs heterochromic iridocyclitis (FHI) is a rare and chronic inflammatory eye condition that primarily affects the iris (the colored part of the eye) and the ciliary body (the structure behind the iris that produces aqueous humor). It is characterized by a distinctive combination of symptoms and findings, including iris heterochromia (a difference in color between the affected and unaffected eye), anterior chamber inflammation (iritis), and glaucoma. Key hall marks of Fuchs heterochromic iridocyclitis are heterochromia, anterior chamber inflammation (iritis) and glaucoma.

In some further embodiments, the hyperpigmentation disease is due to prostaglandin and/or prostaglandin analogues used against ocular glaucoma.

In some specific embodiments, said disease/disorder/condition is caused by glaucoma medication, specifically said disease/disorder/condition refers to eye discoloration caused by glaucoma medication.

In some embodiments, the effect is reversible and/or does not kill melanocytes and/or and does not adversely affect pigment production in other tissues of the eye, such as the retinal pigment epithelium.

In some embodiments, the composition to be administered in accordance with the method of treating of the present disclosure is as defined in the previous aspects described above.

In some embodiments, said at least one OCA2 modulating agent comprises at least one of nucleic acid molecule or any vector or host cell comprising the same.

Vectors, as used herein, refer to nucleic acid molecules with specific inserted sequences that can be introduced into a host cell, resulting in the creation of a transformed host cell. A vector may include nucleic acid sequences that permit it to replicate in a host cell, such as an origin of replication. A vector may also include one or more selectable marker genes and other genetic elements known in the art, including promoter elements that direct nucleic acid expression. Many vectors, e.g. plasmids, cosmids, minicircles, phage, viruses, etc., useful for transferring nucleic acids into target cells may be applicable in the present invention. The vectors comprising the nucleic acid(s) may be maintained episomally, e.g. as plasmids, minicircle DNAs, viruses such cytomegalovirus, adenovirus, etc., or they may be integrated into the target cell genome, through homologous recombination or random integration, e.g. retrovirus-derived vectors such as AAV, MMLV, HIV-1, ALV, etc.

Vectors may be provided directly to the subject cells. In other words, the cells are contacted with vectors comprising the nucleic acid molecule of the invention such that the vectors are taken up by the cells. Methods for contacting cells with nucleic acid vectors that are plasmids, such as electroporation, calcium chloride transfection, and lipofection, are well known in the art. DNA can be introduced as naked nucleic acid, as nucleic acid complexed with an agent such as a liposome or poloxamer, or can be delivered by viruses (e.g., adenovirus, AAV).

More specifically, in some embodiments, the vector may be a viral vector. In yet some particular embodiments, such viral vector may be any one of recombinant adeno associated vectors (rAAV), single stranded AAV (ssAAV), self-complementary rAAV (scAAV), Simian vacuolating virus 40 (SV40) vector, Adenovirus vector, helper-dependent Adenoviral vector, retroviral vector and lentiviral vector.

As indicated above, in some embodiments, viral vectors may be applicable in the present invention. The term “viral vector” refers to a replication competent or replication-deficient viral particle which are capable of transferring nucleic acid molecules into a host.

In some embodiments, the nucleic acid molecule of the invention may be comprised within an Adeno-associated virus (AAV). The term “adenovirus” is synonymous with the term “adenoviral vector”. AAV is a single-stranded DNA virus with a small (~20 nm) protein capsule that belongs to the family of parvoviridae, and specifically refers to viruses of the genus adenoviridiae. The term adenoviridiae refers collectively to animal adenoviruses of the genus mastadenovirus including but not limited to human, bovine, ovine, equine, canine, porcine, murine and simian adenovirus subgenera. In particular, human adenoviruses includes the A-F subgenera as well as the individual serotypes thereof the individual serotypes and A-F subgenera including but not limited to human adenovirus types 1, 2, 3, 4, 4a, 5, 6, 7, 8, 9, 10, 11 (AdllA and Ad IIP), 12, 13, 14, 15, 16, 17, 18, 19, 19a, 20, 21, 22, 23, 24, 25, 26, 27, 28, 29, 30, 31, 32, 33, 34, 34a, 35, 35p, 36, 37, 38, 39, 40, 41, 42, 43, 44, 45, 46, 47, 48, and 91.

In yet some further embodiments, HDAd vectors may be suitable for the methods of the invention. The Helper-Dependent Adenoviral (HDAd) vectors HDAds have innovative features including the complete absence of viral coding sequences and the ability to mediate high level transgene expression with negligible chronic toxicity. HDAds are constructed by removing all viral sequences from the adenoviral vector genome except the packaging sequence and inverted terminal repeats, thereby eliminating the issue of residual viral gene expression associated with early generation adenoviral vectors.

Still further, in some embodiments, SV40 may be used as a vector suitable for the present disclosure. SV40 vectors (SV40) are vectors originating from modifications brought to Simian virus-40 an icosahedral papovavirus. Recombinant SV40 vectors are good candidates for gene transfer, as they display some unique features: SV40 is a well-known virus, non-replicative vectors are easy-to-make, and can be produced in titers of 10 (12) IU/ml. They also efficiently transduce both resting and dividing cells, deliver persistent transgene expression to a wide range of cell types, and are non-immunogenic. Present disadvantages of rSV40 vectors for gene therapy are a small cloning capacity and the possible risks related to random integration of the viral genome into the host genome.

In certain embodiments, an appropriate vector that may be used by the invention may be a retroviral vector. A retroviral vector consists of proviral sequences that can accommodate the gene of interest, to allow incorporation of both into the target cells. The vector may also contain viral and cellular gene promoters, to enhance expression of the gene of interest in the target cells. Retroviral vectors stably integrate into the dividing target cell genome so that the introduced gene is passed on and expressed in all daughter cells. They contain a reverse transcriptase that allows integration into the host genome.

In yet some alternative embodiments, lentiviral vectors may be used in the present invention. Lentiviral vectors are derived from lentiviruses which are a subclass of Retroviruses. Commonly used retroviral vectors are “defective”, i.e. unable to produce viral proteins required for productive infection. Methods of introducing the retroviral vectors comprising the nucleic acid molecule of the invention into a target cell of interest are well known in the art.

In some alternative embodiments, the vector may be a non-viral vector. More specifically, such vector may be in some embodiments any one of plasmid, minicircle and linear DNA.

Nonviral vectors, in accordance with the invention, refer to all the physical and chemical systems except viral systems and generally include either chemical methods, such as cationic liposomes and polymers, or physical methods, such as gene gun, electroporation, particle bombardment, ultrasound utilization, and magnetofection.

For example, physical methods applied for in vitro and in vivo gene delivery are based on making transient penetration in cell membrane by mechanical, electrical, ultrasonic, hydrodynamic, or laser-based energy so that DNA entrance into the targeted cells is facilitated.

In more specific embodiments, the vector may be a naked DNA vector. More specifically, such vector may be for example, a plasmid, minicircle or linear DNA.

It should be appreciated that all DNA vectors disclosed herein, may be also applicable in the all the different aspects of the invention.

Still further, it must be appreciated that the invention further provides any vectors or vehicles that comprise any of the nucleic acid molecules disclosed by the invention, as well as any host cell expressing the nucleic acid molecules disclosed by the invention.

In some embodiments, said at least one nucleic acid molecule or any vector or host cell comprising the same is as denoted in the previous aspect of the present disclosure detailed above.

The present disclosure also relates to any vector comprising the nucleic acid molecules as defined above and to any host cell comprising said vector.

In some embodiments, said at least one OCA2 modulating agent is administered via ocular administration.

In some embodiments, said at least one OCA2 modulating agent is administered via topical administration and/or in some specific embodiments into the eye of said subject.

In certain embodiments, said at least one OCA2 modulating agent is comprised in an eye drop formulation.

In a yet additional aspect, the present disclosure provides at least one Oculocutaneous Albinism 2 (OCA2) protein modulating agent or any vehicle, matrix, nano- or micro-particle thereof or any composition comprising the same for use in a method of treating a disease/disorder in a subject in need thereof comprising administering to said subject an effective amount of said at least one OCA2 protein modulating agent.

It should be noted that all the previously described embodiments with respect to the method of treating a disease/disorder aspect defined above are also applicable to this aspect of the present disclosure relating to the OCA2 modulating agent for use in accordance with the present disclosure.

It is to be understood that the terms “treat”, “treating”, “treatment” or forms thereof, as used herein, mean preventing, ameliorating or delaying the onset of one or more clinical indications of disease activity in a subject having a pathologic disorder. Treatment refers to therapeutic treatment. Those in need of treatment are subjects suffering from a pathologic disorder. Specifically, providing a “preventive treatment” (to prevent) or a “prophylactic treatment” is acting in a protective manner, to defend against or prevent something, especially a condition or disease. The term “treatment or prevention” as used herein, refers to the complete range of therapeutically positive effects of administrating to a subject including inhibition, reduction of, alleviation of, and relief from, a condition, illness and signs and symptoms thereof or undesired side effects and cosmetic hurdle that the patients would like to change. More specifically, treatment or prevention of relapse or recurrence of the disease, includes the prevention or postponement of development of the disease, prevention or postponement of development of symptoms and/or a reduction in the severity of such symptoms that will or are expected to develop. These further include ameliorating existing symptoms, preventing-additional symptoms and ameliorating or preventing the underlying causes of symptoms.

As indicated above, the methods and compositions provided by the present invention may be used for the treatment of a “pathological disorder” which refers to a condition, in which there is a disturbance of normal functioning, any abnormal condition of the body or mind that causes discomfort, dysfunction, or distress to the person affected or those in contact with that person. It should be noted that the terms “disease”, “disorder”, “condition” and “illness”, are equally used herein.

It should be appreciated that any of the methods and compositions described by the invention may be applicable for treating and/or ameliorating any of the disorders disclosed herein or any condition associated therewith. It is understood that the interchangeably used terms “associated”, “linked” and “related”, when referring to pathologies herein, mean diseases, disorders, conditions, or any pathologies which at least one of: share causalities, co-exist at a higher than coincidental frequency, or where at least one disease, disorder condition or pathology causes the second disease, disorder, condition or pathology. More specifically, as used herein, “disease”, “disorder”, “condition”, “pathology” and the like, as they relate to a subject's health or cosmetic preference e.g., unwanted ocular pigmented lesions, acquired or congenital heterochromia, are used interchangeably and have meanings ascribed to each and all of such terms.

The present invention relates to the treatment of subjects or patients, in need thereof. By “patient”, “subject” or “subject in need” it is meant any organism who may be affected by the above-mentioned conditions, and to whom the therapeutic and prophylactic methods herein described are desired, including humans, domestic and non-domestic mammals such as canine and feline subjects, bovine, simian, equine and rodents, specifically murine subjects. More specifically, the methods of the invention are intended for mammals. By “mammalian subject” is meant any mammal for which the proposed therapy is desired, including human, livestock, equine, canine, and feline subjects, most specifically humans. It is therefore intended that the term “subject” refers to any animal, human or non-human, preferably vertebrates, and more preferably mammals. It is possible for a subject to include a transgenic organism. It is most preferred that the subject is human.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range. In some embodiments, the term “about” refers to ±10%.

The indefinite articles “a” and “an,” as used herein in the specification and in the claims, unless clearly indicated to the contrary, should be understood to mean “at least one.” It must be noted that, as used in this specification and the appended claims, the singular forms “a”, “an” and “the” include plural referents unless the content clearly dictates otherwise.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

Throughout this specification and the Examples and claims which follow, all transitional phrases such as “comprising,” “including,” “carrying,” “having,” “containing,” “involving,” “holding,” “composed of,” and the like are to be understood to be open-ended, i.e., to mean including but not limited to. Specifically, it should understood to imply the inclusion of a stated integer or step or group of integers or steps but not the exclusion of any other integer or step or group of integers or steps. Only the transitional phrases “consisting of” and “consisting essentially of” shall be closed or semi-closed transitional phrases, respectively, as set forth in the United States Patent Office Manual of Patent Examining Procedures. More specifically, the terms “comprises”, “comprising”, “includes”, “including”, “having” and their conjugates mean “including but not limited to”. The term “consisting of means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

It should be noted that various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated herein above and as claimed in the claims section below find experimental support in the following examples. Disclosed and described, it is to be understood that this invention is not limited to the particular examples, methods steps, and compositions disclosed herein as such methods steps and compositions may vary somewhat. It is also to be understood that the terminology used herein is used for the purpose of describing particular embodiments only and not intended to be limiting since the scope of the present invention will be limited only by the appended claims and equivalents thereof.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Without further elaboration, it is believed that one skilled in the art can, using the preceding description, utilize the present invention to its fullest extent. The following preferred specific embodiments are, therefore, to be construed as merely illustrative, and not limitative of the claimed invention in any way.

### Experimental Procedures

### Melanin Absorbance Quantification Reagents

The following materials and reagents were used: Melanin (Sigma, Cat #M0418), Ammonium Hydroxide (Sigma, Cat #09859), Dulbecco Modified Eagle Medium (DMEM) (Biowest, Cat #L0102-500), Fetal Bovine Serum (FBS) (Gibco, Cat #10270-106), Penicillin/Streptomycin (Pen/Strep) (Gibco, Cat #15140-122), Standard 96-well plates (Greiner, Cat #655101), UV-star 96-well plates (Greiner, Cat #675801), B16-F10 mouse melanoma cells (ATCC, Cat #CRL-6475), Trypsin-EDTA at 0.25% (Gibco, Cat #25200-072), Phosphate-Buffered Saline (PBS) (Sartorius, Cat #02-023-1A), Trypan Blue (Sigma, Cat #T8154), T75 flasks (Greiner, Cat #658170), Nunclon Delta 6-well plates (Thermo, Cat #140675), ViewPlate white 96-well plate, clear bottom (Perkin Elmer, Cat #6005181) and CellTiter-Glo (Promega).

### Cell Titer Glo Assay

CellTiter-Glo® Buffer was thawed and equilibrated to room temperature before use, and stored at 4° C. for up to 48 hours. The lyophilized CellTiter-Glo® Substrate was brought to room temperature. 10 ml of CellTiter-Glo® Buffer was added to the substrate to form the CellTiter-Glo®Reagent, which was stored at −20° C. when not in use. After 72 hours of incubation, the plate was brought to room temperature for about 30 minutes. 100 μl/well of CellTiter-Glo® Reagent was added. The plate was shaken for 2 minutes on an orbital shaker for cell lysis. The plate was incubated at room temperature for 10 minutes to stabilize the luminescent signal. Luminescence was recorded using a Clariostar BMG plate reader with an integration time of 1 second per well.

siRNA Transfection & qPCR mRNA (OCA2) in B16-F10 Melanoma Cells

B16-F10 mouse melanoma cells (ATCC CRL-6475) were cultured in DMEM supplemented with FBS and Pen/Strep, and passaged using 0.25% Trypsin-EDTA. For the transfection process, various test items were employed, including three different OCA2 siRNAs and control siRNAs (scramble siRNA and GAPDH siRNA (Thermo-Fisher). Transfection controls included untransfected cells and cells treated with Block-iT.

For 6-well plates, seeding concentrations of 250,000 and 300,000 cells/well were used for expression experiments, reaching 60-80% confluence 24 hour later. Medium was changed every 2-3 days. For 96-well plates, seeding concentrations of 7,500 and 10,000 cells/well were used.

### Cell Transfection Using OptiMEM-Lipofectamine

The transfection protocol involved preparing OptiMEM-lipofectamine mix in 1.5 ml Eppendorf tubes, followed by the preparation of master mixes of mRNA in a separate Eppendorf tube. The diluted Lipofectamine RNAiMax Reagent was then added to each tube of diluted RNA in a 1:1 ratio. This mixture was incubated for 5 minutes at room temperature before being added to the cells (250 μl/well). The cells were then incubated at 37° C. in a 5% CO₂ environment. Post-transfection, cells were collected for FACS analysis and microscope observation at 24 hours, and for RNA extraction at 48 hours.

### Iris Samples Transfection Using OptiMEM-Lipofectamine

The siRNA and Lipofectamine were preincubated with Opti-MEM to achieve final concentrations of 50 nM for siRNA and 5 μl/ml for Lipofectamine. Following preincubation, 100 μl of the siRNA-Lipofectamine solution was added to wells containing irises in 500 μl of culture media. Transfection was carried out in a final volume of 600 μl.

Transfection Control with BLOCK-iT—Microscopy & FACS

Lipofectamine RNAiMax (Thermo, Cat #13778-030) and Block-iT fluorescent oligo (Thermo, Cat #2013) were diluted separately in OptiMEM (Gibco, Cat #31985-047). The two solutions were then mixed in a 1:1 ratio and incubated for 5 minutes at room temperature. The RNA-lipid complex was added to the cells in a volume of 250 μl per well for 6-well plates and 10 μl per well for 96-well plates. Post-transfection, cells were incubated at 37° C. and assessed 3-6 hours later using a fluorescent microscope and 24-48 hours later using both a fluorescent microscope and Flow Cytometry (FACS).

Viability Test after Transfection

Cell viability was assessed using the Cell Titer Glo assay (Promega) 24 hours post-transfection.

FACS analysis was performed to assess transfection efficiency. Cells were washed with PBS, trypsinized, resuspended in growth medium, and then transferred to a 96-well U-bottom plate for analysis.

### RNA Extraction and PCR

B16-F10 cell pellets were resuspended in TRI Reagent® according to cell count (100 μl for ≤10\hat{ }5 cells, 300 μl for ≤10\hat{ }6 cells, and 600 μl for ≤5*10\hat{ }6 cells). All samples were standardized to 300 μl with TRI Reagent®. RNA Purification: lysed samples were mixed with an equal volume of 95-100% ethanol and transfer to Zymo-Spin™ IICR Columns for centrifugation. DNase I treatment was performed on the column, followed by washing with RNA Wash Buffer and Direct-zol™ RNA PreWash. RNA was eluted with DNase/RNase-Free Water and concentration and purity were measured using Nanodrop. Reverse Transcription of RNA to cDNA was performed as previously described.

### Preparation and Analysis of Murine Ex Vivo Iris Explants

Study Design and Ethical Considerations Ethical approval was granted by the “Committee for Ethical Conduct in the Care and Use of Laboratory Animals”. The study adheres to the Animal Welfare Law (Animal Studies)—1994 (State of Israel), the Guide for the Care and Use of Laboratory Animals by the Institute of Laboratory Animal Research (ILAR), and the Guidelines of the National Institute of Health (NIH).

The experiment utilized C57BL/6JOlaHsd & DBA2/J mice ex-vivo iris models. Supplied by Envigo CRS (Israel) LTD. Specific pathogen-free (SPF). Approximate Age: 1-2 months.

### Iris Samples Micro-Dissections:

Mice were anesthetized using a Ketamine/Xylazine mix and subsequently euthanized. Eye dissection employed a stereoscopic dissecting microscope with a cold light source to minimize glare and reflections.

Dissection of irises was conducted as following: Immobilization of the enucleated eyeball; Making an incision at the corneal limbus for entry; Dividing the eye into anterior and posterior segments and separating the circumference. Identification and dissection of the neural retina from the posterior eye cup; Incising behind the limbus using a sharp razor blade; Continuing the incision around the globe with curved iris scissors; Gently removing the lens using fine forceps; Creating pie-shaped wedges from the anterior segment; Removing excess sclera, choroid, and retina posterior to the ciliary body; Gripping the central cornea with fine forceps and a Tooke's knife; Breaking adhesions between the iris-ciliary body and cornea-sclera; Transferring isolated iris-ciliary body pieces into 24 well plates containing media.

### Iris Samples Culture & Viability Evaluation

Media and Supplements: TC DMEM F12 (Gibco, cat #11320074), Pen/Strep (Gibco, cat #15140-122), Sodium pyruvate (Sartorius, cat #03-042-1B), NEAA (Gibco, cat #11140-035), FBS (Gibco, cat #10270-106), B27 (Gibco, cat #17507-044).
Medium A: DMEM F12 with 10% FBS, 1×NEAA, 1× Sodium pyruvate, 1× Pen/Strep
Medium B: DMEM F12 with 20% FBS, 1×NEAA, 1× Sodium pyruvate, 1× Pen/Strep
Medium C: DMEM F12 with 1× B27, 1×NEAA, 1× Sodium pyruvate, 1× Pen/Strep

For Viability Testing: PrestoBlue™ Cell Viability Reagent/25 ml (Rhenium, cat #A13261) was used.

Washing Protocol: each iris was transferred through three wells containing sterile media for decontamination.

One iris was then immersed in 70% EtOH for 10 minutes, then washed to serve as no-viability iris.

Viability Quantification: Presto Blue reagent was warmed to room temperature. 35 μl of Presto Blue was added to irises in culture medium; 25 μl to culture mediums. Incubation for 2.5 hours at 37° C. The 12 murine irises were incubated for 2.5 hours in 100 μl media containing 10% Presto blue. Following incubation 50 μl of each sample was transferred to a black 96-well plate. Absorbance and Fluorescence Intensity were measured.

### SOX10 Knockdown in Iris Samples

Mice Irises harvested as described herein were utilized in this study. The irises were maintained in Media A. For the transfection process, various test items were employed, including SOX10 siRNA with its nucleotide strands sequences:

Sense:
5′-AGCCAGUAUAUACGACUCUAUCCCG-3′
as denoted by SEQ ID NO: 76,
and
Antisense:
3′-CGUCGGUCAUAUAUGCUGAGAUAGGGC-5′
as denoted by SEQ ID NO: 77.

Irises treated with scrambled siRNA from Thermo-Fisher (catalogue-4390843) served as negative controls.

The transfection protocol involved preparing OptiMEM-lipofectamine mix in 1.5 ml Eppendorf tubes, followed by the preparation of master mixes of mRNA in a separate Eppendorf tube. The diluted Lipofectamine RNAiMax Reagent was then added to each tube of diluted RNA in a 1:1 ratio. This mixture was incubated for 5 minutes at room temperature before being added to the irises (100 μl/well). The irises were then incubated at 37° C. in a 5% CO₂ environment. 24 hours post-transfection, media was replaced. At 48 hours post transfection the irises were harvested for RNA extraction.

RNA Extraction from Iris Samples

The DirectZol RNA Miniprep Kit (Zymo Research, cat #R2053) with included Tri-reagent, Absolute Ethanol (Gadot), Maxima First Strand cDNA Synthesis Kit (Thermo Fisher), Ultrapure Water (Thermo Fisher), Taqman Fast Advanced Master Mix (Thermo Fisher), and PrimeTime Gene Expression Master Mix (IDT) were among the reagents used.

For Transfection experiment eight iris tissues were harvested from DBA mice. The experimental group was treated with Sox10-siRNA, and the control group received scrambled siRNA. SOX10 expression was assessed 48 hours post transfection. In addition to the 8 transfected irises, several other irises were harvested and immediately processed for RNA extraction and were evaluated for their SOX10, GAPDH and OCA2 expression as well, without undergoing transfection, for the optimization of RNA extraction process.

During homogenization and RNA extraction, iris samples were treated with TriReagent and stainless-steel beads, using a homogenizer set at speed 10 for three cycles of 1 minute each. This procedure facilitated the subsequent RNA extraction using a column-based kit, following the manufacturer's guidelines closely. The purity and concentration of extracted RNA were assessed using a NanoDrop spectrophotometer. The extraction process was performed with pooled iris samples to enhance RNA quantity.

Targeted depigmentation of iris stromal melanocytes in mammals.

### Ethical Committee Approval

The study was initiated following approval from the Committee for Ethical Conduct in the Care and Use of Laboratory Animals, ensuring compliance with pertinent regulations.

### Animal Welfare Compliance

The research adhered to:
The Animal Welfare Law (Animal Studies) of 1994 (State of Israel),
The ILAR's Guide for the Care and Use of Laboratory Animals,

### NIH Guidelines: Supply and Documentation

The Sponsor provided the test item, storage solutions, and special materials, along with comprehensive documentation covering safety instructions for sample handling, storage, and item identification.

### Storage Solutions and Test Item Materials

Storage solutions and test items were meticulously catalogued, featuring products such as BLOCK-iT™ Alexa Fluor® Red Fluorescent Oligo, Invivofectamine® 3.0 Starter Kit, and Lipofectamine® RNAiMAX Reagent OCA2-siRNA provided by IDT and Thermo-Fisher, among others, supplied by Invitrogen and Gibco.

### Test Items Preparation/Formulation.

Invivofectamine® 3.0-Block-iT Complex Preparation: The preparation involves mixing Block-iT™ Alexa Fluor® with Opti-MEM® Medium, followed by complexation with Invivofectamine® 3.0 Reagent. The mixture is incubated, diluted, and stored appropriately before in vivo delivery.
Lipofectamine-Block-iT Complex Preparation: This process begins with the preparation of a Block-iT solution, followed by dilution of Lipofectamine® RNAiMAX reagent, and subsequent mixing and incubation to form the Lipofectamine® RNAiMAX-Block-iT complex for delivery.
Lipofectamine-OCA2-siRNA Complex Preparation: Diluted Lipofectamine RNAiMAX reagent is added to each tube of Diluted RNA (1:1 ratio)·

### Group Assignment and Administration

Animals are assigned to groups based on weight to ensure homogeneous treatment groups. The administration of test items is executed via Intravitreous (IVT) or Anterior Chamber (AC) injections, with anesthesia protocols detailed for each procedure. Groupings for IVT and AC injections are delineated, and specific equipment is used for the microsurgery procedures.

The study methodically organizes subjects into specific groups, each distinguished by the route of administration and the test items used, along with varying dosages to assess their effects on dissected iris specimens. Subjects are designated to receive either Invivofectamine® 3.0-Block-iT or Lipofectamine® RNAiMAX via intravitreal (IVT) or anterior chamber (AC) injections, spanning a range of dosages. Each experimental group includes two subjects, with the exception of the last two control groups, which are administered a placeholder treatment (TBD) and consisted of only one subject each. This setup facilitates a thorough examination of the impacts under varying conditions, significantly enriching the study outcomes.

### Dissection of Irises:

The final phase of the study, which occurs 24 hours after the administration of the test item, involves anesthetizing the animals with a Ketamine/Xylazine mix and subsequently euthanizing them. The dissection of the eyes is performed using a stereoscopic dissecting microscope, which is equipped with a cold light source to minimize glare and reflections from the metal dissection instruments. The detailed steps of the dissection process, as outlined in the previous description of the ex-vivo example, are meticulously followed to ensure precise examination and analysis of the iris specimens.

### Intravitreal Injection Procedure:

The procedure begins with creating a micropuncture on the conjunctiva of a mouse eye using a 31-gauge needle, positioned about 1.0 mm from the limbal region into the lateral sclera at the pars plana level. This initial step ensures a slight vitreous protrusion to lower intraocular pressure before the IVT injection. To avoid causing traumatic cataracts, the microneedle is inserted at a 45° angle into the vitreous body. The test item/vehicle is then slowly injected at a controlled rate using an intraocular injecting Hamilton syringe (GA33-34), achieving successful unilateral IVT injection. Following the injection, the needle is carefully withdrawn from the site after a short pause.

### Anterior Chamber (Intracameral) Injection Procedure:

Similarly, a micropuncture is made on the cornea using a 31-gauge needle, located about 1.0 mm from the limbal region into the anterior chamber, to initiate the AC injection. The microneedle is carefully inserted into the anterior chamber to prevent traumatic cataracts, followed by a slow injection of the test item/vehicle using an intraocular injecting Hamilton syringe (GA33-34) for successful unilateral AC injection. The needle is then slowly and cautiously removed from the injection site.

### Light Microscopy:

Iris samples or whole eyes are fixed in buffered formalin the tissue specimens are processed for paraffin embedding. Standard five micrometer sections are prepared and mounted onto glass slides. Parallel sections are processed for hematoxylin-eosin (HE) staining for the histopathological evaluation. The stained slides are studied microscopically and evaluated using commercially available Fontana Mason staining kit.

### Transmission Electron Microscopy (TEM) Preparation:

Irises and retinas are washed in 1 TBS and placed in 10%, 20%, and 30% gradient sucrose solution prepared in 1 pin before cryo embedding. Afterwards, tissues are embedded in cryo embedding solution, frozen in liquid nitrogen, and kept at −20° Celsius until further processing. Using the Leica cryotome CM1860, 10 mm thick sections are cut.

### Pre-TEM Embedding:

After 48 h of incubation, the transfected and control cells are harvested, and washed three times with 0.1 M phosphate buffer. These cells are primarily fixed by fixing solution containing 4% paraformaldehyde, 1% gluteraldehyde in 0.1 M Phosphate buffer for 4 h followed by treatment of 1% osmium tertroxide at 4 C for 1 h, for secondary fixation. After that dehydration is performed, using an ascending concentration of ethyl alcohol i.e., 10 min each in 50, 70, 80, and 90%, twice in 95% for 15 min, and finally 1 h in 100%. After this step, the sample is treated with propylene oxide for 15 min. Embed 812, a commercial quick-setting epoxy glue containing epoxy resin (mixtures A and B) and 1.4% DMP-30, is used to embed samples Mixture A contained Embed 812 (EMS) and dodecyl succinic anhydride, while Mixture B had Embed 812 (EMS) and nadic methyl anhydride. Hexylene glycol (HG) and resin (R) are combined in a 1:2 ratio, added to the dish, and mixed on the rotor for 2 hours. Next the resin and hexylene glycol are combined at a 2:1 ratio for 2 hours and the resin and hexylene glycol are replaced with 100% resin and mixed for 6 hours at 60 C for 3 days. Then ultrathin sections of the sample are obtained with an ultramicrotome (MTX ULTRAMICROTOME). The specimen is treated with Uranyl acetate and lead citrate for 30 and 15 min, respectively.

### Illumination and Imaging of Iris Samples

All samples (of both treatment group and control group) were subjected to a 9W 6500K LED light source, positioned 8.5 cm away, a distance previously validated as non-detrimental to incubated iris samples in an earlier study. This proximity was shown to neither alter the temperature surrounding the samples and the constant light exposure protocol was based on prior evidence indicating its safety and efficacy in maintaining the integrity and health of the iris samples under study. The exposure to this specific lighting was continuous throughout the experimental period, with brief intermissions for essential procedures such as photography, viability assessments, and the administration of transfection materials.

Imaging was performed at baseline and at day 14 of the experiment (3 days after the 4th transfection and immediately before fixating and processing for histological evaluation) using a Panasonic Lumix G9 camera equipped with a Laowa 25 mm f/2.8 Ultra Macro 2.5-5× lens. Both backlighting and ambient lighting conditions were rigorously controlled for their intensity and for their Kelvin (both at 6500K, mimicking daylight hue). Photographs were captured at f/16, ISO 200, with a shutter speed of 4″. All other photographic parameters were standardized, including room ceiling light, camera and iris positioning, vapor levels on the plate seal, and proximity to other light sources.

### Example 1

### Targeting of the OCA2 Gene

Targeting the expression of the OCA2 gene enables to attain the most aesthetically natural results, comparable to those who are born with blue eyes since the reduction in OCA2 protein levels and the downstream consequences in iridial melanocytes and iris tissue are natural in people who are born with bright eyes.

The advantage of this approach is heightened when compared to the reduction of OCA1 expression and its downstream consequences. OCA1 gene inhibition results in a reduction of the tyrosinase enzyme and reduced pigment in the retinal pigment epithelium (RPE) and iris pigmented epithelium (IPE). This may result in vision loss and other negative consequences. As opposed to the majority of blue-eyed individuals who inherit mutations that decrease OCA2 expression and who are generally healthy and of good vision, individuals with brightly colored eyes due to decreased tyrosinase activity and OCA1 gene expression suffer from health issues (such as seen in albinos with red iris and vision loss).

In this method, large molecules are delivered for example in eye drops that penetrate through the cornea and enter the anterior chamber of the eye, penetrate the iris stroma melanocytes efficiently, and escape destruction by the endo-lysosomal system without harming other cells or activating the immune system in the eye. In addition, a biologically targeted treatment is presented to reduce pigmentation in human and animal iridial melanocytes both in vivo and in vitro. Additionally, the method comprises siRNAs and/or antisense oligonucleotides that can be delivered as eye drops and specifically targets pigment production within the iris stroma. In addition, the treatment does not affect other eye functions nor trigger the immune system. It remains in the anterior chamber of the eye for long periods of time without being degraded. Moreover, since the silencing effect of RNA therapeutic agents is temporary, stopping the treatment results in gradual repigmentation. The result is a reversible eye color change, that does not kill melanocytes, and does not adversely affect pigment production in other tissues of the eye, such as the vital retinal pigment epithelium.

In IPE and RPE cells, modulation of OCA2 does not lead to the complete arrest of melanosome maturation as it does in stromal melanocytes. Therefore, OCA2 silencing has little or no impact on the level of pigmentation in these cells. In contrast, OCA1 silencing of tyrosinase inhibition has a substantial impact on pigmentation in these cells. First, histological studies using light microscopy and electron microscopy indicate that the pigmentation levels within the IPE are not significantly different between blue-eyed individuals (whose cells are known to express less OCA2) and brown-eyed individuals. However, there is a marked difference in the amount of pigmentation within the iris stroma. The majority of people with blue eyes also carry one of a few mutations all of which result in a reduction of OCA2 expression. There is, however, evidence that people with blue eyes have normal pigmentation in the iris pigmented epithelium (IPE). A properly pigmented IPE, as observed in individuals with both dark and bright colored eyes, but not in albinos due to the OCA1 mutation, reflects light back to an external observer without displaying the red coloration caused by blood vessels in the ciliary body. If it is absent, a pink-red hue is visible.

Targeted therapy interferes selectively with specific molecular pathways or genetic mutations that play a role in disease development and progression. These therapies have higher efficacy and safety potential than traditional non-specific therapies. The safety profile of targeted therapies is another advantage. Compared to traditional non-targeted therapies, targeted therapies have fewer side effects, which leads to improved quality of life for patients. This method involves a targeted therapy that targets Oculocutaneous Albinism 2 (OCA2), the level of which determines the amount of pigment produced within iridial melanocytes, which determines the color of the eyes. As a result of an SNP in an enhancer upstream of the OCA2 gene that regulates its expression, light-eyed and dark-eyed individuals display distinct phenotypic differences. When OCA2 expression in stromal melanocytes is low, melanosomes do not mature and pigmentation remains low. This doesn't seem to occur in pigmented epithelium cells of the eye (such as IPE and RPE) with low OCA2 expression. Thus, due to a few SNPs that result in the same phenotype, the vast majority of individuals with light eyes have decreased expression of the OCA2 gene. In contrast, people with dark eyes generally have adequate expression of the OCA2 gene. Based on this method, a targeted therapy mimics the above-mentioned SNPs closely to decrease levels of OCA2, bestow dysfunctional immature melanosomes into the iridial melanocytes of those born with normal levels of OCA2, reducing their pigmentation and altering their eye color.

### Example 2

### Endosomal Escape Modifications and Composition Delivery

Since the discovery of siRNA in the 2000s, the scientific community has been very excited about the potential for developing RNA silencing-based therapies. In an effort to develop such therapies, many pharmaceutical companies have entered the field. The majority of these attempts failed due to the inability of large molecules taken up by cells via endocytosis in the endosomes to escape degradation and reach the cytosol. This is where the target mRNA molecules are located. Over the past two decades, extensive research has been conducted on the endosome system, as well as effective techniques for escaping endosomes. In recent years, siRNAs and antisense oligonucleotides with the ability to reach the cytosol and effectively silence the target RNA have been commercialized. For example, by coating molecules with lipid nanoparticles (LNP), they are then able to escape endosomes at a greater rate.

It is possible to deliver RNA therapeutic agents into cells through the process of endocytosis, in which large molecules from the extracellular environment are internalized into the intracellular environment. In some pathways of endocytosis, a siRNA molecule binds to a specific cell surface receptor, with or without the help of a conjugate (such as GalNac, cholesterol, vitamin E, etc.). An endosome is formed when the cell membrane invaginates around the siRNA molecules. The endosome is then transported to the lysosome, where it is acidified and enzymatically degraded. However, siRNA molecules cannot silence gene expression inside endosomes as RNA interference occurs only within the cytoplasm.

This limitation can be overcome by making siRNA molecules more resistant to degradation or more likely to penetrate the endosome lipid bilayer. Specific delivery systems or chemical modifications to siRNA molecules are used to achieve this result. It is possible to increase the ability of these molecules to escape the endosome by encapsulating them in LNPs. For example, The endosomal escape efficiency of LNPs containing B-sitosterol has been demonstrated to be tenfold greater than that of LNPs without B-sitosterol (Herrera M, et L. (2021) Biomater Sci. 9 (12): 4289-300).

Thus, in order to brighten the color of the eyes, eye drops are produced whose active ingredient can reach the iris in a suitable concentration, enter the melanocytes, escape endosomal-lysosomal degradation, and silence the expression of OCA2.

In addition to non-viral cationic delivery systems, viral vectors such as adeno-associated viruses (AAV) and lentiviruses represent another class of effective delivery vehicles for RNA-based therapeutic agents. These viral vectors capitalize on the natural ability of viruses to enter cells, thereby offering a highly efficient method for the introduction of RNA molecules into target cells. The use of AAV or lentiviral vectors enables the stable expression of RNA agents, potentially overcoming challenges related to the degradation and transient expression associated with some non-viral delivery methods. The engineering and application of such viral delivery systems for therapeutic purposes are well-documented and within the expertise of those skilled in the art, providing a robust platform for the delivery of RNA-based therapies to a wide range of cell types, including those that are difficult to transfect using traditional methods.

### Example 3

### Composition Delivery and Pharmacokinetics

In order to topically deliver a composition through the cornea and into the anterior chamber, two main methods are available: eye drops and/or direct intraocular injections such as intracameral injections. Most patients prefer using eye drops, which can be administered at home without the assistance of a healthcare professional. Alternatively, injections increase bioavailability by bypassing the corneal barrier. However, patients perceive them to be deterrent, intimidating and painful. The preferred solution is to use eye drops that are designed to reach the iris at a sufficient concentration.

Having overcome the corneal barrier or the scleral-conjunctival barriers, the molecule is able to enter the melanocytes by endocytosis, escape the endosomal-lysosomal system, and finally silence OCA2. Compositions administered via topical eye drops, with their active ingredients reaching the anterior chamber have favorable biological distribution in the iris i.e. no systemic distribution, and a minimal distribution to the posterior chamber of the eye in animal models. The pharmacokinetics of these compositions between the ocular compartments provide a double advantage to local ocular drugs: first, they remain at a high concentration in the target area for a longer period of time, increasing their effectiveness and second they are not distributed to other organs, thereby reducing the drug burden and the likelihood of side effects significantly.

As an isolated organ, the eye is relatively protected from systemic dissemination of drugs, increasing their effectiveness (less degradation and more retention in the target area) and reducing their systemic and off-target effects. Compositions delivered topically to the eye surface can pass through the scleral conjunctival pathway (Leclercq B, Mejlachowicz D, Behar-Cohen F. Ocular Barriers and Their Influence on Gene Therapy Products Delivery. Pharmaceutics. 2022 May 6; 14 (5): 998). Alternatively, the composition could be delivered through the corneal epithelium in one of two primary ways: either by passing through the cells (transcellularly), which is the tendency of lipophilic molecules, or by passing around the cells (paracellularly), which is the tendency of hydrophilic molecules.

A major challenge of developing gene silencing therapies is the fact that the drugs are generally large and charged. This results in challenging pharmacokinetic properties for drug delivery. In addition, the drug must overcome a variety of barriers in order to be effective. These barriers include the anatomical barriers of the tear film and the cornea or the scleral-conjunctival pores, as well as entering the target cells, the stromal melanocytes, by endocytosis, and escaping degradation by the endosomal-lysosomal system.

In comparison with other organs, the eye has the advantage of being immune-privileged, which means that the immune system is relatively insensitive to foreign materials within the globe as compared to other organs. Furthermore, since the therapy is administered locally, there is no systemic uptake and no off-target effects are expected. These factors allow for safe and efficient local treatment of the eye.

### Example 4

Eye Drop Medication with RNA Interference (RNAi) Against OCA2

An eye drop medication is produced that induces the lightening of the iris through the targeted silencing of the OCA2 gene, effectively inhibiting melanin production within the melanocytes of the iris stroma. This therapeutic approach sets off a gradual depigmentation process by leveraging the natural photobleaching of existing melanin, hindered by the absence of new melanin synthesis to maintain pigment equilibrium. The treatment enables a progression through the natural spectrum of human eye colors, from dark brown to various shades of teal or gray, depending on the individual's response and treatment duration. Patients engaging in this regimen have the autonomy to adjust the treatment intensity to their preference, allowing for the halting of the lightening process at any desired point along the color spectrum, without necessitating a complete pigment absence. This customization is achieved not by ceasing treatment entirely but by extending the intervals between doses or transitioning to a maintenance dosage, thus preserving the attained eye color.

The aesthetic result of this treatment replicates the appearance of naturally lighter eyes, providing an alteration in iris coloration that is indistinguishable from individuals who were naturally born with light eyes. Should the treatment be adjusted to a maintenance regimen or the interval between doses increased, the lighter eye color is maintained. Conversely, if the treatment is significantly reduced or halted, melanocytes in the iris regain their ability to synthesize melanin, initiating a gradual return to the iris original hue. This repigmentation process is akin to the color change observed in the irises of many infants, where initially lighter eyes gradually darken as melanin production increases. Through this method, the cycle of depigmentation and repigmentation provides a reversible, tailor-made solution for altering iris color, reflecting the complex balance of melanin presence within the eye.

The medication silences the OCA2 gene upon reaching the melanocytes in the iris stroma. Upon administration, the medication targets and inhibits the activity of the OCA2 gene specifically within the melanocytes located in the iris stroma. This inhibition leads to a reduction in OCA2 expression, which in turn negatively impacts the development and functionality of melanosomes. As the melanosomes mature less effectively and become less functional, the synthesis of pigment within the iris is diminished, resulting in a decrease in overall pigmentation. With a lightly pigmented stroma in the front chamber of the eye, light rays are scattered according to the laws of optics and reflect back to the observer as bright colors (for example blue and green). The produced medication consists of RNA therapeutic agent e.g. in the form of siRNAs or antisense oligonucleotides (ASOs), which have at least one complementary sequence to that of the human OCA2 mRNA. The sequence consists of 15 to 30 nucleotides selected out of the OCA2 mRNA molecule which in some humans comprises approximately 3,143 nucleotides e.g. as denoted by SEQ ID NO: 65. The RNA therapeutic agent does generally not activate the immune system, e.g., it does not increase cytokine levels, such as TNF-alpha or IFN-alpha levels.

### Example 5

Effects of siRNA Transfection Targeting OCA2 on Melanin Levels in B16-F10 Cells

### Melanin Absorbance Quantification

Across all spectra tested, linear regression was observed for melanin, with a slightly lower background at OD405 nm. It was decided to conduct melanin measurement in cells at absorbance wavelengths of 360 nm, 410 nm and 490 nm.

Cells were incubated with a suiting media according to ATCC recommendations at 37° C. with 5% CO2. Inspection and documentation (including pictures) were carried out at 24, 48, and 72 hours post-seeding. For the 96-well plate, viability was measured using the CellTiter-Glo assay. For the 6-well plate, cells were incubated up to 72 hours before collection for RNA extraction, RT-PCR, and qPCR setup.

Different methods were tested for absorbance either directly on cells, or after lysis, or in the media in different cells concentration, as further detailed below. The conditions for melanin measurement were calibrated using 3 different concentrations of B16-F10 (created by seeding 3,000, 7,500 and 15,000 cells in 24 well plates.) HEK-293 cells were seeded as well to serve as negative control cells (no-melanin).

Melanin absorbance measurements involved assessing melanin in various forms:
1. directly on cells;
2. in the medium, based on the method described in Chung, S., Lim, G. J. & Lee, J. Y. Quantitative analysis of melanin content in a three-dimensional melanoma cell culture. Sci Rep 9, 780 (2019);
3. intracellular after lysis in both 24-well and 12-well plates.

The correlation between the amount of cells seeded and the melanin levels was tested.

It was noted that B16 cells produce greater quantities of melanin upon achieving confluency (stressed).

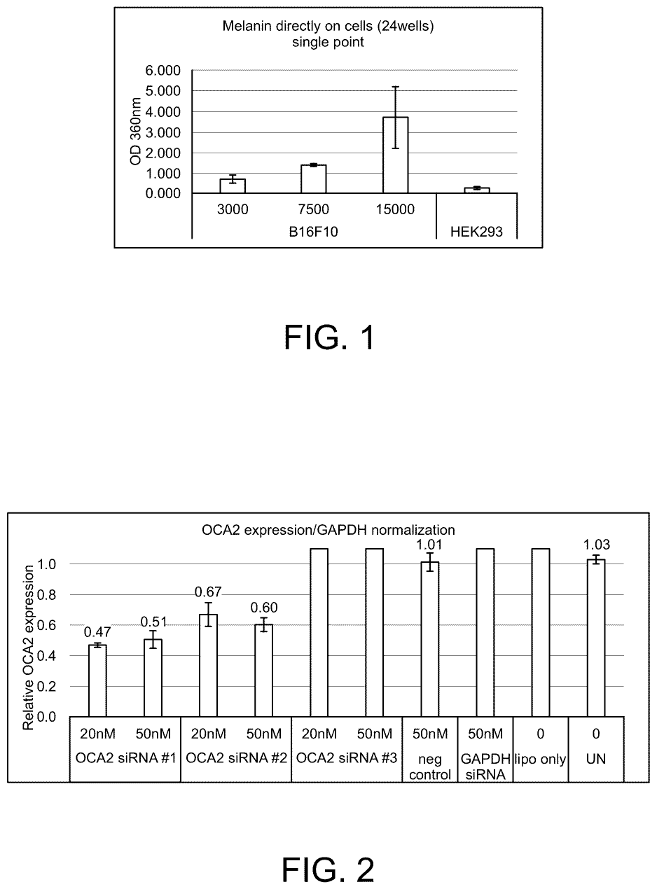

From all the three methods mentioned above, the only method that showed correlation between amount of cells and the absorbance on spectrophotometry was-directly on the cells as shown in FIG. 1. Since measurements in lysed cells and in the media didn't produce concentration dependent results, these methods were neglected. Further, measuring directly on cells is the most straightforward and less technically dependent, less “intervened” assay.

B16 cells were then transfected with siRNAs targeting the OCA2 gene. Three different sequences of siRNA targeting murine OCA2 in two concentrations (20 nM and 50 nM) were evaluated for their mRNA knockdown and melanin depigmentation effect on the B16 cells:

OCA2 siRNA #1-
Sense:
GAUCAUAUUUGAGAUUGUUTT
as denoted by SEQ ID NO: 70;
Antisense:
AACAAUCUCAAAUAUGAUCAG
as denoted by SEQ ID NO: 71.
OCA2 siRNA #2-
Sense:
CGAUGAUUCCUGUACUCCUTT
as denoted by SEQ ID NO: 72;
Antisense:
AGGAGUACAGGAAUCAUCGTA
as denoted by SEQ ID NO: 73.
OCA2 siRNA #3-
Sense:
GGUUGCUAAUUUUAGCUGATT
as denoted by SEQ ID NO: 74;
Antisense:
UCAGCUAAAAUUAGCAACCAG
as denoted by SEQ ID NO: 75.

Cells were transfected with the above mentioned siRNA molecules and the transfection efficiency was tested using FACS analysis.

48 hours post-transfection the cells were harvested, their RNA was extracted and Real-time PCR analysis was performed, including data export, melting curve analysis, and calculation of fold difference and normalized expression ratio using the 2-ΔΔCt method. RNA Extraction was satisfactory with: Absorbance ratios 260/280 between 1.8-2.0 and 260/230 between 2.0-2.2, and with non-template controls (NTCs) showing Ct values above 35. As shown in FIG. 2, Only transfection with OCA2 siRNA #1 resulted in adequate silencing of OCA2 expression in the cells.

Melanin Absorbance after OCA2 Knockdown

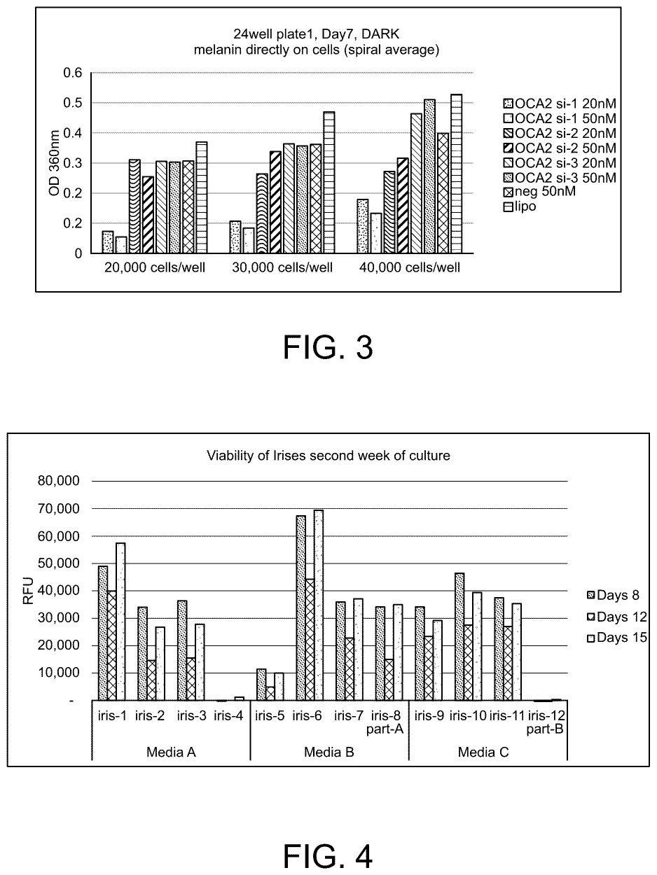

Melanin levels were assessed directly on cells. Correlated with OCA2 mRNA knockdown, OCA2 siRNA #1 showed melanin reduction using spectrophotometry in all 3 wavelengths: 360, 410 and 490. FIG. 3 shows melanin absorbance at OD 360 nm using the three OCA2 siRNA mentioned above. OCA2 siRNA #1, which significantly reduced gene expression, also led to a marked decrease in melanin levels, further substantiating the link between the silencing of OCA2 and the reduction of melanin levels.

### Example 6

Effects of siRNA Transfection Targeting OCA2 on Melanin Levels in Uveal Melanocytes

Primary melanocytes are isolated from a post-mortem human eye as follows: Tissue is obtained from eyes provided by an Eye Bank from donors who had consented that material could be used for research if not suitable for transplantation, in accordance with the tenants of the Helsinki Declaration.

Alternatively or additionally, uveal melanoma 92.1 cell line are employed. Cells are any uveal melanocytes: normal, malignant (such as uveal melanoma 92.1 cell line) or immortalized obtained by any method from any mammal species: primary cell line harvested or transformed iPSC or bought from a cell line supplier.

Transfection with the siRNA Drug

siRNA molecules are then added to the culture, along with a transfection agent. The siRNA molecules are purchased from Integrated DNA Technologies (IDT) and transfected in accordance with the manufacturer's instructions.

Isolated human primary uveal melanocytes are treated with siRNA-Lipofectamine RNAiMAX mixture in accordance with the manufacturer's standard protocol. Briefly, human primary uveal melanocytes are seeded in separate dishes. Upon incubation for 24 hours (cell monolayers at 60-80% confluence), cells are transfected with either OCA2-siRNA or siRNA-negative (scrambled siRNA). Initially, cells are washed with Opti-MEM GlutaMax medium (Invitrogen, Carlsbad, CA, USA) and then transfected with transfection mixture (diluted siRNA (separate):diluted Lipofectamine RNAiMAX::1:1) which is prepared in Opti-MEM GlutaMax medium with siRNA and Lipofectamine RNAiMAX, separately according to the manufacturer's instructions.

A concentration range of 1 to 100 nM is used for both negative control (as explained later under ‘control group’) and OCA-2-siRNAs. All wells/dishes have the same final volume. During transfection, the cells are cultured in the presence of the transfection mixture for 4-48 hours, and then the transfection mixture is replaced with fresh medium without antibiotics. Following 24 hours of incubation in fresh media, the cells are collected and examined or reseeded to continued culturing for examining the phenotypical change over longer period.

Preparation of Small Interfering RNA (siRNA)

Alternatively, for targeted knockdown of OCA2, OCA2 siRNA is synthesized by Invitrogen, Carlsbad, CA, USA, based on a transcript from the OCA2 gene (NCBI GenBank accession number, NM_000275.3). Exemplary nucleotide sequences are as follows: UAAAGAUUCCUGCUUUACAGA (sense strand, as denoted by SEQ ID NO: 67) and UGUAAAGCAGGAAUCUUUAGA (antisense strand, as denoted by SEQ ID NO: 2). Negative control RNA therapeutic agent is purchased from Thermo Fisher Scientific.

Analysis of mRNA Silencing

To quantify the silencing of OCA2 in iris melanocytes, qRT-PCR is performed on the treatment and control cultures. First, samples are prepared by lysing the cells and extracting the RNA. As a next step, RNA is reverse transcribed into cDNA using reverse transcriptase enzyme. Following the addition of specific primers for the OCA2 gene and a fluorescent probe, the qRT-PCR reaction is set up. A qRT-PCR machine is used to analyze the reaction, and data is collected. Alternatively or additionally, the RNA ligase-mediated rapid amplification of cDNA ends (RLM-RACE) method is used to detect the cleavage of specific genes as a result of OCA2-siRNA (kit purchased from Thermo-Fisher). In this procedure, the ends of the degraded mRNA are amplified and sequenced to detect the cleavage event of OCA2-mRNA.

As with qRT-PCR, total RNA is first isolated from cultured cells and then reverse-transcribed into cDNA using reverse transcriptase. RNA ligase is then used to ligate the cDNA with a specific adapter. The adapter is designed to bind only to the degraded mRNA produced by RNAi. A large number of copies of the degraded mRNA are generated by polymerase chain reaction (PCR) after ligating the cDNA. To identify the specific cleavage site of the targeted gene, the amplified cDNA is cloned and sequenced. Analyzing the sequencing data allows to identify the specific location and frequency of cleavage events.

### Analysis of Amount of Melanin:

A primary melanocyte is isolated and grown in culture and transfected in the same manner as described in the previous section. In order to harvest the cells after transfection, a trypsin/EDTA solution is added in order to detach them and harvest them. After harvesting, the cells are washed with PBS, suspended in extraction buffer, heated at a high temperature (70-100 C) for 0.5-2 h, and transferred to wells. A spectrophotometry device is used to measure the amount of melanin in a sample at 405 nm. A comparison is then made between the melanin content of the treated cells and that of the cells that were not transfected with siRNA as control cells.

Alternatively, in order to quantify melanin, the methods described above, i.e. in medium and directly on cells are employed.

Observation of melanin may be performed also in Light Microscopy.

### Transmission Electron Microscopy (TEM)

A next step involves examining the maturity stage of melanosomes in cells transfected with OCA2-siRNA using transmission electron microscopy, and comparing the average maturity stage of melanosomes in treated cells with the average maturity stage of melanosomes in control cells. This gives an indication of the effectiveness of the OCA2-siRNA molecule in reducing melanin production. This can be used to assess the potential of OCA2-siRNA as a depigmentation treatment for the iris.

### Pre-TEM Embedding:

In order to observe tissue through transmission electron microscopy (TEM), embedding is necessary for a number of reasons. Firstly, embedding protects the sample from damage during preparation and imaging, thus preserving the ultrastructure of the tissue. In addition, it enables the creation of ultra-thin sections which are necessary for TEM imaging. Finally, it provides structural support for the tissue sample, allowing it to be sectioned and mounted onto a TEM grid.

A description of a representative procedure is provided in the Experimental procedure section.

Once properly embedded, the samples are examined with a transmission electron microscope (TEM). Cells from each lattice grid are observed and recorded. Average melanosome maturation is compared between treatment and control groups.

### Viability Tests

A viability test is performed to determine whether OCA2-siRNA damages iridial melanocytes. Differentiation between living and dead cells is based on various mechanisms, such as the integrity of the membrane and normal metabolic activity.

The Trypan Blue staining method is employed: the dye penetrates the membrane of dead cells and stains them blue, while living cells are not stained. Using a microscope and hemocytometer, the percentage of viable cells is determined. By comparing the percentage of OCA2-siRNA treated living cells with the percentage of control viable cells, it is possible to determine the extent of the treatment toxicity.

In an alternative method, the MTT assay, the yellow compound MTT is added to the cells. It is metabolized by enzymes in living cells, changing its chemical composition and color to purple. By measuring the absorbance of certain light waves, spectrophotometry counts the number of live cells. After that, the absorbance of the OCA2-siRNA treated cells is compared with the control cells.

In a further alternative method, the viability is measured using the CellTiter-Glo assay.

### Example 7

### Ex Vivo Study—Viability of Murine Iris Explants

Murine iris explants were prepared as detailed above in the experimental procedure section. Ex Vivo Irises culturing and viability assessments were then performed along 3 weeks. It was previously shown that ex vivo harvested human irises could be cultured for prolonged durations (TENENBAUM E, KORNBLUETH W. Cultivation of Adult Human Iris in Vitro. AMA Arch Ophthalmol. 1958; 60 (2): 312-318; as well as Ringvold A, Nicolaissen B Jr. Culture of iris tissue from human eyes with and without pseudo exfoliation. Acta Ophthalmol (Copenh). 1990 June; 68 (3): 310-6). Their thin structured allows for sufficient nutrient diffusion to allow their survivability. In order to preform efficiency experiments on the irises, their survivability in culture, and best conditions were evaluated. Three different culturing rational medias were compared i.e. Media A, B and C (as detailed in the experimental procedures) for their ability to maintain iris viability over time.

Initially dozen irises were harvested and were grouped into 3 groups of 4 irises and cultured in one of the medias (A, B or C) for the entirety of the experiment. To replenish nutrients and remove waste, media was replaced every 2-3 days. For handling comfort, irises were taken out from their wells for media replacement using inserts (Millicell Hanging Cell Culture Insert, PET 0.4 μm, 24-well (Mercury); Catalogue: PTHT24H48).

After iris harvesting, the irises were placed on the inserts in 24-well plates. The wells contained media and stored cold during delivery.

Brightfield microscopy was employed to capture iris features. Irises were inspected for their morphology and cellular detachment daily under the microscope and pictures were taken.

Viability assay was performed using Presto Blue reagent.

Fluorescence intensity was measured at gain 1100. No significant difference in viability was seen between the different media (A, B and C-described above) along 3 weeks, as shown in FIG. 4. Fluorescence measurement of this assay was more sensitive than absorbance. Irises maintain most of their viability throughout 3 weeks of culturing.

### Transfection and Fluorescent Microscopy

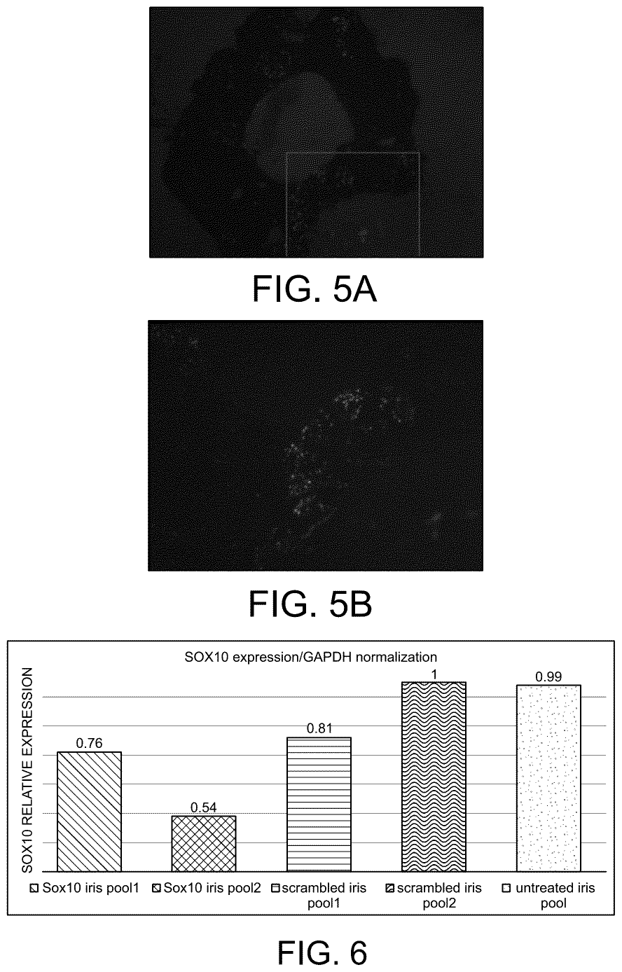

All irises were cultured in accordance with the established protocol detailed earlier, utilizing Media A for cultivation. The study incorporated eight iris samples sourced from black mice for the transfection process. The irises were transfected with 50 nM fluorophore-conjugated control siRNA, BLOCK-iT™ Fluorescent Oligo as RNAi Transfection Control by Thermo-Fisher & a costumized scrambled siRNA conjugated to fluorophore by IDT. To establish a baseline for comparison, two additional samples were designated as negative controls, undergoing transfection with Lipofectamine alone, devoid of siRNA. Fluorescence microscopy was employed to assess the samples at 20 hours post-transfection. The transfected irises exhibited successful uptake of fluorophores, which demonstrated colocalization with Hoechst, a nuclear stain, as illustrated in FIG. 5. This colocalization facilitated the identification of cellular positions and substantiated the feasibility of the transfection process. In contrast, the negative controls failed to exhibit any fluorescence, confirming the specificity of the fluorescence signal to the transfection procedure.

### Example 8

### Ex Vivo Study—Targeting the SOX10 Gene as Surrogate

OCA2 is expressed in both the stromal melanocytes and the Iris Pigmented Epithelium (IPE) of the iris. Consequently, treating the iris with OCA2 siRNA and observing a reduction in OCA2 expression does not necessarily indicate that the reduction is occurring in the target cells (stromal melanocytes). Thus, a decrease in OCA2 expression does not automatically signify successful delivery to the intended target cell population. Furthermore, the IPE constitutes a predominant cell type in the murine iris and accounts for a significant portion of the iris. As a result, a reduction in OCA2 expression in the relevant stromal melanocytes may not be detectable, even if it is achieved exclusively in the target cell population, due to the overshadowing effect of the high OCA2 expression from IPE cells. This is why a delivery-gene-surrogate was employed.

Sox10 is a gene expressed in neural crest-derived cells, including melanocytes.

It appears that in the iris tissue, the only cell type that express Sox10 are the target cells (stromal melanocytes) (Mori T, et al. SOX10 Expression as Well as BRAF and GNAQ/11 Mutations Distinguish Pigmented Ciliary Epithelium Neoplasms From Uveal Melanomas. Invest Ophthalmol Vis Sci. 2017 Oct. 1; 58 (12): 5445-5451).

Silencing SOX10 with siRNA and then observing a reduction in its expression in the iris is a method, originally employed here to see a successful siRNA transfection and function in the stromal melanocytes. Assuming that if SOX10 was able to be silenced, a OCA2-siRNA molecule proven to exert an effect in vitro on melanocytes of the same species would silence OCA2 in stromal melanocytes.

Mouse iris tissues were cultured and used in transfection experiments. SOX10 and scrambled siRNAs, each at a concentration of 50 nM, were transfected into the iris tissues using Lipofectamine RNAiMAX transfection reagent, following the manufacturer's guidelines. The irises were then incubated at 37° C. Media exchange was carried out 24 hours post-transfection. Finally, the irises were collected for RNA extraction 48 hours post-transfection. Additionally, a pool of irises that did not undergo any transfection was also harvested for their RNA to serve as an additional control.

RNA was extracted from the irises, as described above in the experimental procedures. The RNA samples were then reverse-transcribed to cDNA using a standard protocol.

Afterwards, quantitative PCR was carried out to assess the expression levels of GAPDH, and SOX10 genes.

No-template controls (NTC) as well as negative controls for the reverse transcription (Neg ctrl RT) were included to ensure the specificity and reliability of the qPCR results.

Expression Quantification: The fold difference in gene expression was calculated using the 2-ΔΔCt method, with normalization to a reference gene, GAPDH, and comparison to control samples to determine relative expression levels of SOX10. FIG. 6 illustrates SOX10 expression of iris samples following SOX10 siRNA transfection.

### Viability Under Light Exposure

Ex vivo iris tissues with comparable viability metrics were cultured overnight (O.N.), in a standard incubator. The irises were cultured in two separate culture plates: one exposed to a 9W LED light panel placed 8 cm away, simulating environmental light exposure, and the other located at a distance from any direct light source to serve as a control. Temperature measurements were taken throughout the experiment to ensure safety for the irises.

The viability of exposed tissues did not show any significant decrease after overnight exposure to light. For tissues exposed to light, fluorescence viability readings, after 2.5 hours in presto blue, ranged from 15,708 to 36,304 Relative fluorescence units (RFU), with an average viability of 25,490±8,007 RFU. In comparison, tissues not exposed to light showed viability readings ranging from 12,990 to 31,942 RFU, with an average viability of 24,876±6,724 RFU. Overall, the average viability for all samples was 25,207±7,141 RFU. The experiment concluded that there was no significant loss in viability of ex vivo iris tissues cultured overnight with exposure to 9W LED light panel placed 8 cm away, as compared to those not exposed.

### Example 9

### OCA2 Depigmentation Study Utilizing Murine Iris Samples

Nine murine iris samples, harvested as detailed earlier in this document, were employed for the study. These irises were sustained in Media A and incubated according to the protocols described herein, with media replacements conducted every 2-3 days, as in the studies described earlier, to preserve their viability. Viability assessments conducted thrice weekly revealed that the irises sustained their viability throughout the study. There was no significant difference in viability between the treatment groups, indicating that the treatments were well-tolerated. The same OCA2 siRNA sequence proven to reduce OCA2-mRNA levels by over 50% in vitro within the B16 melanoma cell cultures from Example 5 above, was used [OCA2 siRNA #1—Sense: GAUCAUAUUUGAGAUUGUUTT as denoted by SEQ ID NO: 70; Antisense: AACAAUCUCAAAUAUGAUCAG as denoted by SEQ ID NO: 71.]

Transfection of the irises commenced on day 1, with subsequent transfections on days 4, 8, and 11 (total of 4 transfections, every 3-4 days). Each iris received the treatment it was allocated for, consistently, for the entirety of the experiment (all 4 transfections). 6 irises received OCA2 siRNAs either 50 nM or 100 nM (‘Treatment Group’), and 3 other irises received scrambled siRNAs at 100 nM (‘Control Group’). To mitigate positional bias within the experimental setup, the groups were distributed across the plate in a manner that interspersed the different groups. Lipofectamine RNAiMAX was utilized for each transfection, according to manufacturer recommendations; Irises were incubated for 24 hours before media replacement to remove any remaining transfection reagents or siRNA.

Illumination conditions for the iris samples were meticulously controlled as detailed in the Experimental procedure section.

Imaging was performed at baseline and at day 14 of the experiment (3 days after the 4^{th} transfection and immediately before fixating and processing for histological evaluation) using a Panasonic Lumix G9 camera equipped with a Laowa 25 mm f/2.8 Ultra Macro 2.5-5× lens. as detailed in the Experimental procedure section.

Following comprehensive efforts, the observable outcome in the images showed a dramatic change in the coloration of the OCA2-siRNA treated irises, while no such change was seen in the control group (FIG. 8).

The visual analysis revealed a pronounced and significant alteration in the iris coloration of the treated subjects (see FIG. 8A-8B, clear differences were observed especially in the periphery of the iris), in stark contrast to the unchanged iris coloration in the negative control group (see FIG. 8C-8D, no dramatic change if any was observed, including the periphery).

### Imaging Analysis

The pictures were further analyzed using the ImageJ software. The iris area within the frame was carefully masked, allowing for the specific analysis of pixels contained within its boundaries, from which the average color saturation was determined. The background of the iris, which was the same white diffuser (cyngustech) was consistent across all images, and was measured for its color-saturation in each image, which could vary between 0 to 255 for each pixel, O having no color-saturation and 255 having the highest color-saturation. Then for each iris in each image the color-saturation was normalized by subtracting the color-saturation of the background. Thus, maintaining standardization between all the pictures. Color-saturation value from all the images for a specific iris on both, baseline photo shooting and day 14 photo shooting were averaged to enhance accuracy even further. A pre-treatment and a post-treatment color-saturation value for each iris was calculated as such.

At the work published by Andersen et al, 2013 (Andersen J D, et al. Genetic analyses of the human eye colors using a novel objective method for eye color classification. Forensic Sci Int Genet. 2013 September; 7 (5): 508-15), the investigators analyzed images of different iris colors and used the HSV color space to measure the eye color. The S component of the HSV color space separated the blue and brown areas of the iris efficiently, and showed the largest difference in pixel value between blue and brown region. They concluded that color-saturation may be preferred in quantitative eye color investigations.

The blue color in blue eyes comes mainly from the Rayleigh scattering of light. This scattering effect dilutes the color intensity, as more wavelengths are mixed in, resulting in lower perceived color-saturation. There is less melanin present to absorb light, so the color is less vivid (less color-saturation).

Brown irises have more melanin than blue ones, but not such that all light is absorbed. This optimal level of melanin allows brown irises to absorb enough light to reduce scattering, while still reflecting enough light to exhibit the natural color of the melanin. The result is a more intense, vivid color, and thus a higher perceived color-saturation.

In the article of Liu et al., 2010 (Liu F, et al. Digital quantification of human eye color highlights genetic association of three new loci. PLOS Genet. 2010 May 6; 6 (5): e1000934), it was mentioned that digitally extracted iris (eye) color was quantified into two interval dimensions hue (H) and color-saturation(S) while on the figure in the article clearly presenting brown irises has more saturated compared to blue ones.

With an abundance of melanin, black irises absorb the vast majority of light, leaving very little to be reflected back to the observer. This absorption results in a lack of perceived color, thus linking black with the notion of low or no color-saturation in terms of color properties.

In an analysis performed for pictures of different eye colors, irises of both colors, black (very dark brown) or blue, were significantly less saturated compared to brown irises which were the most saturated of all the images analyzed. In conclusion brown irises are expected to have higher color-saturation compared to black (or very dark brown) irises. On the spectrum between 100% black (100% absorption) to a brown object, the more an object is nearer to complete black, the less saturated it is, and vice versa.

The results of the image analysis are presented in detail in Table 1. Irises A-F were allocated to OCA2-siRNA treatment while X, Y and Z received shambled siRNA. The average color-saturation (normalized by the background) for groups was almost identical at baseline (48 vs 49 out of 255). At the post images for the irises there was a clear distinction between the 2 groups (67 vs 53).

TABLE 1
Saturation of Iris Normalized by Background Saturation.
Saturation of Iris Normalized by Background Saturation

| Irisx | Pre- | | Post- | | | |
|---|---|---|---|---|---|---|
| # | Treatment | Average | Treatment | Average | % change | |
| A | 56.76 | 48.30 | 70.68 | 67.19 | 24.52% | 41.31% |
| B | 39.61 | | 60.68 | | 53.18% | |
| C | 48.21 | | 69.08 | | 43.29% | |
| D | 40.13 | | 69.93 | | 74.26% | |
| E | 52.38 | | 62.60 | | 19.51% | |
| F | 52.71 | | 70.18 | | 33.13% | |
| X | 39.675 | 49.42 | 56.90 | 52.90 | 43.42% | 10.78% |
| Y | 57.71 | | 48.08 | | −16.69% | |
| Z | 50.87 | | 53.73 | | 5.61% | |

To calculate the percentage change in iris color saturation for each iris, the following formula was applied:

$$\% change = \frac{post treatment - pre treatment}{pre treatment} \times 100$$

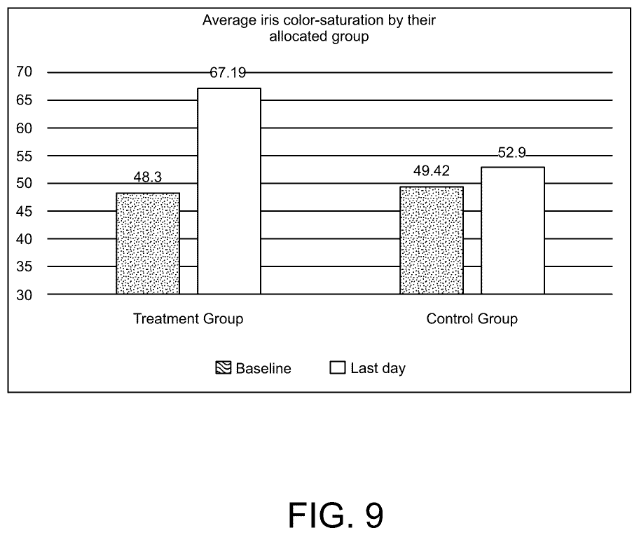

The average color-saturation change following the experiment showed a clear tendency for increased color-saturation in the treatment group over the control group, as shown in FIG. 9.

### Histological Evaluation:

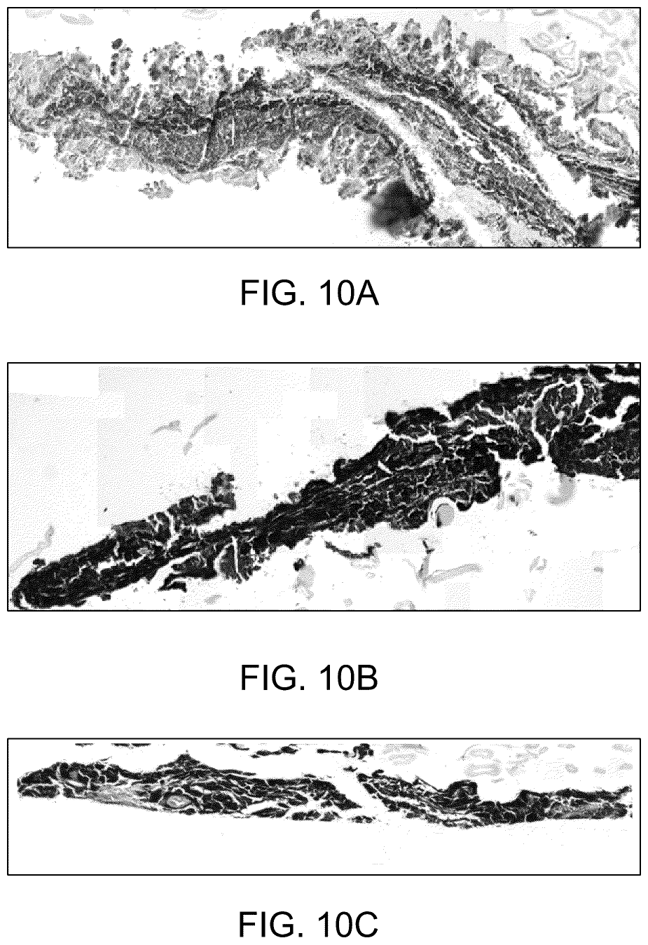

As the color saturation of irises treated with OCA2-siRNA increases—a phenomenon associated with brighter brown irises—histological analysis reveals that these irises exhibit less pigmentation compared to those treated with scrambled siRNA and subjected to identical photobleaching conditions. A representative microscopic image is provided in FIG. 10.

Control-treated irises displayed no evidence of depigmentation (FIGS. 10B, 10C). The histological condition of these tissues is analogous to that of an untreated (nor photobleached, FIG. 10C) DBA/2J mouse iris, which was fixated and observed microscopically hours post-dissection without any intervening treatment.

### Example 10

### Ex Vivo Study—Depigmentation of Human Iris Explants

Human irises are isolated from a post-mortem human eye as follows: Tissue is obtained from eyes provided by an Eye Bank from donors who had consented that material could be used for research if not suitable for transplantation, in accordance with the tenants of the Helsinki Declaration.

Eyes are enucleated and the uveal tract dissected out (a circumferential incision is made in the sclera 8 mm behind the limbus). The anterior portion of the globe, including the anterior sclera, lens, iris, and ciliary body are excised and placed in a culture dish). Extraction of the iris at the root is followed by placing the iris in a culture dish with its posterior surface facing downward. The cells are cultured in Media A in 24, 12 well plates or bigger. Media is replaced every few days.

Viability is evaluated every few days in Presto Blue and under a microscope.

Transfection efficiency is evaluated as described in the murine ex vivo with fluorescently target siRNA such as Cy5 and then viewed by Fluorescence and Confocal microscope.

SOX10 knockdown qPCR is performed as described in murine ex-vivo.

Depigmentation assay is performed as described in murine ex-vivo.

Melanin reduction is measured through image analysis (as described herein), histological examination, or any other available methods designed to quantify melanin levels. Following the initial depigmentation phase, the experiment extends by ceasing the OCA2 silencing treatment while continuing to maintain the iris explants alive in Media A. This cessation allows for the observation of the irises as they potentially regain their pigment, aiming to document the process and rate of repigmentation back to their original pigmentation levels. The assessment of repigmentation includes monitoring melanin synthesis over time through image analysis, histological examination, and other melanin quantification methods, similar to the initial depigmentation assay.

### Example 11

### In Vivo Study—Targeted Depigmentation of Iris Stromal Melanocytes in Mammals

A rodent (mouse or rat), or a leporid (e.g., New Zealand rabbit, Dutch Belted rabbit, or European rabbit), a primate (e.g., cynomolgus monkey), or a suid (e.g., domestic pig), or any other mammal is treated with either intraocular injections or application of eye drops of OCA2-siRNA. As described herein, the molecule is designed according to the specifications (see further details below concerning siRNA sequences). In this procedure, a mammal is anesthetized with anesthetics, such as ketamine (5 mg/kg) and xylazine (5 mg/kg), and then treated with active ingredients at concentrations of 0.1% up to 6% concentration and at a volume proportional to the mammal eye size.

OCA2-siRNA specific for mammals is designed to match their OCA2 mRNA. For example, the mRNA sequence of OCA2 in rabbits was retrieved from the GenBank database of the National Institutes of Health (>XM_008269748.3 PREDICTED: Oryctolagus cuniculus OCA2 melanosomal transmembrane protein (LOC100340522), transcript variant X1). Exemplary nucleotide sequences are as follows: GCAUCUAGAGAACAAAGAUGG (sense strand, as denoted by SEQ ID NO: 68) and AUCUUUGUUCUCUAGAUGCAU (antisense strand, as denoted by SEQ ID NO: 69). Further optimization are made to design the most efficient sequence for silencing as further detailed below.

Animal subjects are injected intravitreally or intracamerally as described previously (Kodjikian L, et al. (2010) Invest Ophthalmol Vis Sci. 51 (8): 4125-32) with a mixture containing OCA2-siRNA naked/conjugated/mixed with transfection reagent and/or SOX10-siRNA (for surrogate experiment, fluorophore conjugated RNA, such as BLOCK-iT for fluorescent biodistribution experiment). An intraocular injection of a negative control (scrambled siRNA that was checked to not target any mRNA of that species) is given to the contralateral eye or to the control group, negative controls could also be untreated or treated with PBS or with a carrier alone.

### OCA2-siRNA is Efficiently Taken Up by Iridial Melanocytes In Vivo

Following intracameral administration of OCA2-siRNA, fluorescent Cy5-labelled siRNA is injected into the iris. The iris is harvested hours to days after and the fluorescence distribution is evaluated either by fluorescence/confocal microscope. Alternatively, the ocular tissue is broken up to single cells and then sorted for their fluorescence by fluorescence-activated cell sorting (FACS) analysis hours to days later. The presence of Cy5 staining is consistent with siRNA being taken up by the cells. Cy5-labelled siRNA is injected intracamerally and then spreads into iridial cells/melanocytes and the fluorescent signal within these cells is measured hours after injection and the duration of the fluorescent signal in these cells is assessed. Cy5 fluorescent dye, when being taken up by the cells, produces a bright fluorescent signal. This signal can then be measured and used to determine if siRNA has been picked up by the cells.

To strengthen the findings, in situ hybridization is also used to detect OCA2-siRNA in the iris with a radiolabelled oligonucleotide probe complementary to the guide strand of OCA2-siRNA. By using the ‘Stem and Loop’ qPCR method after a single intracameral injection, the inventors measure how long after injection OCA2-siRNA is still detected in the iris and in which cells in the tissue.

Additionally, to assess the biodistribution of fluorophore-labeled siRNA in the iris, in living subjects, mammals receive the siRNA via eye drops or intraocular injection. After administration, subjects are anesthetized with isoflurane (1.5-2%) in oxygen at a flow rate of 1.5 L/min. The IVIS Spectrum imaging system (Perkin Elmer, UK), set to the specific filter settings for the fluorophore, is used for imaging the iris at predetermined intervals. Analysis of fluorescence intensity and distribution is conducted using LivingImage software, with regions of interest (ROIs) consistently defined within the iris across all subjects. This approach allows for quantitative evaluation of siRNA distribution, critical for understanding its delivery and localization within the ocular tissue.

OCA2-siRNA Reduces OCA2-mRNA in Treated Eyes and/or SOX10 as a Surrogate

Irises are dissected (see Ex-vivo dissection) from OCA2-siRNA- and control-injected eyes a few hours to days after injection, are broken down (homogenized) via beads. RNA is extracted and eventually (see Ex-Vivo RT-PCR) qPCR analysis is performed to demonstrate that OCA2-siRNA reduces target mRNA expression in treated eyes. A comparison is made between the expression of OCA2 mRNA levels in OCA2-siRNA treated animals and control animals. SOX10, a surrogate gene, is also silenced and measured to demonstrate that silencing precisely occurs in the melanocytes. The iris is made up by different kinds of cells, of them, both the stromal melanocytes and the iris pigmented epithelium express OCA2 (both synthesize melanin in melanosomes). But only the stromal melanocytes express SOX10. Knockdown of SOX10, a gene only expressed in melanocytes within the iris proves not only the transfectability of the target cells but that siRNAs are efficient within these cells and able to utilize the cell machinery (i.e. RISC) to affect gene expression.

### OCA2-siRNA Induces Specific RNAi-Mediated OCA2-mRNA Cleavage In Vivo

To verify that siRNA activity was specifically induced by OCA2-siRNA in animal irises after intravitreal administration, the RNA ligase-mediated rapid amplification of cDNA ends (RLM-RACE) method is used to detect the presence of OCA2-specific cleavage products in animal irises.

The RACE products generated from OCA2-siRNA-injected eyes and eluted from the gel are then cloned and sequenced to determine if cleavage occurred at the predicted site in the OCA2 mRNA. To demonstrate that only OCA2-siRNA can cleave OCA2-mRNA, the same analysis is conducted on sequenced clones obtained from vehicle-treated eyes (the negative control) and OCA2-siRNA-treated eyes using the same procedure, in order to verify that no OCA2-siRNA-mediated cleavage was detected.

### Light Source & Depigmentation:

It is submitted that the melanin in stromal melanocytes is constantly exposed to light, and goes through photobleaching (always, but faster with light exposure) and synthesized to maintain pigment equilibrium. Eye brightening throughout life is rare and when it occurs, it is usually associated to some pathology, such as Fuchs heterochromic iridocyclitis and even some cases of Horner's syndrome (Beynat J, Soichot P, Bidot S, Dugas B, Creuzot-Garcher C, Bron A. J Fr Ophtalmol. 2007 September; 30 (7): e19) since the melanocytes are actively synthesizing melanin. It was shown that even in stromal melanocytes harvested from adult human irises new melanosomes are created (Hu D N, McCormick S A, Orlow S J, Rosemblat S, Lin A Y, Wo K. Melanogenesis by human uveal melanocytes in vitro. Invest Ophthalmol Vis Sci. 1995 April; 36 (5): 931-8.). Photobleaching is a physical phenomenon that cannot be prevented from the first line of pigment cells in a tissue directly exposed to photons.

Therefore, determining the light exposure of subjects during depigmentation with OCA2-siRNA affects the depigmentation duration. It is known that photobleaching occurs at high rate with light exposure compared to dark conditions (Mokrzyński K, Sarna M, Sarna T. Photoreactivity and phototoxicity of experimentally photodegraded hair melanosomes from individuals of different skin phototypes. J Photochem Photobiol B. 2023 June; 243:112704). Humans differ significantly in the amount of exposure to daylight-depending on geographic location, lifestyle, occupation, season, cultural practices and other parameters. Beside daylight, humans are also exposed to artificial light or indoor lighting during their active time, and the joules of this light differs a lot. Therefore several lighting conditions are tested during the experiment, from which, recommendations for light exposure for users who wish to safely expedite their depigmentation without causing harm, could be determined.

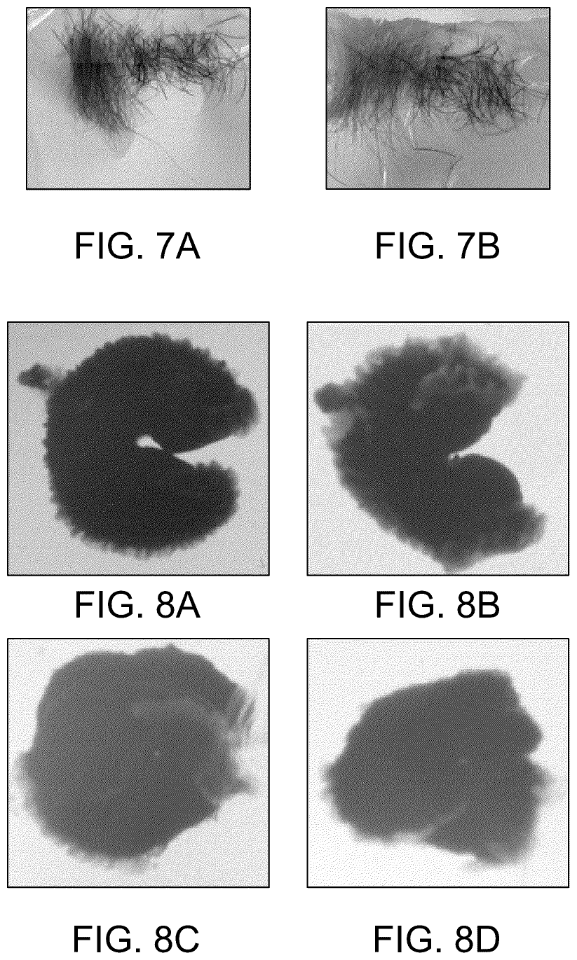

On the initial day of the investigation, inventors started with the placement of human scalp hair, uniformly cut from donations by a young female and a young male. The male's hair exhibited a darker pigment, characterized as dark brown to black, whereas the female's hair was of a lighter brown shade. Prior to the collection, the hair was only exposed to standard cleansing procedures with shampoo and conditioner, without exposure to any chemical treatments. The samples were divided into two groups, each containing strands from both individuals, and simultaneously introduced to distinct environmental conditions. The control group was stored in an environment shielded from light, whereas the experimental group was subjected to photobleaching, positioned less than 10 centimeters from a 9W 6500K LED light source. This same lighting setup was later employed in an ex-vivo depigmentation study. Temperature measurements were meticulously recorded throughout the duration of the experiment to ensure that the thermal output of the lamp did not influence the surrounding environment, confirming the efficiency of the LED light in minimizing heat production. Observations on the tenth day revealed minimal alterations, yet by the twenty-second day, significant differences in pigmentation were notably evident in the female's hair specimen (see FIG. 7A, 7B). This experiment was pivotal in calibrating the optimal light intensity required to induce melanin degradation.

The photobleaching phenomenon aligns with observations in individuals possessing lighter hair shades, which tend to lighten during summer months and darken during winter, a process previously documented in various studies suggesting that LED light can precipitate depigmentation. It is important to note that hair strands lack living cells and are incapable of melanin synthesis over time. However, living pigmented tissues, such as the iris, maintain melanin homeostasis. In individuals with darker irises, exposure to light triggers melanin degradation within the stromal melanocytes—a process absent or significantly reduced in individuals with light-colored eyes. Despite this, melanocytes continue to synthesize new melanin, ensuring that, for instance, the brown-eyed individuals do not experience a lightening of eye color upon sun exposure. A change in eye color is typically indicative of pathology, according to the American Academy of Ophthalmology (AAO). Yet, light exposure can serve as a catalyst for pigment reduction in cases where melanin synthesis is intentionally inhibited.

Furthermore, it has been demonstrated that melanin degradation is expedited under light exposure compared to darkness. Any form of light exposure, whether from the sun, ambient sources, or therapeutic lights, can accelerate pigment reduction, potentially facilitating the lightening of tissue color, including the transition from brown to blue eyes. Additional methods that may accelerate melanin breakdown-coupled with the inhibition of new melanin production such as pharmacological treatments or behavioral interventions, e.g. prolonged water fasting, or the utilization of animal models genetically predisposed to higher rates of autophagy or expedited melanin degradation capabilities.

### Phenotypic Analysis

Phenotypical change in iris color is evaluated by the following methods:

Following the injection of OCA2-siRNA, the depigmentation effect of the siRNA is further evaluated. Examination of the animal eyes is conducted to determine whether anterior iris pigmentation has been lost throughout the entire surface of the iris, both overall and punctate. The purpose of this is to determine the pattern and distribution of the downstream effects of silencing. Each day, pictures are taken of the eyes treated with OCA2-siRNA and untreated eye or eyes treated with negative controls (PBS/or scrambled siRNAs). The color change of the eye is observed visually (or with computerized software analysis such as described previously (Andersen J D, et al. (2013) Forensic Sci Int Genet. 7 (5): 508-15) in the injected eye (with siRNA) and compared to the control eye (with control scrambled siRNA). This method allows to empirically quantitate the eye color and its change thereof. The amount of pigmentation is evaluated periodically (every few hours or every few days) throughout the study. Furthermore, the length of time it takes for the iris to become pale with virtually no pigment is evaluated. Afterwards, measurements are continuously taken to determine whether the effect persists, reverses, or if any other changes have occurred. Repigmentation duration is also evaluated. Together with time to pale—these durations helps determine future therapy treatment regimen: Treatment interval for depigmentation (light eye achievement), maintenance interval (maintain the achieved effect—could be anywhere on the human eye color spectrum e.g., brown-greenish) and the time it takes a user to revert back to his/its original eye color after treatment cessation (natural melanin aggregation in the irises—similar process happening on babies born with blue eyes that turn brown in the course of several weeks to months).

### Analysis of Iris Pigmentation Under Microscopy

In order to further evaluate the reduction in iris pigmentation, the animals are euthanized and their eyes are enucleated. Then, a thorough histological examination of the tissues of the animals via light microscopy and/or transmission electron microscopy is conducted.

Transmission electron microscopy (TEM) is used to evaluate the maturation stage of melanosomes in the irises of the animals. The percentage of melanosomes located at stages I-II in the OCA2-siRNA treated animal iris stroma is compared to the control animal iris stroma. A comparison is also made between the percentage of melanosomes at stage IV in the treated animal iris and the percentage in the control animal iris. The IPE and RPE are also evaluated to determine whether loss of pigmentation has occurred there as well. In this manner, it is possible to determine if the treatment has affected the melanosomes in the iris stroma and whether it has altered the amount of pigmentation in the other vital ocular tissues.

### Quantification of the Amount of OCA2 Protein:

The iris is homogenized in lysis buffer and the total protein is measured using a Bradford protein assay (Roche, Germany). A normalization of the samples for total protein is performed prior to the ELISA assay for the animal OCA2. This is done in order to ensure that the amount of OCA2 in each sample is accurately measured. An ELISA assay is then used to compare protein levels of OCA2 in animals injected with OCA2-siRNA and control animals.

### Adverse Effects

A slit lamp examination is conducted at baseline, followed by electroretinography. The Slit lamp examination is used to examine the structures of the eye, such as the cornea, iris, and lens. It can detect signs of disease or damage. Electroretinography is then used to measure the electrical impulses generated by the cells at the back of the eye, which helps to diagnose certain eye conditions. A series of ophthalmic examinations are conducted throughout the study in order to assess adverse effects as well as capture any changes in the eye.

During the study, all other parameters (including body weight) of the animals are measured. The cornea and iris are inspected for the presence of pathological changes. The anterior chamber is examined for inflammation and flare. An examination of the lens is conducted to determine its transparency. The conjunctiva, sclera, and posterior segments of the eye are thoroughly examined. Additionally, OCT is used to detect structural changes in the eye The purpose of this examination is to determine whether any changes have occurred in the eye due to the drug, such as triggering inflammation, inducing infection, causing cataracts, glaucoma, or any other ocular abnormality.

During the study, the fur of the entire animal is examined for hypo- or depigmentation. Additionally, skin samples are collected and examined under a microscope in order to assess the maturation of melanosomes in epidermal melanocytes. Depigmentation and retrogression of the overall melanosome stages are intended to be determined. If they have occurred, this may indicate that OCA2-siRNA inhibits expression of OCA2 in hair follicles, which implies systemic effects or spillage of topical treatment near the eye and eyelids.

### Example 12

### Eye Drops Formulation Screening for Optimized Stromal Melanocytes Biodistribution

It is imperative that the formulations being tested not only match the physiological pH for compatibility but also demonstrate stability over time to ensure the active ingredient effectiveness is maintained.

Formulations that provoke irritation can lead to increased tear production and potential tissue damage as the eye attempts to counteract such irritation. This reaction can significantly impact the biodistribution of ocular drugs, underlining the importance of developing non-irritative formulations from the early stages of in vivo experimentation.

Viscous compounds, including gels, are notable for their prolonged retention within ocular tissues such as the conjunctivas, offering a promising avenue for extended drug delivery. The investigation encompasses both viscous formulations, with consistencies up to that of Vaseline, and aqueous solutions, in conjunction with an analysis of commercially available eye drops. This multifaceted approach also involves the reverse engineering of market products and a thorough review of literature for both deliberate and incidental reports of drug distribution to the iris. Excipients sanctioned by regulatory bodies like the FDA (Food and Drug Administration) and EMA (European Medicines Agency) for ocular applications are preferred due to their established safety profiles and regulatory familiarity.

To enhance the duration of the drug presence on the ocular surface, viscous materials are considered for their retention capabilities. However, these substances may restrict the release of siRNA molecules. Given that siRNA is water-soluble yet requires penetration through lipid barriers to effectively reach intraocular targets, the exploration of various lipid-based liquids or bi-phasic formulations might provide a viable pathway. The application of tight junction penetrators, such as Benzalkonium chloride (BAK), within the confines of approved concentrations, could further enhance the permeability of siRNA through critical ocular barriers, including the cornea, conjunctiva, sclera, ciliary body, and iris. This strategic approach underscores the necessity of a delicate balance between formulation stability, compatibility, and permeability to optimize ocular drug delivery.

A rodent (mouse or rat), or a leporid (e.g., New Zealand rabbit, Dutch Belted rabbit, or European rabbit), a primate (e.g., cynomolgus monkey), or a suid (e.g., domestic pig), or a canine (e.g., domestic dog) or any other mammal is treated topically with eye drops containing OCA2-siRNA complementary (partially or completely) to subject specie mRNAs, capable of knocking down its OCA2 expression.

The formulation containing siRNA (hereafter referred to as “the tested drug”) is administered in this in vivo experiment through topical ocular delivery strategies to enhance its anterior chamber distribution and bypassing the ocular barriers. One approach involves immersing an optical contact lens in the tested drug and then applying the lens onto the eye. In a different approach, the tested drug in directly placed on the ocular surface, followed by the application of a contact lens to maintain the formulation position, and prevent dispersion due to tear fluid dynamics. Yet, in another method the animal subject is sedated to ensure the eye remains open, allowing for the precise placement of the drug drop and preventing involuntary blinking or movement from dispersing the medication. The tested drug is also applied directly to the eye, with subsequent contact with a glass surface to leverage surface tension in keeping the liquid formulation from spilling away.

Repeated administrations of the eye drop may be considered several times a day, though it is limited to no more than twice daily when involving sedation, to reduce stress and potential harm to the animal. Prolonged contact of the drop with the external part of the eye is attempted, ranging between 5 to 20 minutes, to facilitate optimal drug absorption and bioavailability. The treatment is either unilateral or bilateral.

Numerous formulations containing active ingredients are applied topically as eye drops, as detailed above. Following application, a bioanalytical assessment is conducted for each formulation to evaluate their delivery effectiveness, employing methodologies similar to those described in previous examples. The analysis includes:

The use of conjugated markers, such as fluorophore-conjugated siRNA, beta-galactosidase, or other agents capable of illustrating tissue and cellular distribution under microscopic examination 24 hours post treatment. A consideration is taken for the potential impact of these conjugates on the pharmacokinetics of the entire molecule.

Formulations containing either OCA2-siRNA or SOX10-siRNA as the active ingredients are administered. 48 hours after treatment, the iris is harvested, and its RNA is extracted and analyzed using qPCR (as previously described) to evaluate any knockdown effects. This analysis helps to determine the biodistribution conferred by the formulation.

Application of in-situ hybridization techniques to further investigate the tested-drug delivery to the tissue as well as utilization of IVIS imaging, despite its lower resolution, to garner overarching insights into the general distribution patterns of the formulations.

These methodologies collectively establish a comprehensive framework for evaluating the biodistribution of each eye drop formulation and identifying the optimum.

Through this process, the optimal eye drop formulation is subject to rigorous screening to assess its compatibility with the active ingredient, as well as a thorough evaluation of biodistribution and pharmacokinetic properties. This assessment includes a detailed examination of chemical attributes, such as pH levels, to ensure the formulation safety and stability. This, in turn, guarantees its efficacy and appropriateness for use in the eye. Additionally, the potential for scaling up production efficiently is carefully considered.

In the present example, a basic eye drop formulation comprising purified water, benzalkonium chloride, sodium chloride, hydroxypropyl methylcellulose, and sodium phosphate buffer is being tested for its ability to deliver OCA2-siRNA to the eye. The selection of these components is based on their established use in ocular applications, focusing on ensuring the formulation compatibility with ocular tissues and the effective distribution of siRNA within the eye.

### Example 13

### Phase 0 Clinical Trial

A Phase 0 clinical trial is conducted to evaluate the safety, tolerability, pharmacokinetics and pharmacodynamics of micro-doses of topically applied eye drops containing OCA2-siRNA. Ten to fifteen healthy human volunteers are recruited, preferably those who already have bright eyes, to avoid any asymmetry caused by the treatment. A subject must be 18 years of age or older and healthy in order to participate in the study. Women who are pregnant or nursing, individuals with a history of ocular disease or surgery, and patients who have known allergies to any of the components of the drug are excluded from participating in the study.

A fixed microdose of OCA2-siRNA eye drops is administered unilaterally to subjects in the study. The OCA2-siRNA drug is selected in accordance with the siRNA design (as further detailed below). Control is provided by the contralateral eye.

Volunteers are closely monitored for adverse effects or side effects associated with the treatment. Among the things that are monitored are their vision, eye pressure, and any changes that have occurred to their eye structure or function. The levels of siRNA in the body are also measured using body fluid samples such as blood, urine and tears. Additionally, the skin and hair are examined for signs of hypopigmentation. Further, by analysis that comprises monitoring body fluids and tissue samples for the presence of OCA2-siRNA, the biodistribution of the drug is assessed.

In order to assess any long-term effects of the treatment, volunteers are followed up after the treatment period. The trial is closely supervised by a team of researchers and physicians. Based on the data collected from the phase 0 trial, the siRNA eye drops are evaluated for safety and biodistribution. Taking this precaution ensures that the drug does not cause side effects or is not distributed to unwanted parts of the body before it is evaluated in subsequent studies with a larger sample size.

### Example 14

### Phase II/III Clinical Trial

The purpose of this study is to evaluate the effectiveness and safety of topically applied eye drops containing a full dose of OCA2-siRNA. The recruitment process involves the recruitment of several dozen to several hundred healthy human volunteers. Subjects must be 18 years of age or older and have non-blue eyes (e.g., brown, hazel) or heterochromia. Individuals who are pregnant or lactating, those who have had ocular disease or surgery or who have known allergies to any of the drug components are excluded from the study. A baseline slit lamp examination and clinical photographs are taken, and the images are then scanned into a computer and analyzed using calibrated software packages such as described previously (Andersen J D, et al. (2013) Forensic Sci Int Genet. 7 (5): 508-15) and documented. Throughout the course of the study, follow-up photographs are taken under the same conditions (i.e., light conditions, camera angle, time of day, etc.). This ensures that the data collected is accurate and consistent over the course of the study, and that any changes in the iris color can be attributed to the treatment rather than changes in the environment or the equipment used to collect the data.

The study is being conducted across multiple sites in a double-blind manner. Randomization is used to assign participants to treatment or placebo arms which receive a fixed dose of either OCA2-siRNA or placebo eye drops once per week for several months. The OCA2-siRNA drug is selected in accordance with (reference to siRNA design). In this case, the contralateral eye is used as a control. This allows the researchers to compare the effects of the OCA2-siRNA to the effects of the placebo and to determine if the OCA2-siRNA drug is having a positive effect.

In this study, the primary outcome is the achievement of brighter eye color as determined by a standardized colorimetry test/computerized test (such as measuring for the change in color-saturation of the iris over time as described earlier) or a visual examination by an ophthalmologist. Secondary endpoints include confluency of pigmentation and percentage changes in eye color, color-saturation, and brightness compared to the patient's baseline as measured by a standardized colorimetry test or computerized image analysis tests. Measurements are taken at baseline, throughout the study, and at the conclusion. Another secondary endpoint evaluates whether a significant change in color has occurred over a set period of time. The adverse effects of the drug are closely monitored throughout the clinical trial, both systemically and in the tissues of the eye. These include, but are not limited to, routine ophthalmological examinations (such as slit lamp examinations and optical coherence tomography) and monitoring for systemic side effects such as changes in vision, headaches, blood tests, or changes in the pigmentation of the skin and hair.

### Control Group

In order to assess the effectiveness and safety of the disclosed treatment, a control group is used. Control groups include for example subjects receiving no intervention, subjects receiving an empty carrier (i.e., transfection agent/nanocarrier alone, without nucleic acids), or subjects receiving a non-targeting siRNA sequence, containing the same nucleotide composition but with a different sequence (the letters are scrambled and the sequence is “BLASTed” to verify that the control did not target any of the mRNA of the mammal).

### Example 15

In Depth siRNA Sequences

The table of sequences is provided in Table 2 below.

TABLE 2
Examples of RNA sequences

| SEQ ID | target | | |
|---|---|---|---|
| NO: | position | target sequence | RNA guide |
| 1-2 | 378-400 | TCTAAAGATTCCTGCTTTACAGA | UGUAAAGCAGGAAUCUUUAGA |
| 3-4 | 387-409 | TCCTGCTTTACAGAAAACACTCC | AGUGUUUUCUGUAAAGCAGGA |
| 5-6 | 1296- | ATGATCTTAGTAGCCATATTTTC | AAAUAUGGCUACUAAGAUCAU |
| 7-8 | 1299- | ATCTTAGTAGCCATATTTTCAGA | UGAAAAUAUGGCUACUAAGAU |
| 9-10 | 1332- | TTCGATTATTGTGCTGTAAAGGC | CUUUACAGCACAAUAAUCGAA |
| 11-12 | 1522- | CAGAAGTGATCTTCACAAACATT | UGUUUGUGAAGAUCACUUCUG |
| 13-14 | 1571- | CCCTCCAAATGTCATTATTGTTT | ACAAUAAUGACAUUUGGAGGG |
| 15-16 | 1697- | ACTCCTTTACTGGAACAGAAAGC | UUUCUGUUCCAGUAAAGGAGU |
| 17-18 | 2002- | TGGGATTTGTTATCTTCATGTTT | ACAUGAAGAUAACAAAUCCCA |
| 19-20 | 2215- | TAGAATATGTTGGAGAACAAACT | UUUGUUCUCCAACAUAUUCUA |
| 21-22 | 2227- | GAGAACAAACTGCTTTGCTAATA | UUAGCAAAGCAGUUUGUUCUC |
| 23-24 | 2228- | AGAACAAACTGCTTTGCTAATAA | AUUAGCAAAGCAGUUUGUUCU |
| 25-26 | 2229- | GAACAAACTGCTTTGCTAATAAA | UAUUAGCAAAGCAGUUUGUUC |
| 27-28 | 2639- | CATCTATTGCTCGAAGACTAAAG | UUAGUCUUCGAGCAAUAGAUG |
| 29-30 | 2640- | ATCTATTGCTCGAAGACTAAAGG | UUUAGUCUUCGAGCAAUAGAU |
| 31-32 | 2652- | AAGACTAAAGGAAACTTCATCCA | GAUGAAGUUUCCUUUAGUCUU |
| 33-34 | 2671- | TCCATCACAACCCATTAGTCATA | UGACUAAUGGGUUGUGAUGGA |
| 35-36 | 2679- | AACCCATTAGTCATAAAACTACC | UAGUUUUAUGACUAAUGGGUU |
| 37-38 | 2680- | ACCCATTAGTCATAAAACTACCC | GUAGUUUUAUGACUAAUGGGU |
| 39-40 | 2707- | CCCACTGTTTGAAGAAGAAAAGG | UUUUCUUCUUCAAACAGUGGG |
| 41-42 | 2773- | TGACACTAACACTCTAATTCAAG | UGAAUUAGAGUGUUAGUGUCA |
| 43-44 | 2774- | GACACTAACACTCTAATTCAAGC | UUGAAUUAGAGUGUUAGUGUC |
| 45-46 | 2841- | AAGGACAAAATGTAGAAAGATGT | AUCUUUCUACAUUUUGUCCUU |
| 47-48 | 2850- | ATGTAGAAAGATGTGAGATAACT | UUAUCUCACAUCUUUCUACAU |
| 49-50 | 2853- | TAGAAAGATGTGAGATAACTTAC | AAGUUAUCUCACAUCUUUCUA |
| 51-52 | 2921- | TGCTATACATAGGAAAAGACACA | UGUCUUUUCCUAUGUAUAGCA |
| 53-54 | 2964- | TACTGTTTAACTGTCAATTCTCC | AGAAUUGACAGUUAAACAGUA |
| 55-56 | 3045- | TTCAAATTGTATGCTTTCTGAAT | UCAGAAAGCAUACAAUUUGAA |
| 57-58 | 3055- | ATGCTTTCTGAATATAGACTTTC | AAGUCUAUAUUCAGAAAGCAU |
| 59-60 | 3069- | TAGACTTTCTGGGAAAAGGTTTA | AACCUUUUCCCAGAAAGUCUA |
| 61-62 | 3092- | CTGCTCGTAAGGAAACATTTTAT | AAAAUGUUUCCUUACGAGCAG |
| 63-64 | 3093- | TGCTCGTAAGGAAACATTTTATG | UAAAAUGUUUCCUUACGAGCA |

The lengths of the sequences presented in the examples herein are illustrative and not limiting; actual sequences utilized may vary in length, based on the sequences described, and may include additional nucleotides or fewer nucleotides as described herein.

Additional siRNA sequences and targets thereof are disclosed by SEQ ID Nos. 78 to 200, while most of them were found in the following target region 375-420, 1270-1370, 1500-1600, 1900-2400 and 2610-3143. The present disclosure provides RNA therapeutic agents (siRNAs, ASOs, and other RNAs). In embodiments where the RNA therapeutic agent is a made of siRNA molecule it contains at least two nucleotide sequences, the sense sequence and the anti-sense sequence. A sense strand is selected from the group of sequences shown in Table 2. The antisense strand of the sense strand is selected from the sequences listed in Table 2. There is complementarity between these sequences in this aspect. An antisense sequence is highly complementary to the mRNA sequence generated by expression of OCA2.

Accordingly, a siRNA includes two oligonucleotides, where one oligonucleotide is described as the sense strand in Table 2, and the second oligonucleotide is described as the antisense strand of the sense strand in Table 2. There is an embodiment of the present invention in which substantially complementary sequences of the siRNA are contained on separate oligonucleotides. In another embodiment, there are two substantially complementary sequences of dsRNA on a single oligonucleotide.

In embodiments where the RNA therapeutic agent is made of ASO molecule it contains at least one nucleotide sequence. The ASO sequence is highly complementary to the mRNA sequence generated by expression of OCA2.

RNAs presented in Table 2 identify some of the site(s) within the OCA2 transcript that are generally prone to RNA-Induced Silencing Complex (RISC)-mediated cleavage or alternative pathways capable of reducing gene expression in a variety of ways which are well-known to those skilled in the field. Consequently, the present invention also includes RNA therapeutic agents that target one or more of these sites. In this context, an RNA therapeutic agent is defined as targeting a particular site within an RNA transcript. In view of the fact that the RNA therapeutic agent promotes cleavage at that particular site, it is likely that the transcript will be cleaved. Example of RNA therapeutic comprising at least 15 contiguous nucleotides from one of the sequences are listed in Table 2. As part of the process, these nucleotide sequences may be coupled with additional nucleotide sequences taken from the region contiguous to the selected sequence in the OCA2 gene. The length of each oligonucleotide compromising the RNA therapeutic agent may range from about 15 to about 30 nucleotides.

In embodiments where the RNA therapeutic agent is a siRNA the oligonucleotide is generally long enough to serve as a substrate for the Dicer enzyme and/or be used in the RISC complex. As an example, it is well known in the field that dsRNAs longer than 21-23 nucleotides may serve as substrates for Dicer. It is also apparent to a person with ordinary skill that the region of an RNA targeted for cleavage is usually part of a larger RNA molecule. Most often, this is a molecule of messenger RNA (mRNA).

As siRNA molecules are loaded onto the RNA-induced silencing complex (RISC), the antisense strand (the guide) of the siRNA molecules serves as a guide to recognize and bind to specific target RNAs, such as OCA2-mRNA. As soon as RISC binds to the target RNA, Argonaute, its catalytic component, cleaves the RNA, resulting in its degradation. This results in the knockdown of the corresponding gene, for example, OCA2. By degrading the target mRNA, RISC prevents it from being translated into proteins, thus preventing the expression of the gene. This, in turn, leads to the silencing of the gene, which has the desired effects in terms of gene regulation.

RNA therapeutic agents are capable of reducing the expression of target genes in a variety of ways that are perfectly within the knowledge of those skilled in the art. As a few examples, siRNAs may cleave the target mRNA strand by the RNA-induced silencing complex (RISC) or inhibit translation by blocking ribosome access to the mRNA. The ASO molecules may achieve similar results, for example, by forming a duplex with the target mRNA, which enables the RNase H enzyme to cleave it, or by binding to the mRNA and blocking its access to ribosomes, thus preventing translation.

NIH GenBank data is used in each of the examples reported herein, where OCA2-mRNA sequences for the appropriate mammal species are employed (e.g., rabbits, monkeys, humans, etc.). To design the sequence of the RNA therapeutic agent, the GenBank database is searched for the species of interest, and its OCA2 mRNA sequences are reviewed.

Alternatively, should the GenBank database not contain the requisite OCA2-mRNA sequences for the specific species under investigation, RNA sequencing (RNA-Seq) can be employed to elucidate the target mRNA sequence. This process involves isolating RNA from the species of interest, followed by its conversion into complementary DNA (cDNA) and subsequent high-throughput sequencing. Analyzing the sequence data enables the identification of the species-specific OCA2-mRNA sequence. Upon determination, small interfering RNAs (siRNAs) are meticulously designed, as described herein, to precisely target and silence the OCA2 gene in the specific species. Once the mammal OCA2-mRNA sequence has been identified, the RNA therapeutic agent intended to silence this gene is designed as follows:

### Types of RNA Nucleotides

The method involves administering ribonucleic acid (RNA) to the subject. For example, the RNA therapeutic agent is a siRNA comprising a sense strand and an antisense strand forming a double-stranded region consisting of at least 15 contiguous nucleotides that differ by no more than four nucleotides from the appropriate mRNA molecule nucleotide sequence, forming a region of complementarity. A prominent example of this is the human OCA2 mRNA variant 1 sequence in NCBI GenBank as denoted by SEQ ID NO: 65.

The RNA therapeutic agent mentioned inhibit the expression of the OCA2 gene within a cell, such as a cell within a mammal. This RNA therapeutic agent inhibits the expression of OCA2 in humans suffering from OCA2-associated disorders or in cases where reducing the expression of OCA2 may be beneficial, such as heterochromia, hyperpigmentation disorders of the eyes or for cosmetic purposes.

Thus, the invention entails RNA therapeutic agents, including antisense oligonucleotides, small interfering RNA (siRNA), microRNA (miRNA), short hairpin RNA (shRNA), Dicer-substrate RNA (dsRNA), piwi-interacting RNA (piRNA), long non-coding RNA (lncRNA), CRISPR-Cas9 guided RNA, and any other RNA therapeutic agents or technologies capable of inhibiting the expression of OCA2.

### Algorithm for Identifying Optimal Silencing Sequences

Identifying the optimal siRNA sequence for the knockdown of any gene expression is a standard practice well within the capabilities of those skilled in the art, involving the application of established guidelines, widely available software tools, and conventional wet-lab validation methods routinely used by those skilled in the art.

It is generally accepted that a target sequence consists of between 15 and 30 nucleotides in length. However, there is considerable variation in the suitability of particular sequences within this range for directing cleavage of any given target RNA. The guidelines presented herein and various software packages provide guidance on identifying optimal target sequences for any given gene target.

By making use of guidelines and algorithms that take into account factors like thermodynamic properties as well as GC content, and running a “BLAST” search to prevent off-target effects, it is possible to estimate, in silico, the most effective and specific sequences. However, the examples described herein do not limit the use of other optimizing rules and algorithms. Any other sequence of OCA2 RNA therapeutic agent that is capable of silencing the gene expression, or any other method of optimizing the RNA therapeutic agent ability to silence the OCA2-mRNA (such as different algorithms) that adheres to the principles of the subject technology would be suitable, as described herein. This means that although the examples presented are limited, any other method that follows the same principles can be used.

Rational for siRNA Sequence Selection:

Herein, the entire OCA2-mRNA nucleotide sequence is screened to identify sequences within it that adhere to any or all of the optimization techniques, algorithms and guidelines described herein, as well as other optimization techniques, algorithms and guidelines available to one skilled in the art. This process allows for the efficient identification of sequences that have been optimized for specific attributes such as stability, efficient target binding, increased likelihood of guide-strand uptake by the RISC, biodistribution (e.g., shorter siRNA molecules exhibit reduced molecular weight, potentially facilitating enhanced transport efficiency across ocular barriers.), effective gene silencing, minimal immunogenicity, increased target specificity, thereby reducing off-target effects and increasing safety, among other properties. These optimization guidelines and algorithms are designed to identify sequences with the most desirable characteristics for enhancing the efficiency and specificity of gene silencing.

RNA therapeutic agent strands should be 30 bp in length or shorter. This is because the interferon reaction caused by larger RNA molecules causes the expression of unspecific genes to alter. This reaction shifts the expression of many genes and may lead to side effects and ineffective silencing. The interferon reaction is triggered significantly more often when the double-stranded RNA therapeutic agent is longer than 30 bp because the longer molecule is quickly recognized by the cell as a foreign agent. The cell then activates its interferon response, which causes the expression of many genes to shift in an effort to fight off the “foreign” molecule. This can lead to unspecific gene silencing and side effects. With shorter RNA therapeutic agent, this is much less likely to occur (Persengiev S P, et al. (2004) RNA N Y N. 12-8).

Due to the fact that in some locations there are regulatory proteins binding or the secondary structure is extremely complex, siRNA sequences complementary to several different locations along the 3.1K nucleotides of human OCA2-mRNA should be used, as this could reduce the possibility of capture.

As a rule, GC bases should constitute 30-65% of the composition, preferably 30-55%. This is due to the fact that strands with a high GC are more tightly bonded, as G nucleotides on one strand establish three covalent bonds with C nucleotides on the other strand. This is in contrast to the two covalent bonds between A and T nucleotides. Thus, high GC content inhibits the unwinding of duplexes, resulting in a reduced level of gene expression inhibition.

The use of sequences consisting of more than four contiguous A or T nucleotides should be avoided. This is because these sequences indicate the end of the signal for the RNA polymerase III enzyme.

Sequences containing GTCCTTCAA, TGTGT, and CTGAATT among others, are known to induce immune responses and are therefore best avoided (Birmingham, A., et al. Nat Protoc 2, 2068-2078 (2007).

If a candidate duplex contains five or more consecutive bases of the same base, it should be eliminated. The elimination of candidate duplexes containing seeds may reduce off-target effects (Vaish N, et al. (2011) Nucleic Acids Res. 39 (5): 1823-32).

This is because the homogeneity of the duplex will weaken its stability, making it less likely to bind to its target. Additionally the seed can increase the frequency of mismatches, which can also weaken the stability of the duplex.

### BLAST

In order to ensure that the target sequence is not present in any other gene mRNA and to ensure that the silencing is specific to the OCA2-mRNA, the target sequence is entered into NCBI BLAST search engine and sequences with homology to other genes are excluded. A description of the BLAST algorithm can be found in Altschul et al. (Altschul et al. (1997), Nucleic Acids Res. 25:3389-3402).

To increase specificity, the candidate siRNA sequences are blasted against an extensive and unique mRNA database to remove non-unique sequences. A candidate siRNA that shares more than 11-15 contiguous nucleotides with a different mRNA should be disregarded, as recommended by Jackson et al. (Jackson A L, et al. (2003) Nat Biotechnol 21 (6): 635-7).

As the “BLAST” database has grown, it has significantly reduced the number of candidate siRNAs that exclusively target one specific mRNA. As a result, only siRNAs with homologous sequences of more than 2-5 unigene sequences are preferably discarded.

Enhancing siRNA Design Through Advanced in Silico Simulations

By simulating the secondary structure of the siRNA sequence and measuring the free energy in the molecule, better candidates can be selected, as molecules with higher free energy tend not to fold as easily and, therefore, are better candidates. Furthermore, it is possible to select siRNA candidates with greater activity by analyzing the thermodynamic properties of the interactions between two RNA sequences, i.e., the siRNA and its complementary sequence on the mRNA molecule. Thermodynamic properties also determine which of the strands will be loaded into the RISC with greater likelihood, further influencing the efficacy of gene silencing.

Utilizing ocular pharmacokinetics models, its possible to evaluate the biodistribution profile of various siRNA sequences and particularly lengths against ocular barriers and iris biodistribution. This approach identifies siRNAs with the optimal potential for effective delivery and gene silencing, streamlining the selection of candidates for further development.

Thermodynamics plays a crucial role in selecting siRNA sequences due to its impact on the stability of the siRNA-target mRNA duplex. The balance between enthalpic (bonding) and entropic (disordering) forces determines hybridization, which in turn influences the specificity and effectiveness of siRNA-mediated gene silencing. Statistical analyses of published siRNA sequences reveal that functional duplexes typically exhibit lower internal stability at their 5′ antisense ends, compared to non-functional ones. This lower stability is pivotal for target mRNA recognition, as it allows for the necessary flexibility in the 5′ end of the sense strand, facilitating the formation of a functional duplex.

Another effective way to identify and differentiate siRNA candidates with high efficiency from less efficient candidates is by simulating their docking to the argonaute protein. Using this computational approach, insight can be gained into the physical interaction between the argonaute protein and the siRNA candidate. As a result, it is possible to evaluate the strength and stability of the complex formed between the two molecules. Moreover, it allows for the identification of potential weaknesses in the interaction. This allows for the optimization of the siRNA candidate argonaute protein binding capacity, thereby choosing candidates with enhanced silencing ability. Modeling the docking can also provide information regarding the orientation and spatial arrangement of the siRNA candidate whilst bound to the argonaute protein. Therefore, it is beneficial to identify which siRNA sequences bind to the argonaute protein in a conformation that is most efficient. This will provide an additional opportunity to predict whether the target gene will be optimally silenced.

### Empirical Approach

Alternatively, an empirical approach is also conducted, in which a “window” or “mask” of a particular size (for example, 21 nucleotides) is placed literally or figuratively (including, for example, in silico) on the target RNA sequence in order to identify sequences that can be used as target sequences within this size range. For any given target size, the sequence “window” can be moved gradually one nucleotide upstream or downstream to identify the next potential target sequence, until the entire set of possible sequences is identified.

Utilizing the “window method,” and then testing the siRNA candidates using the assays described in the examples herein or other methods well known in the art, sequences of RNA therapeutic agents can be identified that are most effective at silencing the expression of OCA2. The sequences identified, for example, in Table 2 are predicted, based on the above-mentioned algorithmic approach and optimization methods, to be effective target sequences. However, further optimization of gene inhibition efficiency is contemplated by incrementally “walking the window” one nucleotide upstream or downstream of the given sequences in order to identify sequences with similar or better inhibition characteristics.

Further, it is envisaged that for any sequence identified, such as in Table 2, further optimization can be achieved by adding or removing nucleotides systematically to generate longer or shorter sequences, and testing those sequences by walking a window of the longer or shorter size up or down the target RNA from the identified point. Further improvements in the efficiency of inhibition can be achieved by coupling this approach to generating new candidate targets with testing for effectiveness of RNA therapeutic agents based on those target sequences in an inhibition assay, as is known in the art and/or described in the present invention.

### Modifications

By introducing modified nucleotides as described herein or as known in the art, the sequences described herein can be further adjusted. Adjustments may include adding or modifying overhangs, introducing modified nucleotides or making other modifications known in the art or discussed herein in order to further optimize siRNA silencing effects.

These adjustments aim to enhance the drug stability within tissues or intracellular environments by reducing its degradation rate, particularly in the endosomal setting, and to extend its half-life in the aqueous humor and in tissues such as the iris, sclera, conjunctiva, and cornea. Additionally, they seek to improve drug delivery into cells, target specific cell types (e.g., iris stromal melanocytes) or locations, boost interaction with enzymes involved in the silencing pathway, increase the likelihood of guide strand uptake by the RISC, and decrease off-target effects. These modifications also aim to reduce immunogenicity, minimize systemic absorption, and enhance endosomal escape for better efficacy.

Nucleotide Choosing, Length, Fitting to mRNA, Overhang

In embodiments where the RNA therapeutic agent is dsRNA, the dsRNA contains an antisense strand whose sequence complements at least a portion of the mRNA produced by the expression of an OCA2 gene. In embodiments where the RNA therapeutic agent is an antisense oligonucleotide, the oligonucleotide sequence complements at least a portion of the mRNA generated by the expression of an OCA2 gene.

In general, the complementarity region is about 30 nucleotides in length or less (e.g., about 30, 29, 28, 27, 26, 25, 24, 23, 22, 21, 20, 19, 18, 17, 16 or 15 nucleotides). A cell that expresses the OCA2 gene (such as a human cell) is inhibited by at least about 10% when in the presence of an RNA therapeutic agent, as assessed by, for example, PCR or branched DNA (bDNA)-based procedures for RNA quantification. Protein expression resulting from OCA2 gene activity can be evaluated by Western blotting and enzyme-linked immunosorbent assay (ELISA) for quantification, and immunohistochemistry (IHC) for localization within tissues. Flow cytometry can be used to analyze cell surface markers and intracellular proteins, assessing the impact of the RNA therapeutic agent on the protein level associated with OCA2 gene expression.

Under the conditions under which the dsRNA is used, two complementary RNA strands will form a duplex structure. The antisense strand of a dsRNA includes a region of complementarity that is substantially complementary, and generally fully complementary to the target sequence. The target sequence can be derived from the sequence of an mRNA formed during the expression of an OCA2 gene. As the sense strand is complementary to the antisense strand, the two strands combine and form a duplex structure when combined under appropriate conditions. The complementary sequences of a dsRNA are also possible to contain on a single nucleic acid molecule rather than on separate oligonucleotides, as known in the art.

The region of complementarity to the target sequence is between 15 and 30 nucleotides in length. The invention is intended to encompass portions of ranges and lengths that are intermediate to those recited.

It is also possible for a dsRNA (or ASO) to contain one or more single-stranded nucleotide overhangs, such as 1, 2, 3, or 4 nucleotides. There have been reports that dsRNAs having at least one nucleotide overhang have unexpectedly superior inhibitory properties compared to their blunt-ended counterparts. Nucleotide overhangs may consist of nucleotide/nucleoside analogs, including deoxynucleotide/nucleoside analogs. There can be overhangs on either the sense or antisense strand or a combination of both. A dsRNA overhang can contain nucleotides at the 5′, 3′, or both ends of either the antisense or sense strands. Depending on the embodiment, longer, extended overhangs may be possible.

In RNA therapeutic development, selecting between blunt-ended dsRNAs and those with nucleotide overhangs is pivotal. While blunt-ended dsRNAs are simpler and may elicit fewer immunogenic responses, making them preferred for minimizing immune reactions, dsRNAs with overhangs surpass them by offering enhanced gene silencing efficacy. This superiority stems from better RISC complex engagement and a closer resemblance to natural RNA processing products, advantages that facilitate more effective gene targeting. However, this enhanced functionality comes with its drawbacks; overhangs are associated with increased immunogenic responses and a higher potential for off-target interactions. Thus, the decision between the two types embodies a careful trade-off, aiming to maximize therapeutic efficacy while managing the risks of immune activation and unintended gene interactions.

### Ordering

The OCA2 siRNA is designed to target the OCA2 gene transcript from the OCA2 gene (NCBI GenBank accession number, NM_000275.3). Its nucleotide sequence is as follows: UAAAGAUUCCUGCUUUACAGA (sense strand, as denoted by SEQ ID 67) and UGUAAAGCAGGAAUCUUUAGA (antisense strand, as denoted by SEQ ID 2). Negative control RNA therapeutic agent is purchased from Thermo-Fisher Scientific.

### Purification

Purification of the siRNA (or ASO) can be achieved through several effective methods. Binding to glass fibers followed by elution is a common approach, as is gel purification using 15-20% acrylamide gel, which helps remove excess nucleotides, short oligomers, proteins, and salts. Gel electrophoresis is particularly useful for siRNAs synthesized chemically, offering precise size selection. Additionally, ion-exchange chromatography and reverse-phase HPLC (High-Performance Liquid Chromatography) have become standard for purifying larger batches, providing high purity and specificity by separating molecules based on charge or hydrophobicity, respectively. Another method, size-exclusion chromatography, is employed for its ability to separate based on molecular size, useful in removing smaller impurities. The selection of a purification method may depend on the specific requirements for purity, yield, and downstream application, and any other method known to one skilled in the art may also be employed for the purpose of RNA purification.

Pooling siRNA Sequences

In some usages of the invention, such as during in vitro experiments and in vivo studies, pooling 2 to 10, preferably 2 to 4, siRNA sequences targeting the OCA2 gene is advised to enhance silencing efficacy by addressing multiple mRNA regions, thereby increasing knockdown efficiency.

1-56. (canceled)

57. A method of modifying the eye color of a subject comprising administering to said subject an effective amount of at least one Oculocutaneous Albinism 2 (OCA2) protein modulating agent, or any vehicle, matrix, nano- or micro-particle thereof or any composition comprising the same.

58. The method of claim 57, wherein said at least one modulating agent comprises at least one nucleic acid molecule, optionally wherein at least one of:
(i) said nucleic acid molecule comprises at least one of a single stranded RNA (ssRNA), a single stranded DNA (ssDNA), a double stranded DNA (dsDNA), a double stranded RNA (dsRNA), nucleic acid molecule having at least one modified nucleotide/s and any combinations thereof; and/or
(ii) said nucleic acid molecule comprises at least one of a small interfering RNA (siRNA), an antisense oligonucleotide (ASO), a guide RNA, a short hairpin RNA (shRNA), microRNA (miRNA), Peptide-Nucleic Acid (PNA) and locked nucleic acid (LNA), optionally wherein said nucleic acid molecule comprises at least one siRNA molecule, wherein said siRNA molecule targets at least on sequence coding or controlling directly or indirectly the levels and/or activity and/or stability of OCA2.

59. The method of claim 57, wherein at least one of:
(i) the at least one modulating agent targets at least one region in the OCA2 transcript;
(ii) said modulating agent inhibits and/or reduces the expression and/or activity and/or stability of OCA2, optionally wherein said inhibition reduces the levels of melanin in the iris tissue of said subject;
(iii) said OCA2 modulating agent reduces the expression and/or activity and/or stability of OCA2, said reduction results in lightening the eye color of said subject;
(iv) said at least one OCA2 modulating agent targets the iris stroma of said subject;
(v) said method further comprising the step of exposing said subject to a light source;
(vi) said at least one OCA2 modulating agent is administered via ocular administration; and/or
(vii) said at least one OCA2 modulating agent is administered via topical administration, optionally wherein said at least one OCA2 modulating agent is comprised in an eye drop formulation.

60. The method of claim 57, for cosmetic purposes.

61. The method of claim 57, wherein the subject is healthy.

62. The method of claim 57, wherein the subject suffers from a disease, optionally wherein said disease is any one of hyperpigmentation ocular disorders, ocular glaucoma, Horner syndrome or heterochromia.

63. A composition for modifying the eye color of a subject comprising at least one Oculocutaneous Albinism 2 (OCA2) protein modulating agent.

64. The composition of claim 63, wherein said at least one modulating agent comprises at least one nucleic acid molecule, optionally wherein at least one of:
(i) said nucleic acid molecule comprises at least one of a single stranded DNA (ssDNA), a single stranded RNA (ssRNA), a double stranded DNA (dsDNA), a double stranded RNA (dsRNA), nucleic acid molecule having at least one modified nucleotide/s and any combinations thereof; and/or
(ii) said nucleic acid molecule comprises at least one of a small interfering RNA (siRNA), an antisense oligonucleotide (ASO), a guide RNA, a short hairpin RNA (shRNA), microRNA (miRNA), Peptide-Nucleic Acid (PNA) and locked nucleic acid (LNA), optionally wherein said nucleic acid molecule comprises at least one siRNA molecule, wherein said siRNA molecule targets at least one sequence coding or controlling directly or indirectly the levels and/or activity and/or stability of OCA2.

65. The composition of claim 63, wherein at least one of:
(i) the at least one OCA2 modulating agent targets at least one region in the OCA2 transcript;
(ii) said OCA2 modulating agent inhibits and/or reduces the expression and/or activity and/or stability of OCA2;
(iii) said inhibition reduces the levels of melanin in the iris tissue of said subject;
(iv) said OCA2 modulating agent reduces the expression and/or activity and/or stability of OCA2, said reduction results in lightening the eye color of said subject;
(v) said at least one OCA2 modulating agent targets the iris stroma of said subject;
(vi) said at least one OCA2 modulating agent is encapsulated within or coated with at least one nano-carrier;
(vii) said at least one OCA2 modulating agent is administered via ocular administration;
(viii) said at least one OCA2 modulating agent is administered via topical administration, optionally wherein said at least one OCA2 modulating agent is comprised in an eye drop formulation.

66. The composition of claim 63, for use as a cosmetic.

67. A pharmaceutical composition comprising at least one Oculocutaneous Albinism 2 (OCA2) protein modulating agent and at least one of pharmaceutically acceptable carrier/s, diluent/s and/or excipient/s.

68. The pharmaceutical composition of claim 67, wherein said at least one modulating agent comprises at least one nucleic acid molecule, optionally wherein at least one of:
(i) said nucleic acid molecule comprises at least one of a single stranded DNA (ssDNA), a single stranded RNA (ssRNA), a double stranded DNA (dsDNA), a double stranded RNA (dsRNA), nucleic acid molecule having at least one modified nucleotide/s and any combinations thereof; and/or
(ii) said nucleic acid molecule comprises at least one of a small interfering RNA (siRNA), an antisense oligonucleotide (ASO), a guide RNA, a short hairpin RNA (shRNA), microRNA (miRNA), Peptide-Nucleic Acid (PNA) and locked nucleic acid (LNA), optionally wherein said nucleic acid molecule comprises at least one siRNA molecule, wherein said siRNA molecule targets at least one sequence coding or controlling directly or indirectly the levels and/or activity and/or stability of OCA2.

69. A nucleic acid molecule for modifying the eye color of a subject comprising at least one Oculocutaneous Albinism 2 (OCA2) protein modulating agent.

70. The nucleic acid molecule of claim 69, comprising at least one of a single stranded DNA (ssDNA), a single stranded RNA (ssRNA), a double stranded DNA (dsDNA), a double stranded RNA (dsRNA), nucleic acid molecule having at least one modified nucleotide/s and any combinations thereof, optionally wherein at least one of:
(i) said nucleic acid molecule comprises at least one of a small interfering RNA (siRNA), an antisense oligonucleotide (ASO), a guide RNA, a short hairpin RNA (shRNA), microRNA (miRNA), Peptide-Nucleic Acid (PNA) and locked nucleic acid (LNA); and/or
(ii) said nucleic acid molecule comprises at least one siRNA molecule, wherein said siRNA molecule targets at least on sequence coding or controlling directly or indirectly the levels and/or activity and/or stability of OCA2.

71. The nucleic acid molecule of claim 69, wherein at least one of:
(i) said nucleic acid molecule targeting at least one region in the OCA2 transcript;
(ii) said nucleic acid molecule inhibiting and/or reducing the expression and/or activity and/or stability of OCA2, optionally wherein said inhibition reduces the levels of melanin in the iris tissue of said subject;
(iii) said at least one OCA2 modulating agent reduces the expression and/or activity and/or stability of OCA2, said reduction results in lightening the eye color of said subject; and/or
(iv) said nucleic acid molecule is encapsulated within or coated with at least one nano-carrier;

72. The nucleic acid molecule of claim 69, for use as a cosmetic.

73. A method of treating a disease/disorder in a subject in need thereof comprising administering to said subject an effective amount of at least one Oculocutaneous Albinism 2 (OCA2) protein modulating agent or any vehicle, matrix, nano- or micro-particle thereof or any composition comprising the same.

74. The method of claim 73, wherein said disease/disorder is an eye disorder, optionally wherein said eye disorder is any one of heterochromia, Horner syndrome or hyperpigmentation.

75. The method of claim 73, wherein said at least one modulating agent comprises at least one nucleic acid molecule, optionally wherein at least one of:
(i) said nucleic acid molecule comprises at least one of a single stranded DNA (ssDNA), a single stranded RNA (ssRNA), a double stranded DNA (dsDNA), a double stranded RNA (dsRNA), nucleic acid molecule having at least one modified nucleotide/s and any combinations thereof; and/or
(ii) said nucleic acid molecule comprises at least one of a small interfering RNA (siRNA), an antisense oligonucleotide (ASO), a guide RNA, a short hairpin RNA (shRNA), microRNA (miRNA), Peptide-Nucleic Acid (PNA) and locked nucleic acid (LNA), optionally wherein said nucleic acid molecule comprises at least one siRNA molecule, wherein said siRNA molecule targets at least one sequence coding or controlling directly or indirectly the levels and/or activity and/or stability of OCA2.

76. The method of claim 73, wherein at least one of:
(i) said at least one OCA2 modulating agent comprises at least one of nucleic acid molecule or any vector or host cell comprising the same, optionally wherein said at least one nucleic acid molecule comprises at least one of a single stranded DNA (ssDNA), a single stranded RNA (ssRNA), a double stranded DNA (dsDNA), a double stranded RNA (dsRNA), nucleic acid molecule having at least one modified nucleotide/s and any combinations thereof, optionally wherein at least one of:
(a) said nucleic acid molecule comprises at least one of a small interfering RNA (siRNA), an antisense oligonucleotide (ASO), a guide RNA, a short hairpin RNA (shRNA), microRNA (miRNA), Peptide-Nucleic Acid (PNA) and locked nucleic acid (LNA); and/or
(b) said nucleic acid molecule comprises at least one siRNA molecule, wherein said siRNA molecule targets at least on sequence coding or controlling directly or indirectly the levels and/or activity and/or stability of OCA2,
or any vector or host cell comprising the same;
(ii) said at least one OCA2 modulating agent is administered via ocular administration;
(iii) said at least one OCA2 modulating agent is administered via topical administration; and/or
(iv) said at least one OCA2 modulating agent is comprised in an eye drop formulation.
